SEQUENCE LISTING (79 sequences)

SEQ ID NO: 1 - protein, 823 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGYNTTDKEMEVLHLRNVSFEDAGEYTCLAGNSIGLSHHSAWLTVLEALEERPAVIVITSPLYLEIIIYCTGAFLISCMVGSVIVYKMKSGTKKSDFHSQMAVEIKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKEIKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 2 - protein, 1004 aa, Homo sapiens
FSGDGRAIWSKNPNFTPVNESQLFLYDTFPKNFFWGIGTGALQVEGSWKKDGKGPSIWDEIFIHTEILKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIEINPYLVAWHGYGTGMHAPGEKGNLAAVYTVGHNLIKAHSKVWEINYNTEIFRPHQKGWLSITLGSHWIEPNRSENTMDIFKCQQSMVSVLGWFANPIHGDGDYPEGMRKKLFSVLPIFSEAEKEIEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPEILYVWNATGNRLLEIRVEGVRLKTRPAQCTDFVNIKKQLEMLARMKVTHYRFALDWASVLPTGNLSAVNRQALRYYRCVVSEGLKLGISAMVTLYYPTHAEILGLPEPLLHADGWLNPSTAEAFQAYAGLCFQELGDLVKLWITINEPNRLSDIYNRSGNDTYGAAEINLLVAHALAWRLYDRQFRPSQRGAVSLSLHADWAEPANPYADSHWRAAERFLQFEIAWFAEPLFKTGDYPAAMREYIASKEIRRGLSSSALPRLTEAERRLLKGTVDFCALNEIFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPLIFLGCCFFSTLVLLLSIAIFQRQKRRKFWKAKNLQHIPLKKGKRVVS

SEQ ID NO: 3 - protein, 115 aa, Artificial Sequence
  Description of Artificial Sequence Synthetic polypeptide
EVQLVQSGAEVKKPGASVKVSCKVSGYTFTDYYMHWVQQAPGKGLEWMGLVDPEDGETIYAEKFQGRVTITADTSTDTAYMELSSLRSEDTAVYYCARDDYMDVWGKGTLVTVSS

SEQ ID NO: 4 - protein, 108 aa, Artificial Sequence
  Description of Artificial Sequence Synthetic polypeptide
ETTLTQSPDTLSLSPGEGATLSCRASQSVSGSALAWYQQKPGQAPRLLIYDASSRATGVPDRFSGSGSGADFSLTISRLEPEDFAVYSCQQYGSSPLTFGPGTKVDVK

SEQ ID NO: 5 - protein, 110 aa, Artificial Sequence
  Description of Artificial Sequence Synthetic polypeptide
QVQLVQSGAEVKKPGSSVKVSCKASGQTFTGYYMHWVRQAPGQGLEWMGRIPILGIANYAQKFQGRVTITADKSTSTAYMELSSLRSEDTAVYYCARGGDLGGMDVWGQG

SEQ ID NO: 6 - protein, 111 aa, Artificial Sequence
  Description of Artificial Sequence Synthetic polypeptide
EIVLTQSPLSLPVTPGEPASISCRSSQSLRHSNGYNYLDWYLQKPGQSPQLLIYLASNRASGVPDRFSGSGSGTDFTLKISRVEAEDVGVYYCMQALQIPPTFGPGTKVDK

SEQ ID NO: 7 - protein, 107 aa, Artificial Sequence
  Description of Artificial Sequence Synthetic polypeptide
EVQLVESGGGLVQPGGSLRLSCAASGFTFTSTWISWVPGKGLEWVGEIDPYDGDTYYADSVKGRFTISADTSKNLQMNSLRAEDTAVYYCASSGYGGSDYAMDYWGQ

SEQ ID NO: 8 - protein, 107 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKWEIK

SEQ ID NO: 9 - protein, 109 aa, Artificial Sequence
  Description of Artificial Sequence Synthetic polypeptide
EVQLVESGGGLVQPGGSLRLSCAASGFTFSNNYIHWVPGKGLEWVADIYPNDGDTDYADSVKGRFTISADTSKNLQMNSLRAEDTAVYYCAREHFDAWVHYYVMDYWGQ

SEQ ID NO: 10 - protein, 102 aa, Artificial Sequence
  Description of Artificial Sequence Synthetic polypeptide
EVQLVESGGGLVQPGGSLRLSCAASGFTFTSNWISWVPGKGLEWVAEIDPYDGATDYADSVKGRFTISADTSKNLQMNSLRAEDTAVYYCATGTDWMDYWGQ

SEQ ID NO: 11 - protein, 124 aa, Artificial Sequence
  Description of Artificial Sequence Synthetic polypeptide
QVQLVESGGGVVQPGRSLRLSCAASGFTFSNYGIHWVRQAPGKGLEWVAVIWYDGSIKYYADSVKGRFTISRDNSKNTLYLQMNSLRAEDTAVYYCARDRAAAGLHYYYGMDVWGQGTTVTVSS

SEQ ID NO: 12 - protein, 108 aa, Artificial Sequence
  Description of Artificial Sequence Synthetic polypeptide
EIVLTQSPGTLSLSPGERATLSCRASQSVSSTYLAWYQQKPGQAPRLLIYGASSRATGIPDRFSGSGSGTDFTLTISRLEPEDFAVYYCQQSGSSPLTFGGGTEVEIK

SEQ ID NO: 13 - protein, 120 aa, Artificial Sequence
  Description of Artificial Sequence Synthetic polypeptide
QVQLQQSGAEVKKPGASVKVSCKASGYTFTSYDINWVRQAPGQGLEWIGWIYPGDGSTKYNEKFKGKATITRDTSASTAYMELSSLRSEDTAVYFCARSDYYGSRSFAYWGQGTLVTVSS

SEQ ID NO: 14 - protein, 111 aa, Artificial Sequence
  Description of Artificial Sequence Synthetic polypeptide
DIVMTQSPDSLAVSLGERATINCRASKSVSTSGYVYMHWYQQKPGQPPKLLIYLASYLESGVPDRFSGSGSGTDFTLTISSVQAEDVAVYYCQHSRDLTFPFGGGTKLEIK

SEQ ID NO: 15 - protein, 8 aa, Artificial Sequence
  Description of Artificial Sequence Synthetic peptide
  SITE (1)..(7): This region may encompass 1-7 residues
GGGGGGGS

SEQ ID NO: 16 - protein, 8 aa, Artificial Sequence
  Description of Artificial Sequence Synthetic peptide
  SITE (2)..(8): This region may encompass 1-7 residues
SGGGGGGG

SEQ ID NO: 17 - protein, 5 aa, Artificial Sequence
  Description of Artificial Sequence Synthetic peptide
GGGGS

SEQ ID NO: 18 - protein, 4 aa, Artificial Sequence
  Description of Artificial Sequence Synthetic peptide
GGGG

SEQ ID NO: 19 - protein, 5 aa, Artificial Sequence
  Description of Artificial Sequence Synthetic peptide
GGGGG

SEQ ID NO: 20 - protein, 6 aa, Artificial Sequence
  Description of Artificial Sequence Synthetic peptide
GGGGGG

SEQ ID NO: 21 - protein, 7 aa, Artificial Sequence
  Description of Artificial Sequence Synthetic peptide
GGGGGGG

SEQ ID NO: 22 - protein, 8 aa, Artificial Sequence
  Description of Artificial Sequence Synthetic peptide
GGGGGGGG

SEQ ID NO: 23 - protein, 9 aa, Artificial Sequence
  Description of Artificial Sequence Synthetic peptide
GGGGGGGGG

SEQ ID NO: 24 - protein, 15 aa, Artificial Sequence
  Description of Artificial Sequence Synthetic peptide
GGGGSGGGGSGGGGS

SEQ ID NO: 25 - protein, 11 aa, Artificial Sequence
  Description of Artificial Sequence Synthetic peptide
  SITE (1)..(10): This region may encompass 1-10 residues
GGGGGGGGGGS

SEQ ID NO: 26 - protein, 11 aa, Artificial Sequence
  Description of Artificial Sequence Synthetic peptide
  SITE (2)..(11): This region may encompass 1-10 residues
SGGGGGGGGGG

SEQ ID NO: 27 - protein, 4 aa, Artificial Sequence
  Description of Artificial Sequence Synthetic peptide
CPPC

SEQ ID NO: 28 - protein, 4 aa, Artificial Sequence
  Description of Artificial Sequence Synthetic peptide
CPSC

SEQ ID NO: 29 - protein, 20 aa, Artificial Sequence
  Description of Artificial Sequence Synthetic peptide
EPKSCDKTHTCPPCPAPPVA

SEQ ID NO: 30 - protein, 17 aa, Artificial Sequence
  Description of Artificial Sequence Synthetic peptide
ESKYGPPCPPCPAPPVA

SEQ ID NO: 31 - protein, 15 aa, Artificial Sequence
  Description of Artificial Sequence Synthetic peptide
CPPCPAPGGGGPSVF

SEQ ID NO: 32 - protein, 14 aa, Artificial Sequence
  Description of Artificial Sequence Synthetic peptide
CPPCPAPGGGPSVF

SEQ ID NO: 33 - protein, 13 aa, Artificial Sequence
  Description of Artificial Sequence Synthetic peptide
CPPCPAPGGPSVF

SEQ ID NO: 34 - protein, 12 aa, Artificial Sequence
  Description of Artificial Sequence Synthetic peptide
CPPCPAPGPSVF

SEQ ID NO: 35 - protein, 232 aa, Artificial Sequence
  Description of Artificial Sequence Synthetic polypeptide
DKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 235 aa, Artificial Sequence
  Description of Artificial Sequence Synthetic polypeptide
DKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 329 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 40 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 41 - protein, 5 aa, Artificial Sequence
  Description of Artificial Sequence Synthetic 5xHis tag
HHHHH

SEQ ID NO: 42 - protein, 6 aa, Artificial Sequence
  Description of Artificial Sequence Synthetic 6xHis tag
HHHHHH

SEQ ID NO: 43 - protein, 45 aa, Artificial Sequence
  Description of Artificial Sequence Synthetic polypeptide
  SITE (1)..(45): This sequence may encompass 3-9 “Gly Gly Gly Gly Ser” repeating units
GGGGSGGGGSGGGGSGGGGSGGGGSGGGGSGGGGSGGGGSGGGGS

SEQ ID NO: 44 - protein, 20 aa, Artificial Sequence
  Description of Artificial Sequence Synthetic peptide
GGGGSGGGGSGGGGSGGGGS

SEQ ID NO: 45 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 48 - protein, 235 aa, Artificial Sequence; identical to SEQ ID NO: 36
  Description of Artificial Sequence Synthetic polypeptide

SEQ ID NO: 49 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 228 aa, Artificial Sequence
  Description of Artificial Sequence Synthetic polypeptide
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 51 - protein, 228 aa, Artificial Sequence
  Description of Artificial Sequence Synthetic polypeptide
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 52 - protein, 229 aa, Artificial Sequence
  Description of Artificial Sequence Synthetic polypeptide
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 229 aa, Artificial Sequence
  Description of Artificial Sequence Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 228 aa, Artificial Sequence
  Description of Artificial Sequence Synthetic polypeptide
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 7 aa, Artificial Sequence
  Description of Artificial Sequence Synthetic peptide
GGGGSGG

SEQ ID NO: 56 - protein, 15 aa, Artificial Sequence
  Description of Artificial Sequence Synthetic peptide
GGGGSGGGGSGGGGS

SEQ ID NO: 57 - protein, 30 aa, Artificial Sequence
  Description of Artificial Sequence Synthetic polypeptide
GGGGSGGGGSGGGGSGGGGSGGGGSGGGGS

SEQ ID NO: 58 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 60 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 62 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 63 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 64 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 65 - protein, 231 aa, Artificial Sequence
  Description of Artificial Sequence Synthetic polypeptide
EPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 66 - protein, 22 aa, Artificial Sequence
  Description of Artificial Sequence Synthetic peptide
EPKSCDKTHTCPPCPAPPVAGP

SEQ ID NO: 67 - protein, 22 aa, Artificial Sequence
  Description of Artificial Sequence Synthetic peptide
EPKSCDKTHTCPPCPAPPVAGP

SEQ ID NO: 68 - protein, 23 aa, Artificial Sequence
  Description of Artificial Sequence Synthetic peptide
EPKSCDKTHTCPPCPAPELLGGP

SEQ ID NO: 69 - protein, 19 aa, Artificial Sequence
  Description of Artificial Sequence Synthetic peptide
ERKCCVECPPCPAPPVAGP

SEQ ID NO: 70 - protein, 20 aa, Artificial Sequence
  Description of Artificial Sequence Synthetic peptide
ESKYGPPCPPCPAPEFLGGP

SEQ ID NO: 71 - protein, 19 aa, Artificial Sequence
  Description of Artificial Sequence Synthetic peptide
ESKYGPPCPPCPAPPVAGP

SEQ ID NO: 72 - protein, 20 aa, Artificial Sequence
  Description of Artificial Sequence Synthetic peptide
ESKYGPPCPSCPAPEFLGGP

SEQ ID NO: 73 - protein, 22 aa, Artificial Sequence
  Description of Artificial Sequence Synthetic peptide
GGGGSGGGGSGGGGSGGGGSGG

SEQ ID NO: 74 - protein, 37 aa, Artificial Sequence
  Description of Artificial Sequence Synthetic polypeptide
GGGGSGGGGSGGGGSGGGGSGGGGSGGGGSGGGGSGG

SEQ ID NO: 75 - protein, 45 aa, Artificial Sequence; identical to SEQ ID NO: 43
  Description of Artificial Sequence Synthetic polypeptide

SEQ ID NO: 76 - protein, 330 aa, Artificial Sequence [sequence omitted]

SEQ ID NO: 77 - protein, 326 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 78 - protein, 327 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 4 aa, Artificial Sequence
  Description of Artificial Sequence Synthetic peptide
ELLG

(19) United States
(12) Patent Application Publication
(10) Pub. No.: US 2022/0372168 A1
(43) Pub. Date: Nov. 24, 2022

(54) MULTISPECIFIC FGF21 RECEPTOR AGONISTS AND THEIR USES

(71) Applicant: Regeneron Pharmaceuticals, Inc., Tarrytown, NY (US)

(72) Inventors: Yang SHEN, Scarsdale, NY (US); Ann-Hwee LEE, Ardsley, NY (US); Chia-Yang LIN, Scarsdale, NY (US); Naga Suhasini AVVARU, Briarcliff Manor, NY (US); Samuel DAVIS, New York, NY (US)

(73) Assignee: Regeneron Pharmaceuticals, Inc., Tarrytown, NY (US)

(21) Appl. No.: 17/735,510

(22) Filed: May 3, 2022

Related U.S. Application Data
(60) Provisional application No. 63/333,293, filed on Apr. 21, 2022.
(60) Provisional application No. 63/183,976, filed on May 4, 2021.

(51) Int. Cl. C07K 16/40; C07K 16/28

(52) U.S. Cl. CPC C07K 16/40; C07K 16/2863; C07K 2317/31; C07K 2317/622; C07K 2317/92; A61K 39/00

(57) ABSTRACT

Multispecific binding molecules (MBMs) comprising at least three antigen binding sites that bind to FGR1c, the GH1 domain of Klotho beta (“KLB”), and the GH2 domain of KLB, pharmaceutical compositions containing the MBMs, methods of using the MBMs and pharmaceutical compositions for treating metabolic diseases, nucleic acids encoding the MBMs, cells engineered to express the MBMs, and methods of producing MBMs.

### 1. CROSS-REFERENCE TO RELATED APPLICATIONS

This application claims the priority benefit of U.S. provisional application No. 63/183,976, filed May 4, 2021 and U.S. provisional application No. 63/333,293, filed Apr. 21, 2022, the contents of each of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on Apr. 28, 2022, is named RGN-004US_SL.txt and is 103,195 bytes in size.

### 3. BACKGROUND

Fibroblast growth factor 21 (FGF21) is a protein highly synthesized in the liver that exerts paracrine and endocrine control of many aspects of energy homeostasis in multiple tissues. FGF21 acts on a cell surface receptor complex comprised of two proteins: an FGF receptor (FGFR) and a co-receptor protein, named β-Klotho (KLB). FGF21 binds directly to both of these proteins to activate FGFR signaling activity (Kuro-O, 2018, Nature 552:409-410; Lee et al., 2018, Nature 553:501-505).

FGF receptors are single-pass transmembrane receptor proteins with three extracellular immunoglobulin-type domains (D1-D3) and an intracellular tyrosine kinase domain. KLB is a type-I membrane protein composed of a signal sequence, a large extracellular ligand-binding region, a single transmembrane domain, and a small cytoplasmic region (Kuro-O, 2012, Adv Exp Med Biol 728:25-40). The extracellular ligand-binding region of KLB is composed of tandem repeats, designated GH1 and GH2, with amino acid sequences similar to glycoside hydrolase family 1 enzymes, so-called sugar-cutting enzymes, and binds to the C-terminal tail of FGF21 (Lee et al., 2018, Nature 553:501-505).

In vitro, FGF21 can act through KLB complexed with any of the FGFR1c, FGFR2c, and FGFR3c isoforms. However, gene knockout (KO) analyses and studies with activating antibodies specific for either FGFR1 or the FGFR1/KLB complex suggest that FGFR1c may be particularly important for FGF21's actions in vivo (Adams et al., 2012, Molecular Metabolism 2:31-37; Foltz et al., 2012, Science Translational Medicine 4:162ra153; Kolumam et al., 2015, EBioMedicine 2:730-743; Lan et al., 2017, Cell Metabolism 26:709-718; Wu et al., 2011, Science Translational Medicine 3:113ra126).

In preclinical models of obesity and type 2 diabetes, treatment with FGF21 improves glucose homeostasis and promotes weight loss, and, as a result, FGF21 has attracted considerable attention as a therapeutic agent for the treatment of metabolic syndrome in humans (see, e.g., Lewis et al., 2019, Trends in Endocrinology & Metabolism 30:491-504).

Engineered FGF21 analogs, at pharmacological levels, show considerable improvement in the metabolic syndrome phenotype in animal models. However, only some of these effects (reduced dyslipidemia and body weight) are evident in humans (see, e.g., Zhang et al., 2015, Frontiers in Endocrinology 6:168). To address these shortcomings, bispecific antibodies that bind to KLB and FGFR1 have been generated as alternative FGF21 agonists (see, e.g., Kolumam et al., 2015, EBioMedicine 2(7):730-743; U.S. Pat. No. 9,884,919; Smith et al., 2013, PLoS One 8:e61432). However, as demonstrated herein, bispecific antibodies possess only a fraction of the agonist activity of FGF21.

Accordingly, there is a need in the art with more effective FGF21 agonists. The present disclosure addresses this need and others in the art.

### 4. SUMMARY

The present disclosure provides multispecific binding molecules (“MBMs”) containing at least three antigen-binding sites (“ABS”), the first of which (“ABS1”) binds to FGFR1c, the second of which (“ABS2”) binds to the GH2 domain of KLB, and the third of which (“ABS3”) binds to the GH2 domain of KLB. Without being bound by theory, it is believed that the inclusion of two antigen-binding sites against KLB, one against the GH1 domain and another against the GH2 domain, in addition to an FGFR1c antigen-binding site in an MBM gives rise to a KLB-FGFR1c-MBM complex whose stoichiometry results in greater agonism of FGFR1c than can be achieved by a bispecific antibody. For example, MBMs can in some embodiments having a lower KD for binding to a target molecule and/or have more potent EC50 values in a cell based binding assay than a corresponding parental monospecific antibody or bispecific antibody (e.g., as described in Section 7.5). Exemplary MBMs of the disclosure are described in Section 6.2 and specific embodiments 181 to 326, infra.

The disclosure further provides nucleic acids encoding the MBMs of the disclosure. The nucleic acids encoding the MBMs can be a single nucleic acid (e.g., a vector encoding all polypeptide chains of a MBM) or a plurality of nucleic acids (e.g., two or more vectors encoding the different polypeptide chains of a MBM). The disclosure further provides host cells and cell lines engineered to express the nucleic acids and MBMs of the disclosure. The disclosure further provides methods of producing a MBM of the disclosure. Exemplary nucleic acids, host cells, cell lines, and methods of producing a MBM are described in Section 6.4 and specific embodiments 348 and 353, infra.

The disclosure further provides pharmaceutical compositions comprising the MBMs of the disclosure. Exemplary pharmaceutical compositions are described in Section 6.5 and specific embodiment 327, infra.

Further provided herein are methods of using the MBMs and the pharmaceutical compositions of the disclosure, e.g., for treating a metabolic condition and/or improving metabolism. Exemplary methods are described in Section 6.6 and specific embodiments 1 to 180 and 328 to 347, infra. In some aspects, the methods utilize an MBM as described in Section 6.2 and specific embodiments 181 to 326.

### 5. BRIEF DESCRIPTION OF THE FIGURES

FIG. 1: Schematic representation of metabolic pathways regulated by FGF21, a member of the FGF family that acts as an endocrine hormone.

FIG. 2: Schematic representation of novel KLB and FGFR1c binders 22414, 22401 and 22393, which bind to the GH1 domain of KLB; 22532, which binds to the GH2 domain of KLB; and ADI-19842, which binds to the D3 domain of FGFR1c.

FIG. 3: Schematic representation the domains in the FGFR1 receptor/co-receptor complex of FGFR1c/KLB bound by the bispecific binding molecules (BBMs) REGN4355, REGN 4366, REGN 4370, REGN 4376 and REGN 4304. REGN4304 targets the FGFR1c D2 and KLB GH2 while the remaining bispecific binding molecules target the FGFR1c D3 and KLB GH1 domains.

FIG. 4: Graph illustrating the modest activation by bispecific binding molecules (BBMs) REGN4366 and REGN4304 in HEK293/SRE-luc/hFGFR1c/hKLB cells as compared to FGFR21.

FIG. 5: FIG. 5 shows exemplary configurations of trispecific binding molecules containing three antigen binding moieties (indicated as “1”, “2”, “3”). Clockwise from top left: trispecific variant containing an N-terminal scFv domain (the 2+1 N-scFv configuration); trispecific variant containing a C-terminal scFv domain (the 2+1 C-scFv configuration); trispecific variant containing a C-terminal Fab domain (the 2+1 C-Fab configuration); trispecific variant containing an N-terminal Fab domain (the 2+1 N-Fab configuration). The three antigen binding moieties together have three antigen binding sites that bind, in no particular order, to the GH1 domain of KLB, the GH2 domain of KLB, and FGFR1c (e.g., in the D1, D2 or D3 domain).

FIGS. 6A-6B: FIG. 6A shows trispecific variants of REGN4366, which is a bispecific binding molecule targeting the GH1 domain of KLB and the D3 domain of FGFR1c, created by the addition of a GH2 binding arm at different locations in the molecule. Clockwise from top left: trispecific variant containing an N-terminal scFv domain (the 2+1 N-scFv configuration); trispecific variant containing a C-terminal scFv domain (the 2+1 C-scFv configuration); trispecific variant containing a C-terminal Fab domain (the 2+1 C-Fab configuration); trispecific variant containing an N-terminal Fab domain (the 2+1 N-Fab configuration). FIG. 6B: Bar graph showing activity in relation to linker length variation.

FIG. 7A-7B: FIG. 7A is a graph illustrating enhanced activity of F1K_scFv6 and F1K_Fab6 compared to parental REGN4366 as well as to RGN4304 in a HEK293.SRElluc.hFGFR1c.hKLB cell reporter assay. Filled circle in FIG. 7A depict data points for human FGF21 as the positive control. FIG. 7B is a schematic representation of targeting the GH2 domain and how this correlates to a better agonism.

FIG. 8A-8B: FIG. 8A is schematic representation of a screen for variants of the 2+1 N-scFv format, including linker variants (I); an isotype variant (II); a variant with alternative GH2-binding sequence (III); and variants with alternative GH1 binding sequence. FIG. 8A discloses SEQ ID NOS: 55, 24, 73, 57, 74-75 and 44, respectively, in order of appearance. FIG. 8B is schematic representation of a screen for arm arrangement, distance, and orientation variants of the 2+1 N-scFv format.

FIG. 9: FIG. 9 shows the results of the study evaluating six linker length variants of the molecule designated scFv6 (containing the GH1 binder designated 22393 (or 393) at the (1) position, the FGFR1 binder designated ADI-19842 or 842 at the (2) position, and the GH2 binder designated 22532 (or 532) in the scFv format at the (3) position). The molecules contained linkers from 7 to 45 amino acids between the FGFR1 binding domain and the N-terminal 532 scFv domain component. The scFvs were configured in the order VL-VH, and the linker designations “L20H7”, “L20H15”, “L20H22”, “L20H30”, “L20H37”, and “L20H45” refer to a 20-amino acid linker separating the VL and the VH of the scFv and a linker of 7, 15, 22, 30, 37 or 45 amino acids at the C-terminus of the scFv, separating the scFv from the adjoining VH. Trispecific binding molecules of all linker lengths exhibit greater activity than REGN4304, the control bispecific binding molecule.

FIGS. 10A-10B: F1K_scFv6 (30 aa linker) and scFv6_LK7 (7aa linker) strongly activate FGFR1c signaling in HEK293 cells stably expressing hFGFR1c and hKLB. FIG. 10A: Western blot illustrating drug concentration-dependent FGFR1c signaling through ERK and PLCγ phosphorylation as a result of serum starvation for 16 hrs followed by drug treatment for 15 min at concentrations of 1 nM and 10 nM. FIG. 10B: Western blot illustrating time-dependent FGFR1c signaling through ERK and PLCγ phosphorylation as a result of serum starvation for 16 hrs followed by drug treatment at a concentration of 10 nM for 15, 30 min and 1, 2, 4 and 6-hr incubation periods. Unless specified otherwise, the term “F1K_scFv6” without a linker length suffix in FIGS. 10A-10B and elsewhere in the specification refers to a molecule with a 30-amino acid linker between the scFv domain (ABS3 in FIG. 5) and the Fab domain and is sometimes referred to as F1K_scFv6-LK30.

FIGS. 11A-11C: F1K_scFv6 and Fab6 activate the ERK pathway in primary human adipocytes. FIG. 11A: Western blot illustrating FGFR1c signaling in primary human adipocytes through ERK and PLCγ phosphorylation. Adipocytes were differentiated for 8 days then serum starved for 4 hrs and treated with drugs for 15 minutes at a concentration of 10 nM. FIGS. 11B and 11C: Graph illustrating enhanced ERK activity using a FRET-based p-ERK immuno-capture assay of F1K_scFv6 and F1K_Fab6 relative to REGN1945 and REGN4366. Differentiated human subcutaneous adipocytes were cultured for one day for recovery then serum starved for 4 hrs and treated with drugs for 15-60 minutes. Unless specified otherwise, the term “F1K_scFv6” without a linker length suffix in FIGS. 11A-11C and elsewhere in the specification refers to a molecule with a 30-amino acid linker between the scFv domain (ABS3 in FIG. 5) and the Fab domain and is sometimes referred to as F1K_scFv6-LK30. Unless specified otherwise, the term “F1K_Fab6” without a linker length suffix in FIGS. 11B-11C and elsewhere in the specification refers to a molecule with a 30-amino acid linker between the Fab domain of ABS3 (as illustrated as “3” in FIG. 5) and the Fc domain and is sometimes referred to as F1K_Fab6-LK30.

FIGS. 12A-12B: Schematic representation of how clusters of FGFR1c, KLB and FGF21 form active complexes (FIG. 12A) and the potential stoichiometric complexes formed between FGFR1c and KLB receptors and the trispecifics F1K_scFv6 or F1K_Fab6 compared to bispecifics and monospecific controls (FIG. 12B). Unless specified otherwise, the term “F1K_scFv6” without a suffix in FIG. 12B and elsewhere in the specification refers to a molecule with a 30-amino acid linker between the scFv domain (ABS3 in FIG. 5) and the Fab domain and is sometimes referred to as F1K_scFv6-LK30. Unless specified otherwise, the term “F1K_Fab6” without a suffix in FIG. 12B and elsewhere in the specification refers to a molecule with a 30-amino acid linker between the Fab domain of ABS3 (as illustrated as “3” in FIG. 5) and the Fc domain and is sometimes referred to as F1K_Fab6-LK30.

FIGS. 13A-130: Monospecific (anti-KLB; REGN4661) and bispecific binding molecules (anti-KLB×FGFR1c; REGN4304) bind KLB/FGFR1c with different stoichiometries compared to trispecific mAbs (F1K_scFv6 IgG1 and F1K_Fab6 IgG1). FIG. 13A: REGN4661:KLB complexes (solid line) were analyzed by asymmetric flow field-flow fractionation coupled to multi-angle light scattering (A4F-MALS). Fractograms from individual samples of REGN4661 (dashed line) and KLB (dotted line) are also overlaid. Relative UV absorbance at 215 nm as a function of retention time is shown for each sample and the measured molar masses of resolved peaks are indicated. FIG. 13B: REGN4303:KLB complexes (thick solid line) and REGN4303:KLB:FGFR1c complexes (thin solid line) were analyzed by asymmetric flow field-flow fractionation coupled to multi-angle light scattering (A4F-MALS). Fractograms from individual samples of REGN4303 (dashed line), KLB (dotted line) and FGFR1c (grey dotted line) are also overlaid. Relative UV absorbance at 215 nm as a function of retention time is shown for each sample and the measured molar masses of resolved peaks are indicated. FIG. 13C: F1K_scFv6 IgG1:KLB complexes (thick solid line) and F1K_scFv6 IgG1:KLB:FGFR1c complexes (0.2 μM:0.2 μM:0.2 μM, thin solid line) were analyzed by asymmetric flow field-flow fractionation coupled to multi-angle light scattering (A4F-MALS). Relative UV absorbance at 215 nm as a function of retention time is shown for each sample and the measured molar masses of resolved peaks are indicated. FIG. 13D: F1K_Fab6 IgG1:KLB complexes (thick solid line) and F1K_Fab6 IgG1:KLB:FGFR1c complexes (0.2 μM:0.2 μM:0.2 μM, thin solid line) were analyzed by asymmetric flow field-flow fractionation coupled to multi-angle light scattering (A4F-MALS). Relative UV absorbance at 215 nm as a function of retention time is shown for each sample and the measured molar masses of resolved peaks are indicated. Unless specified otherwise, the term “F1K_scFv6” without a suffix in FIG. 13C and elsewhere in the specification refers to a molecule with a 30-amino acid linker between the scFv domain (ABS3 in FIG. 5) and the Fab domain and is sometimes referred to as F1K_scFv6-LK30. Unless specified otherwise, the term “F1K_Fab6” without a suffix in FIG. 13D and elsewhere in the specification refers to a molecule with a 30-amino acid linker between the Fab domain of ABS3 (as illustrated as “3” in FIG. 5) and the Fc domain and is sometimes referred to as F1K_Fab6-LK30.

FIG. 14: FIG. 14 depicts the wild-type sequence of the heavy chain constant region of human IgG1 (human IGHG1 heavy chain constant region; UniProt Accession No. P01857). CH1=amino acids 1-98; upper hinge=amino acids 99-108; core hinge=109-112; lower hinge=113-121; CH2=120-223; CH3=224-330. Indicated amino acid numbering is relative to the depicted sequence. The upper, core and lower hinge regions are boxed. As shown in the figure, the last two amino acids of the lower hinge correspond to the first two amino acids of the CH2 domain. FIG. 14 discloses SEQ ID NO: 76.

FIG. 15: FIG. 15 depicts the wild-type sequence of the heavy chain constant region of human IgG2 (human IGHG2 heavy chain constant region; UniProt Accession No. P01859). CH1=amino acids 1-98; upper hinge=amino acids 99-105; core hinge=106-109; lower hinge=110-117; CH2=116-219; CH3=220-326. Indicated amino acid numbering is relative to the depicted sequence. As shown in the figure, the last two amino acids of the lower hinge correspond to the first two amino acids of the CH2 domain. FIG. 15 discloses SEQ ID NO: 77.

FIG. 16: FIG. 16 depicts the wild-type sequence of the heavy chain constant region of human IgG4 (human IGHG4 heavy chain constant region; UniProt Accession No. P01861). CH1=amino acids 1-98; upper hinge=amino acids 99-105; core hinge=106-109; lower hinge=110-118; CH2=117-220; CH3=221-227. Indicated amino acid numbering is relative to the depicted sequence. As shown in the figure, the last two amino acids of the lower hinge correspond to the first two amino acids of the CH2 domain. FIG. 16 discloses SEQ ID NO: 78.

FIG. 17: FIG. 17 depicts an amino acid sequence alignment of the upper hinge, core hinge, lower hinge, CH2, and CH3 of the noted chimeric IgG heavy chain constant domain constructs. Indicated amino acid numbering is EU numbering. Shaded cells of the lower hinge indicate amino acids also corresponding to the first to amino acids of the CH2 domain.

FIG. 18: FIG. 18 depicts representative data demonstrating antibody titers of the depicted F1K_scFv6 linker length variants comprising hetero-dimers of either IgG4 S108P/IgG4 S108P Star (H315R, Y316F) or IgG1 PVA/IgG1 PVA Star (H315R, Y316F) following stable expression in Chinese hamster ovary (CHO) cells. Linkers of varying lengths between the Fab and scFv were tested.

FIGS. 19A-19G: FIGS. 19A-19G depict representative enzyme-linked immunosorbent assay (ELISA) data demonstrating binding of the noted controls and antibodies to hFCRγ1 (FIG. 19A); hFCRγ2A (H131) (FIG. 19B); hFCRγ2A (R131) (FIG. 19C); hFCRγ2B (FIG. 19D); hFCRγ3A (V158) (FIG. 19E); hFCRγ3A (F158) (FIG. 19F); and hFCRγ3B (FIG. 19G). Descriptions of the control and test antibodies are provided in Table 7.

FIG. 20: FIG. 20 depicts representative results from a surrogate antibody dependent cell-mediated cytotoxicity (ADCC) assay in which the indicated F1K_Fab6 variants with differing Fc regions were tested, along with controls.

FIG. 21: FIG. 21 depicts representative results from a surrogate ADCC assay in which the indicated F1K_Fab6 variants with differing Fc regions were tested, along with controls.

FIG. 22: FIG. 22 depicts representative results from a luciferase reporter assay demonstrating F1K_scFv6 variants with differing Fc regions along with controls caused activation of HEK293.SREluc.hFGFR1c.hKLB cells.

FIG. 23: FIG. 23 depicts representative results from a luciferase reporter assay demonstrating F1K_Fab6 variants with differing Fc regions and linker lengths along with controls caused activation of HEK293.SREluc.hFGFR1c.hKLB cells.

FIG. 24: FIG. 24 depicts representative results from phospho-ERK activation assay demonstrating F1K_scFv6 and Fab6 constructs with either with IgG4 S108P or IgG1 PVA Fc regions or His.hFGF21 caused activation in primary human adipocytes.

### 6. DETAILED DESCRIPTION

### 6.1. Definitions

As used herein, the following terms are intended to have the following meanings:

Antigen Binding Site or ABS: The term “antigen binding site” or “ABS” as used herein refers to the portion of a MBM that is capable of specific, non-covalent, and reversible binding to a target molecule. The MBMs of the disclosure comprise a first ABS (“ABS1”), a second ABS (“ABS2”), and a third ABS (“ABS3”).

Associated: The term “associated” in the context of an MBM refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional MBM in which ABS1, ABS2 and ABS3 can bind their respective targets. Examples of associations that might be present in an MBM of the disclosure include (but are not limited to) associations between homodimeric or heterodimeric Fc domains in an Fc region, associations between VH and VL regions in a Fab or scFv, associations between CH1 and CL in a Fab, and associations between CH3 and CH3 in a domain substituted Fab.

Complementarity Determining Reaction or CDR: The terms “complementarity determining region” or “CDR,” as used herein, refer to the sequences of amino acids within antibody variable regions which confer antigen specificity and binding affinity. In general, there are three CDRs in each heavy chain variable region (CDR-H1, CDR-H2, HCDR-H3) and three CDRs in each light chain variable region (CDR1-L1, CDR-L2, CDR-L3). Exemplary conventions that can be used to identify the boundaries of CDRs include, e.g., the Kabat definition, the Chothia definition, the ABS definition and the IMGT definition. See, e.g., Kabat, 1991, “Sequences of Proteins of Immunological Interest,” National Institutes of Health, Bethesda, Md. (Kabat numbering scheme); Al-Lazikani et al., 1997, J. Mol. Biol. 273:927-948 (Chothia numbering scheme); Martin et al., 1989, Proc. Natl. Acad. Sci. USA 86:9268-9272 (ABS numbering scheme); and Lefranc et al., 2003, Dev. Comp. Immunol. 27:55-77 (IMGT numbering scheme). Public databases are also available for identifying CDR sequences within an antibody.

Derived from: As used herein, the term “derived from” indicates a relationship between a first and a second molecule. It generally refers to structural similarity between the first molecule and a second molecule and does not connote or include a process or source limitation on a first molecule that is derived from a second molecule.

EC50: The term “EC50” refers to the half maximal effective concentration of an antibody or MBM which induces a response halfway between the baseline and maximum after a specified exposure time. The EC50 essentially represents the concentration of an antibody or MBM where 50% of its maximal effect is observed. In certain embodiments, the EC50 value equals the concentration of an antibody or MBM that gives half-maximal binding to cells expressing the target molecules that can be specifically bound by an antibody or MBM, e.g., as determined by FACS binding assay. Thus, reduced or weaker binding is observed with an increased EC50, or half maximal effective concentration value. EC50 values of MBMs of the disclosure can in some embodiments be characterized by EC50 values of about 10⁻⁵M or less (e.g., less than 10⁻⁵M, less than 10⁻⁶M, less than 10⁻⁷M, less than 10⁻⁸M, or less than 10⁻⁹M).

Epitope: An epitope, or antigenic determinant, is a portion of an antigen (e.g., target molecule) recognized by an antibody or other antigen-binding moiety as described herein. An epitope can be linear or conformational.

Fab: The term “Fab” in the context of an MBM of the disclosure refers to a pair of polypeptide chains, the first comprising a variable heavy (VH) domain of an antibody N-terminal to a first constant domain (referred to herein as C1), and the second comprising variable light (VL) domain of an antibody N-terminal to a second constant domain (referred to herein as C2) capable of pairing with the first constant domain. In a native antibody, the VH is N-terminal to the first constant domain (CH1) of the heavy chain and the VL is N-terminal to the constant domain of the light chain (CL). The Fabs of the disclosure can be arranged according to the native orientation or include domain substitutions or swaps on that facilitate correct VH and VL pairings, particularly where the MBMs of the disclosure comprise non-identical Fabs. For example, it is possible to replace the CH1 and CL domain pair in a Fab with a CH3-domain pair to facilitate correct modified Fab-chain pairing in heterodimeric MBMs. It is also possible to reverse CH1 and CL, so that the CH1 is attached to VL and CL is attached to the VH, a configuration generally known as Crossmab. Alternatively, or in addition to, the use of substituted or swapped constant domains, correct chain pairing can be achieved by the use of universal light chains that can pair with both variable regions of a heterodimeric MBM of the disclosure.

FGF Receptor 1c and FGFR1c: The terms “FGF receptor 1c,” “FGFR1c” and similar terms refer to any native fibroblast growth factor receptor 1c (FGFR1c) from any vertebrate source, including mammals such as primates (e.g., humans, cynomolgus monkey (cyno)), dogs, and rodents (e.g., mice and rats), unless otherwise indicated. The term encompasses “full-length,” unprocessed FGFR1c as well as any form of FGFR1c those results from processing in the cell. The term also encompasses naturally occurring variants of FGFR1c, e.g., splice variants or allelic variants. The amino acid sequence of an exemplary human FGFR1c is:

(SEQ ID NO: 1)
MWSWKCLLEWAVLVTATLCTARPSPTLPEQAQPWGAPVEVESELVHPGDL
LQLRCRLRDDVQSINWLRDGVQLAESNRTRITGEEVEVQDSVPADSGLYA
CVTSSPSGSDTTYFSVNVSDALPSSEDDDDDDDSSSEEKETDNTKPNPVA
PYWTSPEKMEKKLHAVPAAKTVKFKCPSSGTPNPTLRWLKNGKEFKPDRI
GGYKVRYATWSIIMDSVVPSDKGNYTCIVENEYGSINHTYQLDVVERSPH
RPILQAGLPANKTVALGSNVEFMCKVYSDPQPHIQWLKHIEVNGSKIGPD
NLPYVQILKTAGYNTTDKEMEVLHLRNVSFEDAGEYTCLAGNSIGLSHHS
AWLTVLEALEERPAVIVITSPLYLEIIIYCTGAFLISCMVGSVIVYKMKS
GTKKSDFHSQMAVEIKLAKSIPLRRQVTVSADSSASMNSGVLLVRPSRLS
SSGTPMLAGVSEYELPEDPRWELPRDRLVLGKPLGEGCFGQVVLAEAIGL
DKDKPNRVTKVAVKMLKSDATEKDLSDLISEMEMMKMIGKEIKNIINLLG
ACTQDGPLYVIVEYASKGNLREYLQARRPPGLEYCYNPSHNPEEQLSSKD
LVSCAYQVARGMEYLASKKCIHRDLAARNVLVTEDNVMKIADFGLARDIH
HIDYYKKTTNGRLPVKWMAPEALFDRIYTHQSDVWSFGVLLWEIFTLGGS
PYPGVPVEELFKLLKEGHRMDKPSNCTNELYMMMRDCWHAVPSQRPTFKQ
LVEDLDRIVALTSNQEYLDLSMPLDQYSPSFPDTRSSTCSSGEDSVFSHE
PLPEEPCLPRHPAQLANGGLKRR

Half Antibody: The term “half antibody” refers to a molecule that comprises at least one ABS or ABS chain (e.g., one chain of a Fab) and can associate with another molecule comprising an ABS or ABS chain through, e.g., a disulfide bridge or molecular interactions (e.g., knob-in-hole interactions between Fc heterodimers). A half antibody can be composed of one polypeptide chain or more than one polypeptide chains (e.g., the two polypeptide chains of a Fab). In a preferred embodiment, a half-antibody comprises an Fc domain.

Host cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey or human cell line, for example HKB11 cells, PER.C6 cells, HEK cells or CHO cells.

Beta (β) klotho, klotho beta, and KLB: The terms “beta (β) klotho,” “klotho beta,” “KLB” and similar terms refers to a polypeptide or any native beta klotho from any vertebrate source, including mammals such as primates (e.g., humans, cynomolgus monkey (cyno)), dogs, and rodents (e.g., mice and rats), unless otherwise indicated, and, in certain embodiments, included related beta klotho polypeptides, including SNP variants thereof. Beta klotho comprises two domains, beta klotho 1 (KLB1) and beta klotho 2 (KLB2). Each beta klotho domain comprises a glycosyl hydrolase region. For example, the KLB1 domain of human beta klotho comprises amino acid residues 1-508 with the first glycosyl hydrolase region (referred to herein as GH1) comprising amino acid residues 77-508, and the KLB2 domain of human beta klotho comprises amino acid residues 509-1044 with the second glycosyl hydrolase region (referred to herein as GH2) comprising amino acid residues 517-967. The terms “beta (β) klotho,” “klotho beta” and “KLB” encompass “full-length,” unprocessed KLB as well as any form of KLB that results from processing in the cell. The term also encompasses naturally occurring variants of KLB, e.g., splice variants or allelic variants. The amino acid sequence of an exemplary human KLB is:

(SEQ ID NO: 2)
FSGDGRAIWSKNPNETPVNESQLELYDTFPKNFFWGIGTGALQVEGSWKK
DGKGPSIWDEIFIHTEILKNVSSTNGSSDSYIFLEKDLSALDFIGVSFYQ
FSISWPRLFPDGIVTVANAKGLQYYSTLLDALVLRNIEPIVTLYHWDLPL
ALQEKYGGWKNDTIIDIFNDYATYCFQMFGDRVKYWITIEINPYLVAWHG
YGTGMHAPGEKGNLAAVYTVGHNLIKAHSKVWEINYNTEIFRPHQKGWLS
ITLGSHWIEPNRSENTMDIFKCQQSMVSVLGWFANPIHGDGDYPEGMRKK
LFSVLPIFSEAEKEIEMRGTADFFAFSFGPNNFKPLNTMAKMGQNVSLNL
REALNWIKLEYNNPRILIAENGWFTDSRVKTEDTTAIYMMKNFLSQVLQA
IRLDEIRVFGYTAWSLLDGFEWQDAYTIRRGLFYVDFNSKQKERKPKSSA
HYYKQIIRENGFSLKESTPDVQGQFPCDFSWGVTESVLKPESVASSPQFS
DPEILYVWNATGNRLLEIRVEGVRLKTRPAQCTDFVNIKKQLEMLARMKV
THYRFALDWASVLPTGNLSAVNRQALRYYRCVVSEGLKLGISAMVTLYYP
THAEILGLPEPLLHADGWLNPSTAEAFQAYAGLCFQELGDLVKLWITINE
PNRLSDIYNRSGNDTYGAAEINLLVAHALAWRLYDRQFRPSQRGAVSLSL
HADWAEPANPYADSHWRAAERFLQFEIAWFAEPLFKTGDYPAAMREYIAS
KEIRRGLSSSALPRLTEAERRLLKGTVDFCALNEIFTTRFVMHEQLAGSR
YDSDRDIQFLQDITRLSSPTRLAVIPWGVRKLLRWVRRNYGDMDIYITAS
GIDDQALEDDRLRKYYLGKYLQEVLKAYLIDKVRIKGYYAFKLAEEKSKP
RFGFFTSDFKAKSSIQFYNKVISSRGFPFENSSSRCSQTQENTECTVCLF
LVQKKPLIFLGCCFFSTLVLLLSIAIFQRQKRRKFWKAKNLQHIPLKKGK
RVVS

Metabolic Condition: The term “metabolic condition” as used herein refers to metabolic disorders as well as situations in which a metabolic indicator (e.g., weight or body mass index, HDL cholesterol, LDL cholesterol, blood triglycerides, blood glucose) is outside a range generally accepted as normal or healthy be a medical professional. Examples of metabolic disorders include metabolic syndrome, obesity, fatty liver, hyperinsulinemia, type 2 diabetes, nonalcoholic steatohepatitis (“NASH”), nonalcoholic fatty liver disease (“NAFLD”), hypercholesterolemia, and hyperglycemia.

Multispecific Binding Molecule or MBM: The term “multispecific binding molecule” or “MBM” as used herein refers to molecules (e.g., assemblies of multiple polypeptide chains) comprising two half antibodies and which specifically bind to at least two different epitopes (and in some instances three or more different epitopes) and comprise an ABS1, and ABS2, and an ABS3.

Operably linked: The term “operably linked” as used herein refers to a functional relationship between two or more regions of a polypeptide chain in which the two or more regions are linked so as to produce a functional polypeptide.

Peptide, polypeptide and protein: The terms “peptide,” “polypeptide,” and “protein” are used interchangeably herein and refer to a molecule or compound comprising amino acid residues covalently linked by peptide bonds. A protein, polypeptide or peptide must contain at least two amino acids, and no limitation is placed on the maximum number of amino acids in the molecule or compound. Thus, these terms refer to both short chains, which also commonly are referred to in the art as peptides, oligopeptides and oligomers, for example, and to longer chains, which generally are referred to in the art as proteins or polypeptides, of which there are many types.

Simile Chain Fv or scFv: The term “single chain Fv” or “scFv” as used herein refers to a polypeptide chain comprising the VH and VL domains of antibody, where these domains are present in a single polypeptide chain.

Specifically (or selectively) binds: The term “specifically (or selectively) binds” as used herein means that a MBM or antigen binding site (“ABS”) thereof forms a complex with a target molecule (e.g., KLB or FGFR1c) that is relatively stable under physiologic conditions. Specific binding can be characterized by a KD of about 5×10⁻²M or less (e.g., less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, or less than 10⁻¹⁰M). Methods for determining the binding affinity of an antibody or an antibody fragment, e.g., an MBM or ABS, to a target molecule are well known in the art and include, for example, equilibrium dialysis, surface plasmon resonance (e.g., Biacore assays), fluorescent-activated cell sorting (FACS) binding assays and the like. A MBM or ABS thereof antibody that specifically binds a target molecule from one species can, however, have cross-reactivity to the target molecule from one or more other species.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Tetravalent: The term “tetravalent” as used herein refers to refers to a MBM that has four antigen binding sites, e.g., ABS1, ABS2 and ABS3, and a fourth antigen binding site (ABS4). Generally, the four antigen binding sites can bind to the same epitope or different epitopes, but in preferred embodiments of the MBMs of the disclosure, ABS1, ABS2 and ABS3 are FGR1c, GH1 and GH2 binding sites and ABS4 can be an FGR1c, GH1, GH2 or other binding site. In some embodiments, a tetravalent MBM is trispecific and binds only to FGFR1c, GH1 and GH2.

Treat, Treatment, Treating: As used herein, the terms “treat”, “treatment” and “treating” refer to the reduction or amelioration of the progression, severity and/or duration of a metabolic condition, or the amelioration of one or more symptoms (preferably, one or more discernible symptoms) of a metabolic condition resulting from the administration of one or more MBMs of the disclosure. In specific embodiments, the terms “treat”, “treatment” and “treating” refer to the amelioration of at least one measurable physical parameter of a metabolic condition, not necessarily discernible by the patient, such as a reduction in weight, a reduction in circulating HGL cholesterol, an increase in circulating LDL cholesterol, a reduction in blood triglycerides, and a reduction in blood glucose. A reduction in weight, a reduction in circulating HGL cholesterol, an increase in circulating LDL cholesterol, a reduction in blood triglycerides, and a reduction in blood glucose are considered to be an improvement in metabolism. In other embodiments the terms “treat”, “treatment” and “treating” refer to the inhibition of the progression of a metabolic condition, either physically by, e.g., stabilization of a discernible symptom, physiologically by, e.g., stabilization of a physical parameter, or both. In other embodiments the terms “treat”, “treatment” and “treating” refer to the stabilization of the metabolic condition. The MBMs and pharmaceutical compositions of the disclosure can be administered to a subject in amounts effective to treat a metabolic condition and/or improve metabolism in the subject.

Trispecific binding molecule: The term “trispecific binding molecules” or “TBMs” as used herein refers to molecules that specifically bind to three epitopes and comprise three or more antigen binding sites. The TBMs of the disclosure bind to FGFR1c, GH1 and GH2. The antigen-binding sites can each independently be an antibody fragment (e.g., scFv, Fab, nanobody) or a non-antibody derived binder (e.g., fibronectin, Fynomer, DARPin).

Trivalent: The term “trivalent” as used herein refers to refers to a MBM that has three antigen binding sites, e.g., ABS1, ABS2 and ABS3. Generally, the three antigen binding sites can bind to the same epitope or different epitopes, but in preferred embodiments of the MBMs of the disclosure, the three antigen binding sites comprises a GH1 antigen binding site, a GH2 antigen binding site and a GFGR1c antigen binding site.

Universal Light Chain: The term “universal light chain” as used herein in the context of a MBM refers to a light chain polypeptide capable of pairing with the heavy chain region of Fab1 to form Fab1 and capable of pairing with the heavy chain region of Fab2 to form Fab2. Universal light chains are also known as “common light chains.”

VH: The term “VH” refers to the variable region of an immunoglobulin heavy chain of an antibody, including the heavy chain of an scFv or a Fab.

VL: The term “VL” refers to the variable region of an immunoglobulin light chain, including the light chain of an scFv or a Fab.

Fc Domain and Fc Reaction: The term “Fc domain” refers to a portion of the heavy chain that pairs with the corresponding portion of another heavy chain. The term “Fc region” refers to the region of antibody-based binding molecules formed by association of two heavy chain Fc domains. The two Fc domains within the Fc region may be the same or different from one another. In a native antibody the Fc domains are typically identical, but for the purpose of producing the MBMs of the disclosure, one or both Fc domains might advantageously be modified to allow for heterodimerization.

### 6.2. Multispecific Binding Molecules (MBMs)

### 6.2.1. KLB and FGFR1c ABSs

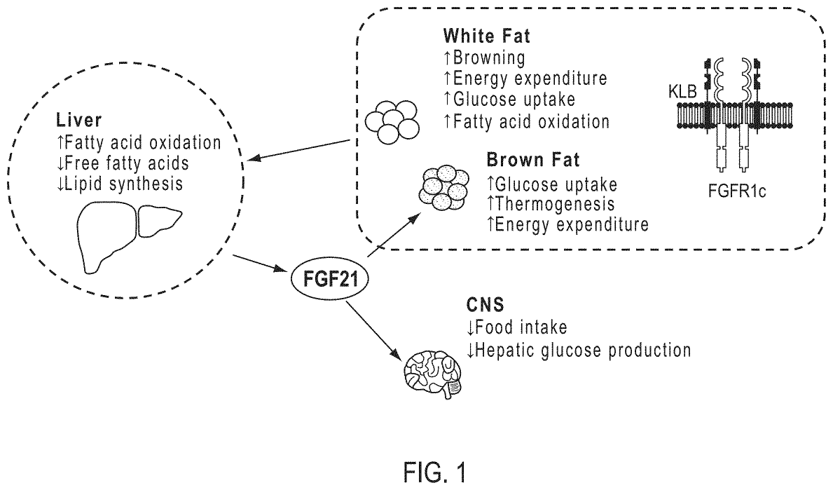

MBMs of the disclosure contain an ABS1 that binds to FGFR1c, an ABS2 that binds to the GH2 domain of KLB, and an ABS3 that binds to the GH2 domain of KLB. Without being bound by theory, it is believed that the binding of an MBM with these three binding domains agonizes the receptor complexes and results in the metabolic benefits illustrated in FIG. 1. ABS1, ABS2 and ABS3 can be derived from one or more suitable anti FGFR1c, anti-GH1 domain and anti-GH2 domain antibodies or non-immunoglobulin based antigen binding sites. An antibody from one or more of ABS1, ABS2 and ABS3 is derived is sometimes referred to herein as a “parental” antibody.

The KLB and FGFR1c parental antibodies can be monoclonal antibodies (e.g., murine or rabbit monoclonal antibodies), chimeric antibodies, humanized antibodies, human antibodies, primatized antibodies, bispecific antibodies, single chain antibodies, etc. In various embodiments, the MBMs of the disclosure comprise all or a portion of a constant region of a parental derived. In some embodiments, the constant region is an isotype selected from: IgA (e.g., IgA1 or IgA2), IgD, IgE, IgG (e.g., IgG1, IgG2, IgG3 or IgG4), and IgM.

The term “monoclonal antibody” as used herein is not limited to antibodies produced through hybridoma technology. A monoclonal antibody is derived from a single clone, including any eukaryotic, prokaryotic, or phage clone, by any means available or known in the art.

Monoclonal antibodies useful as a source of KLB and FGFR1c ABSs can be prepared using a wide variety of techniques known in the art including the use of hybridoma, recombinant, and phage display technologies, or a combination thereof.

The term “chimeric” antibody as used herein refers to an antibody having variable sequences derived from a non-human immunoglobulin, such as a rabbit, rat or a mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397, which are incorporated herein by reference in their entireties.

“Humanized” forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are hereby incorporated by reference in their entireties.

“Human antibodies” include antibodies having the amino acid sequence of a human immunoglobulin and include antibodies isolated from human immunoglobulin libraries or from animals transgenic for one or more human immunoglobulin and that do not express endogenous immunoglobulins. Human antibodies can be made by a variety of methods known in the art including phage display methods using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins but which can express human immunoglobulin genes. See, e.g., PCT publications WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. Fully human antibodies that recognize a selected epitope can be generated using a technique referred to as “guided selection.” In this approach, a selected non-human monoclonal antibody, e.g., a mouse antibody, is used to guide the selection of a completely human antibody recognizing the same epitope (see, Jespers et al., 1988, Biotechnology 12:899-903).

“Primatized antibodies” comprise monkey variable regions and human constant regions. Methods for producing primatized antibodies are known in the art. See, e.g., U.S. Pat. Nos. 5,658,570; 5,681,722; and 5,693,780, which are incorporated herein by reference in their entireties.

In some embodiments, the parental antibodies for the MBMs of the disclosure are generated using VELOCIMMUNE® technology (see, for example, U.S. Pat. No. 6,596,541, Regeneron Pharmaceuticals, VELOCIMMUNE®). High affinity chimeric parental antibodies to FGFR1c, the GH2 domain, GH2 domain or any combination thereof can be initially isolated having human variable regions and mouse constant regions. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Antibodies of interest may also be isolated from mouse B-cells. Briefly, splenocytes are harvested from each mouse and B-cells are sorted (as described in US 2007/0280945A1, for example) by FACS using the antigen of interest as the sorting reagent that binds and identifies reactive antibodies (antigen-positive B cells). Various methods of identifying and sorting antigen positive B cells, as well as constructing immunoglobulin gene expression cassettes by PCR for preparation of cells expressing recombinant antibodies, are well-known in the art. See e.g., WO20141460741, U.S. Pat. No. 7,884,054B2, and Liao, et al., 2009, J Virol Methods 158(1-2):171-9.

Initially, high affinity chimeric antibodies are isolated having a human variable region and a mouse constant region. The antibodies are characterized and selected for desirable characteristics, including affinity, selectivity, epitope, etc. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

Examples of publications disclosing anti-FGFR1c and/or anti-KLB parental antibodies for use in the MBMs of the disclosure include, but are not limited to, U.S. Patent Publication No. US 2015/0218276 and US 2011/0135657; U.S. Pat. Nos. 9,738,716, 9,085,626, 8,263,074; Min et al., 2018, J. Biol. Chem. 293:14678; and Foltz et al., 2012, Sci. Transl. Med. 4:162ra153.

In some embodiments, the FGFR1c binders and FGFR1c binder sequences that can be incorporated into the MBMs of the disclosure are identified in Tables 1A and 1B, respectively. The D1 loop of FGFR1c is absent from some isoforms of FGFR1c due to alternative splicing. Thus, it is preferred that ABS1 bind to loop D2 or loop D3 of FGFR1c.

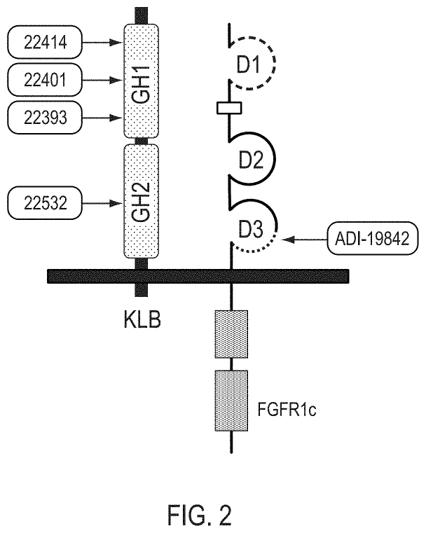

TABLE 1A
FGFR1c Binders

| | | | Sequence in Patent |
|---|---|---|---|
| Name | Epitope | Reference | Reference |
| YW182.5_YGDY | loop D2 | US 2015/0218276 | Heavy chain: Seq ID |
| (Genentech) | | | No. 133; Light chain: |
| | | | Seq ID No. 135 |
| R1Mab1 | loop D2 | U.S. Pat. No. | Heavy chain: Seq ID |
| (Genentech) | | 9,085,626 | No. 2; Light chain: |
| | | | Seq ID No. 6 |
| R1Mab2 | loop D2 | U.S. Pat. No. | Heavy chain: Seq ID |
| (Genentech) | | 9,085,626 | No. 3; Light chain: |
| | | | Seq ID No. 6 |
| R1Mab3 | loop D2 | U.S. Pat. No. | Heavy chain: Seq ID |
| (Genentech) | | 9,085,626 | No. 4; Light chain: |
| | | | Seq ID No. 6 |
| FR1-H7 | loop D2 | U.S. Pat. No. | Heavy chain: FIG. 1A; |
| (ImClone/Eli Lilly) | | 8,263,074 | Light chain: FIG. 1B |
| FR1-A1 | loop D3 | U.S. Pat. No. | Heavy chain: FIG. 2A; |
| (ImClone/Eli Lilly) | | 8,263,074 | Light chain: FIG. 2B |

TABLE 1B
FGFRIc Binders - VH and VL sequences

| | | | SEQ |
|---|---|---|---|
| | | | ID |
| Binder | Chain | Sequence | NO: |
| FR1-H7 | VH | EVQLVQSGAEVKKPGASVKVSCKVSGYTFTDYYMHWVQ | 3 |
| (ImClone/ | | QAPGKGLEWMGLVDPEDGETIYAEKFQGRVTITADTSTDT | |
| Eli Lilly) | | AYMELSSLRSEDTAVYYCARDDYMDVWGKGTLVTVSS | |
| | VL | ETTLTQSPDTLSLSPGEGATLSCRASQSVSGSALAWYQQ | 4 |
| | | KPGQAPRLLIYDASSRATGVPDRFSGSGSGADFSLTISRL | |
| | | EPEDFAVYSCQQYGSSPLTFGPGTKVDVK | |
| FR1-A1 | VH | QVQLVQSGAEVKKPGSSVKVSCKASGQTFTGYYMHWVR | 5 |
| (ImClone/ | | QAPGQGLEWMGRIPILGIANYAQKFQGRVTITADKSTSTA | |
| Eli Lilly) | | YMELSSLRSEDTAVYYCARGGDLGGMDVWGQG | |
| | VL | EIVLTQSPLSLPVTPGEPASISCRSSQSLRHSNGYNYLDW | 6 |
| | | YLQKPGQSPQLLIYLASNRASGVPDRFSGSGSGTDFTLKI | |
| | | SRVEAEDVGVYYCMQALQIPPTFGPGTKVDK | |
| R1Mab1 | VH | EVQLVESGGGLVQPGGSLRLSCAASGFTFTSTWISWVPG | 7 |
| (Genentech) | | KGLEWVGEIDPYDGDTYYADSVKGRFTISADTSKNLQMN | |
| | | SLRAEDTAVYYCASSGYGGSDYAMDYWGQ | |
| | VL | DIQMTQSPSSLSASVGDRVTITCRASQDVSTAVAWYQQK | 8 |
| | | PGKAPKLLIYSASFLYSGVPSRFSGSGSGTDFTLTISSLQP | |
| | | EDFATYYCQQSYTTPPTFGQGTKWEIK | |
| R1Mab2 | VH | EVQLVESGGGLVQPGGSLRLSCAASGFTFSNNYIHWVPG | 9 |
| (Genentech) | | KGLEWVADIYPNDGDTDYADSVKGRFTISADTSKNLQMN | |
| | | SLRAEDTAVYYCAREHFDAWVHYYVMDYWGQ | |
| | VL | DIQMTQSPSSLSASVGDRVTITCRASQDVSTAVAWYQQK | 8 |
| | | PGKAPKLLIYSASFLYSGVPSRFSGSGSGTDFTLTISSLQP | |
| | | EDFATYYCQQSYTTPPTFGQGTKWEIK | |
| R1Mab3 | VH | EVQLVESGGGLVQPGGSLRLSCAASGFTFTSNWISWVPG | 10 |
| (Genentech) | | KGLEVWAEIDPYDGATDYADSVKGRFTISADTSKNLQMNS | |
| | | LRAEDTAVYYCATGTDWMDYWGQ | |
| | VL | DIQMTQSPSSLSASVGDRVTITCRASQDVSTAVAWYQQK | 8 |
| | | PGKAPKLLIYSASFLYSGVPSRFSGSGSGTDFTLTISSLQP | |
| | | EDFATYYCQQSYTTPPTFGQGTKWEIK | |

In further embodiments, GH1 domain binders and GH1 domain binder sequences that can be incorporated into the MBMs of the disclosure are identified in Tables 2A and 2B, respectively.

TABLE 2A
GH1 Domain Binders

| | | Sequence in Patent |
|---|---|---|
| Name | Reference | Reference |
| 39F7 (Amgen) | U.S. Pat. No. 8,263,074; | Heavy chain: Seq ID |
| | Min et al., 2018, J. Biol. | No. 82; Light chain: |
| | Chem. 293: 14678 | Seq ID No. 27 |

TABLE 2B
GH1 Domain Binders - VH and VL sequences

| Binder | Chain | Sequence |
|---|---|---|
| 39F7 | VH | QVQLVESGGGVVQPGRSLRLSCAASGFTFSNYGIH |
| (Amgen) | | WRQAPGKGLEWVAVIWYDGSIKYYADSVKGRFTIS |
| | | RDNSKNTLYLQMNSLRAEDTAVYYCARDRAAAGLH |
| | | YYYGMDVWGQGTTVTVSS (SEQ ID NO: 11) |
| | VL | EIVLTQSPGTLSLSPGERATLSCRASQSVSSTYLA |
| | | WYQQKPGQAPRLLIYGASSRATGIPDRFSGSGSGT |
| | | DFTLTISRLEPEDFAVYYCQQSGSSPLTFGGGTEV |
| | | EIK (SEQ ID NO: 12) |

In further embodiments, GH2 domain binders and GH2 domain binder sequences that can be incorporated into the MBMs of the disclosure are identified in Tables 3A and 3B, respectively.

TABLE 3A
GH2 Domain Binders

| | | Sequence in Patent |
|---|---|---|
| Name | Reference | Reference |
| 8C5.K4H3.M4L.KNV | US 2015/0218276 | Heavy chain: Seq ID |
| (Genentech) | | No. 129; Light chain: |
| | | Seq ID No. 131 |
| 5H23_humanized | U.S. Pat. No. 9,738,716 | Heavy chain: Seq ID |
| (NGM) | | No. 317; Light chain: |
| | | Seq ID No. 319 |

TABLE 3B
GH2 Domain Binders - VH and VL sequences

| Binder | Chain | Sequence |
|---|---|---|
| 5H23 | VH | QVQLQQSGAEVKKPGASVKVSCKASGYTFTSYDINW |
| (NGM) | (vH3) | VRQAPGQGLEWIGWIYPGDGSTKYNEKFKGKATITR |
| | | DTSASTAYMELSSLRSEDTAVYFCARSDYYGSRSFA |
| | | YWGQGTLVTVSS (SEQ ID NO: 13) |
| | VL | DIVMTQSPDSLAVSLGERATINCRASKSVSTSGYVY |
| | (vL2) | MHWYQQKPGQPPKLLIYLASYLESGVPDRFSGSGSG |
| | | TDFTLTISSVQAEDVAVYYCQHSRDLTFPFGGGTKL |
| | | EIK (SEQ ID NO: 14) |

Additional KLB binders are known in the art (e.g., mimAb1 (Amgen); see, e.g., US 2011/0135657 and Foltz et al., 2012, Sci. Transl. Med. 4:162ra153). The binding characteristics of KLB binders, e.g., whether they bind to an epitope in the GH1 domain or the GH2 domain can readily be ascertained by a skilled artisan using methods known in the art. Identifying the binding site of a KLB-binding antibody on KLB can be achieved via known techniques including, for example, array-based oligo-peptide scanning, cross-linking-coupled mass spectrometry, high-throughput shotgun mutagenesis epitope mapping, hydrogen-deuterium exchange, site-directed mutagenesis mapping, X-ray co-crystallography, and cryogenic electron microscopy. Alternatively, binding of a KLB binder to either the GH1 domain or the GH2 domain can be detected by, for example, an immunoassay such as an enzyme-linked immunosorbent assay (ELISA), Luminix bead-based assays, meso scale discovery (MSD), AlphaLISA, and flow cytometry.

Preferably, the binding to the GH1 and GH2 domains by the MBMs of the disclosure is non-competitive and non-blocking, i.e., the ABS that binds to the GH1 domain and the ABS that binds to the GH2 domain do not compete for binding to KLB. Assays for measuring binding competition between antibodies and antibody fragments are known in the art and include, for example, enzyme-linked immunosorbent assays (ELISA), fluorescence activated cell sorting (FACS) assays and surface plasmon resonance assays.

Competition for binding to a target molecule can be determined, for example, using a real time, label-free bio-layer interferometry assay on the Octet HTX biosensor platform (Pall ForteBio Corp.). In a specific embodiment of the assay, the entire assay is performed at 25° C. in a buffer of 10 mM HEPES, 150 mM NaCl, 3 mM EDTA, 1 mg/mL BSA, 0.05% v/v Surfactant Tween-20, pH 7.4 (HBS-EBT buffer) with the plate shaking at the speed of 1000 rpm. To assess whether two antibodies or antigen-binding fragments thereof are able to compete with one another for binding to their respective epitopes on their specific target antigen, a penta-His tagged (SEQ ID NO: 41) target antigen is first captured on to anti-penta-His (SEQ ID NO: 41) antibody coated Octet biosensor tips (Fortebio Inc, #18-5122) by submerging the biosensor tips in wells containing the penta-His tagged (SEQ ID NO: 41) target antigen. The antigen captured biosensor tips are then saturated with a first antibody or antigen-binding fragment thereof (subsequently referred to as Ab-1) by dipping into wells containing a solution of Ab-1 (e.g., a 50 μg/mL solution). The biosensor tips are then subsequently dipped into wells containing a solution (e.g., a 50 μg/mL solution) of a second antibody or antigen-binding fragment thereof (subsequently referred to as Ab-2). The biosensor tips are washed in HBS-EBT buffer in between every step of the assay. The real-time binding response can be monitored during the entire course of the assay and the binding response at the end of every step can be recorded. The response of Ab-2 binding to the target antigen pre-complexed with Ab-1 can be compared and competitive/non-competitive behavior of different antibodies/antigen-binding fragments against the same target antigen can be determined.

An MBM of the disclosure can thus include, for example, CDR or VH and/or VL sequences of any of the foregoing anti-FGFR1c or anti-KLB antibodies, for example any of the anti-FGFR1c, anti-GH1 domain or anti-GH2 domain antibodies provided in Tables 1A and 1B (for FGFR1c/ABS1), Tables 2A and 2B (for the KLB GH1 domain/ABS2), Tables 3A and 3B (for the KLB GH2 domain/ABS3), respectively.

The antigen binding sites of the MBMSs of the disclosure can be selected from immunoglobulin-based and non-immunoglobulin based binding domains.

In some embodiments, one or more of the ABSs are derived from an immunoglobulin, e.g., comprise or consist of a Fab (as described in Section 6.2.4), an scFv (as described in Section 6.2.3), or another an immunoglobulin-based format such as Fv, dsFv, (Fab′)2, a single domain antibody (SDAB), a VH or VL domain, or a camelid VHH domain (also called a nanobody).

An ABS can be derived from a single domain antibody composed of a single VH or VL domain which exhibits sufficient affinity to the target. In a specific embodiment, the single domain antibody is a camelid VHH domain (see, e.g., Riechmann, 1999, Journal of Immunological Methods 231:25-38; WO 94/04678).

In certain embodiments, one or more of the ABSs are derived from non-antibody scaffold proteins (including, but not limited to, designed ankyrin repeat proteins (DARPins), Avimers (short for avidity multimers), Anticalin/Lipocalins, Centyrins, Kunitz domains, Adnexins, Affilins, Affitins (also known as Nonfitins), Knottins, Pronectins, Versabodies, Duocalins, and Fynomers), ligands, receptors, cytokines or chemokines.

Non-immunoglobulin scaffolds that can be used in the MBMs of the disclosure include those listed in Tables 3 and 4 of Mintz and Crea, 2013, Bioprocess International 11(2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18. The contents of Tables 3 and 4 of Mintz and Crea, 2013, Bioprocess International 11 (2):40-48; in FIG. 1, Table 1 and Figure I of Vazquez-Lombardi et al., 2015, Drug Discovery Today 20(10):1271-83; in Table 1 and Box 2 of Skrlec et al., 2015, Trends in Biotechnology 33(7):408-18 (collectively, “Scaffold Disclosures”). In a particular embodiment, the Scaffold Disclosures are incorporated by reference for what they disclose relating to Adnexins. In another embodiment, the Scaffold Disclosures are incorporated by reference for what they disclose relating to Avimers. In another embodiment, the Scaffold Disclosures are incorporated by reference for what they disclose relating to Affibodies. In yet another embodiment, the Scaffold Disclosures are incorporated by reference for what they disclose relating to Anticalins. In yet another embodiment, the Scaffold Disclosures are incorporated by reference for what they disclose relating to DARPins. In yet another embodiment, the Scaffold Disclosures are incorporated by reference for what they disclose relating to Kunitz domains. In yet another embodiment, the Scaffold Disclosures are incorporated by reference for what they disclose relating to Knottins. In yet another embodiment, the Scaffold Disclosures are incorporated by reference for what they disclose relating to Pronectins. In yet another embodiment, the Scaffold Disclosures are incorporated by reference for what they disclose relating to Nanofitins. In yet another embodiment, the Scaffold Disclosures are incorporated by reference for what they disclose relating to Affilins. In yet another embodiment, the Scaffold Disclosures are incorporated by reference for what they disclose relating to Adnectins. In yet another embodiment, the Scaffold Disclosures are incorporated by reference for what they disclose relating to ABDs. In yet another embodiment, the Scaffold Disclosures are incorporated by reference for what they disclose relating to Adhirons. In yet another embodiment, the Scaffold Disclosures are incorporated by reference for what they disclose relating to Affimers. In yet another embodiment, the Scaffold Disclosures are incorporated by reference for what they disclose relating to Alphabodies. In yet another embodiment, the Scaffold Disclosures are incorporated by reference for what they disclose relating to Armadillo Repeat Proteins. In yet another embodiment, the Scaffold Disclosures are incorporated by reference for what they disclose relating to Atrimers/Tetranectins. In yet another embodiment, the Scaffold Disclosures are incorporated by reference for what they disclose relating to Obodies/OB-folds. In yet another embodiment, the Scaffold Disclosures are incorporated by reference for what they disclose relating to Centyrins. In yet another embodiment, the Scaffold Disclosures are incorporated by reference for what they disclose relating to Repebodies. In yet another embodiment, the Scaffold Disclosures are incorporated by reference for what they disclose relating to Anticalins. In yet another embodiment, the Scaffold Disclosures are incorporated by reference for what they disclose relating to Atrimers. In yet another embodiment, the Scaffold Disclosures are incorporated by reference for what they disclose relating to bicyclic peptides. In yet another embodiment, the Scaffold Disclosures are incorporated by reference for what they disclose relating to cys-knots. In yet another embodiment, the Scaffold Disclosures are incorporated by reference for what they disclose relating to Fn3 scaffolds (including Adnectins, Centryrins, Pronectins, and Tn3).

### 6.2.2. MBM Formats

In various aspects, an MBM of the disclosure comprises two half antibodies, one comprising two ABSs and the other comprising one ABS, the two halves paired through an Fc region.

In one aspect, the first half antibody comprises an scFv and an Fc domain, and the second half antibody comprises a Fab, an scFv and an Fc domain. The first and second half antibodies are associated through the Fc domains forming an Fc region. In various embodiments, the scFv domain in the second half antibody can be N-terminal to the Fab domain or C-terminal to the Fc domain.

In another aspect, the first half antibody comprises two Fab domains and an Fc domain, and the second half antibody comprises a Fab domain and an Fc domain. The first and second half antibodies are associated through the Fc domains forming an Fc region. In various embodiments, the second Fab domain in the first half antibody can be N-terminal to the first Fab domain (a configuration referred to as 2+1 N-Fab) or C-terminal to the Fc domain (a configuration referred to as 2+1 C-Fab).

In another aspect, the first half antibody comprises a Fab, an scFv and an Fc domain, and the second half antibody comprises a Fab domain and an Fc domain. The first and second half antibodies are associated through the Fc domains forming an Fc region. In various embodiments, the scFV domain in the first half antibody can be N-terminal to the Fab domain (a configuration referred to as 2+1 N-scFv) or C-terminal to the Fc domain (a configuration referred to as 2+1 C-scFv).

In another aspect, the first half antibody comprises an scFv and an Fc domain, and the second half antibody comprises two Fab domains and an Fc domain. The first and second half antibodies are associated through the Fc domains forming an Fc region. In various embodiments, the second Fab domain in the second half antibody can be N-terminal to the first Fab domain or C-terminal to the Fc domain.

In another aspect, the first half antibody comprises two Fab domains and an Fc domain, and the second half antibody comprises a non-immunoglobulin based ABS and an Fc domain. The first and second half antibodies are associated through the Fc domains forming an Fc region. In various embodiments, the second Fab domain in the first half antibody can be N-terminal to the first Fab domain or C-terminal to the Fc domain.

In another aspect, the first half antibody comprises a Fab, an scFv, and an Fc domain, and the second half antibody comprises a non-immunoglobulin based ABS and an Fc domain. The first and second half antibodies are associated through the Fc domains forming an Fc region. The scFV domain in the first half antibody can be N-terminal to the Fab domain or C-terminal to the Fc domain.

In a further aspect, the first half antibody comprises an scFv and an Fc domain, and the second half antibody comprises an scFv, an Fc domain, and a second scFv. The first and second half antibodies are associated through the Fc domains forming an Fc region. In various embodiments, the second scFv domain in the second half antibody can be N-terminal to the first scFv domain or C-terminal to the Fc domain.

Alternatively, the MBM can be a single chain. For example, the MBM can comprise three scFv domains connected through linkers.

In some embodiments, the MBM disclosure is or comprises antigen binding moieties arranged in the 2+1 N-scFv format. Accordingly, the disclosure provides an MBM comprising:
(a) a first polypeptide chain comprising, in an N- to C-terminal orientation, (i) an scFv operably linked to (ii) a first heavy chain region of a first Fab operably linked to (iii) an Fc region;
(b) a second polypeptide chain comprising, in an N- to C-terminal orientation, (i) a second heavy chain region of a second Fab operably linked to (ii) an Fc region;
(c) a third polypeptide chain comprising a first light chain that pairs with the first heavy chain region to form the first Fab;
(d) a fourth polypeptide chain comprising a second light chain that pairs with the second heavy chain region to form the second Fab.

The scFv can be in a VH-VL orientation or a VL-VH orientation.

In some embodiments, ABS1 is the first Fab, ABS2 is the scFv, and ABS3 is the second Fab.

In other embodiments, ABS1 is the first Fab, ABS3 is the scFv, and ABS2 is the second Fab.

In some embodiments, ABS2 is the first Fab, ABS1 is the scFv, and ABS3 is the second Fab.

In other embodiments, ABS2 is the first Fab, ABS3 is the scFv, and ABS1 is the second Fab.

In some embodiments, ABS3 is the first Fab, ABS2 is the scFv, and ABS1 is the second Fab.

In other embodiments, ABS3 is the first Fab, ABS1 is the scFv, and ABS2 is the second Fab.

The scFv can be linked to the first heavy chain region via a linker, e.g., a peptide linker of (a) at least 5 amino acids, at least 6 amino acids or at least 7 amino acids in length; and optionally (b) up to 30 amino acids, up to 40 amino acids, up to 50 amino acids or up to 60 amino acids in length. In various embodiments, the linker is 5 amino acids to 50 amino acids in length, 5 amino acids to 45 amino acids in length, 5 amino acids to 40 amino acids in length, 5 amino acids to 35 amino acids in length, 5 amino acids to 30 amino acids in length, 5 amino acids to 25 amino acids in length; 5 amino acids to 20 amino acids in length; 6 amino acids to 50 amino acids in length; 6 amino acids to 45 amino acids in length; 6 amino acids to 40 amino acids in length; 6 amino acids to 35 amino acids in length; 6 amino acids to 30 amino acids in length; 6 amino acids to 25 amino acids in length; 6 amino acids to 20 amino acids in length; 7 amino acids to 40 amino acids in length; 7 amino acids to 35 amino acids in length; 7 amino acids to 30 amino acids in length; 7 amino acids to 25 amino acids in length; 7 amino acids to 20 amino acids in length.

The peptide linker can comprise a multimer of GnS (SEQ ID NO: 15) or SGn (SEQ ID NO: 16), e.g., where n is an integer from 1 to 7 (e.g., a multimer of G4S (SEQ ID NO: 17)), and/or a multimer of glycines (e.g., two consecutive glycines (2Gly), three consecutive glycines (3Gly), four consecutive glycines (4Gly (SEQ ID NO: 18)), five consecutive glycines (5Gly (SEQ ID NO: 19)), six consecutive glycines (6Gly (SEQ ID NO: 20)), seven consecutive glycines (7Gly (SEQ ID NO: 21)), eight consecutive glycines (8Gly (SEQ ID NO: 22)) or nine consecutive glycines (9Gly (SEQ ID NO: 23))).

In some embodiments, the MBM disclosure is or comprises antigen binding moieties arranged in the 2+1 N-Fab format. Accordingly, the disclosure further provides an MBM comprising:
(a) a first polypeptide chain comprising, in an N- to C-terminal orientation, (i) a first heavy chain region of a first Fab operably linked to (ii) a second heavy chain region of a second Fab operably linked to (iii) an Fc region;
(b) a second polypeptide chain comprising, in an N- to C-terminal orientation, (i) a third heavy chain region of a third Fab operably linked to (ii) an Fc region;
(c) a third polypeptide chain comprising a first light chain that pairs with the first heavy chain region to form the first Fab;
(d) a fourth polypeptide chain comprising a second light chain that pairs with the second heavy chain region to form the second Fab; and
(e) a fifth polypeptide chain comprising a third light chain that pairs with the third heavy chain region to form the third Fab.

In some embodiments, ABS1 is the second Fab, ABS2 is the first Fab, and ABS3 is the third Fab.

In other embodiments, ABS1 is the second Fab, ABS3 is the first Fab, and ABS2 is the third Fab.

In some embodiments, ABS2 is the second Fab, ABS1 is the first Fab, and ABS3 is the third Fab.

In other embodiments, ABS2 is the second Fab, ABS3 is the first Fab, and ABS1 is the third Fab.

In some embodiments, ABS3 is the second Fab, ABS2 is the first Fab, and ABS1 is the third Fab.

The first and second Fabs, e.g., the first heavy chain region of the first Fab and the second heavy chain region of the second Fab, via a linker, e.g., a peptide linker of (a) at least 5 amino acids, at least 6 amino acids or at least 7 amino acids in length; and optionally (b) up to 30 amino acids, up to 40 amino acids, up to 45 amino acids, up to 50 amino acids or up to 60 amino acids in length. In various embodiments, the linker is 5 amino acids to 50 amino acids in length, 5 amino acids to 45 amino acids in length, 5 amino acids to 40 amino acids in length, 5 amino acids to 35 amino acids in length, 5 amino acids to 30 amino acids in length, 5 amino acids to 25 amino acids in length; 5 amino acids to 20 amino acids in length; 6 amino acids to 50 amino acids in length; 6 amino acids to 45 amino acids in length; 6 amino acids to 40 amino acids in length; 6 amino acids to 35 amino acids in length; 6 amino acids to 30 amino acids in length; 6 amino acids to 25 amino acids in length; 6 amino acids to 20 amino acids in length; 7 amino acids to 40 amino acids in length; 7 amino acids to 35 amino acids in length; 7 amino acids to 30 amino acids in length; 7 amino acids to 25 amino acids in length; 7 amino acids to 20 amino acids in length. The peptide linker can comprise a multimer of GnS (SEQ ID NO: 15) or SGn (SEQ ID NO: 16), e.g., where n is an integer from 1 to 7 (e.g., a multimer of G4S (SEQ ID NO: 17)), and/or a multimer of glycines (e.g., two consecutive glycines (2Gly), three consecutive glycines (3Gly), four consecutive glycines (4Gly (SEQ ID NO: 18)), five consecutive glycines (5Gly (SEQ ID NO: 19)), six consecutive glycines (6Gly (SEQ ID NO: 20)), seven consecutive glycines (7Gly (SEQ ID NO: 21)), eight consecutive glycines (8Gly (SEQ ID NO: 22)) or nine consecutive glycines (9Gly (SEQ ID NO: 23))).

In the foregoing embodiments, a Fab can be any Fab as described in Section 6.2.4 and an scFv can be any scFv as described in Section 6.2.3.

Preferably, the MBMs of the disclosure comprise an Fc heterodimer, for example as described in Section 6.2.7.2, and can also contain one or more mutations that reduce effector function, for example as described in Section 6.2.7.1.

Examples of Fc heterodimers include Fc regions with a star mutation and/or with knob-in-hole mutations. For example, in some embodiments, one Fc domain comprises a knob mutation and a second Fc domain comprises a hole mutation and a star mutation. In the 2+1 N-scFv and/or 2+1 C-scFv format, the Fc domain with the hole and star mutations can be on the scFv-containing chain or the non-scFv-containing chain. In other embodiments, one Fc domain comprises a knob mutation and star mutation and a second Fc domain comprises a hole mutation. In the 2+1 N-scFv and/or 2+1 C-scFv format, the Fc domain with the hole mutation can be on the scFv-containing chain or the non-scFv-containing chain. Similarly, in the 2+1 N-Fab and/or 2+1 C-Fab format, the Fc domain with the hole and star mutations can be on the half antibody comprising two Fab domains or the half antibody comprising a single Fab domain. In other embodiments, one Fc domain comprises a knob mutation and star mutation and a second Fc domain comprises a hole mutation. In the 2+1 N-Fab and/or 2+1 C-Fab format, the Fc domain with the hole mutation can be in the half antibody comprising two Fab domains or the half antibody comprising a single Fab domain.

In some embodiments, the MBMs of the disclosure have a pair of constant domains as set forth in Section 6.3 and/or as defined in specific embodiments 126 to 165.

### 6.2.3. scFv

Single chain Fv or “scFv” antibody fragments comprise the VH and VL domains of an antibody in a single polypeptide chain, are capable of being expressed as a single chain polypeptide, and retain the specificity of the intact antibodies from which they are derived. Generally, the scFv polypeptide further comprises a polypeptide linker between the VH and VL domain that enables the scFv to form the desired structure for target binding. Examples of linkers suitable for connecting the VH and VL chains of an scFV are the linkers identified in Section 6.2.5.

Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The scFv can comprise VH and VL sequences from any suitable species, such as murine, human or humanized VH and VL sequences.

To create an scFv-encoding nucleic acid, the VH and VL-encoding DNA fragments are operably linked to another fragment encoding a linker, e.g., encoding any of the linkers described in Section 6.2.5 (typically a repeat of a sequence containing the amino acids glycine and serine, such as the amino acid sequence (Gly4-Ser)₃ (SEQ ID NO: 24), such that the VH and VL sequences can be expressed as a contiguous single-chain protein, with the VL and VH regions joined by the flexible linker (see, e.g., Bird et al., 1988, Science 242:423-426; Huston et al., 1988, Proc. Natl. Acad. Sci. USA 85:5879-5883; McCafferty et al., 1990, Nature 348:552-554).

### 6.2.4. Fab

The MBMs of the disclosure can comprise one or more Fab domains and typically comprise at least one Fab domain in each half antibody. Fab domains were traditionally produced from by proteolytic cleavage of immunoglobulin molecules using enzymes such as papain. In the MBMs of the disclosure, the Fab domains are recombinantly expressed as part of a larger molecule.

The Fab domains can comprise constant domain and variable region sequences from any suitable species, and thus can be murine, chimeric, human or humanized.

Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

For the MBMs of the disclosure, particularly when the light chain is not a common or universal light chain, it is advantageous to use Fab heterodimerization strategies to permit the correct association of Fab domains belonging to the same ABS and minimize aberrant pairing of Fab domains belonging to different ABSs. For example, the Fab heterodimerization strategies shown in Table 4 below can be used:

TABLE 4
Fab Heterodimerization Strategies

| STRATEGY | VH | CH1 | VL | CL | REFERENCE |
|---|---|---|---|---|---|
| CrossMabCH1-CL | WT | CL domain | WT | CH1 domain | Schaefer et al., 2011, |
| | | | | | Cancer Cell 2011; 20: 472-86; |
| | | | | | PMID: 22014573. |
| orthogonal Fab | 39K, 62E | H172A, F174G | 1R, 38D, (36F) | L135Y, S176W | Lewis et al., 2014, Nat |
| VHVRD1CH1CRD2 - | | | | | Biotechnol 32: 191-8 |
| VLVRD1CλCRD2 | | | | | |
| orthogonal Fab | 39Y | WT | 38R | WT | Lewis et al., 2014, Nat |
| VHVRD2CH1wt - | | | | | Biotechnol 32: 191-8 |
| VLVRD2Cλwt | | | | | |
| TCR CαCβ | 39K | TCR Cα | 38D | TCR Cβ | Wu et al., 2015, MAbs 7: 364-76 |
| CR3 | WT | T192E | WT | N137K, S114A | Golay at al., 2016, J |
| | | | | | Immunol 196: 3199-211. |
| MUT4 | WT | L143Q, S188V | WT | V133T, S176V | Golay at al., 2016, J |
| | | | | | Immunol 196: 3199-211. |
| DuetMab | WT | F126C | WT | S121C | Mazor et al., 2015, MAbs |
| | | | | | 7: 377-89; Mazor et al., |
| | | | | | 2015, MAbs 7: 461-669. |
| Domain | WT | CH3 + knob or | WT | CH3 + hole or | Wozniak-Knopp et al., |
| exchanged | | hole mutation | | knob mutation | 2018, PLoSONE13(4): e0195442 |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or more amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179, the contents of which are hereby incorporated by reference.

In some embodiments, the Fab domain comprises a 192E substitution in the CH1 domain and 114A and 137K substitutions in the CL domain, which introduces a salt-bridge between the CH1 and CL domains (see, e.g., Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, e.g., Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1 R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121 C in the CL domain (see, e.g., Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the a T cell receptor and substituting the CL domain with the b domain of the T cell receptor, and pairing these domain replacements with an additional charge-charge interaction between the VL and VH domains by introducing a 38D modification in the VL domain and a 39K modification in the VH domain.

In lieu of, or in addition to, the use of Fab heterodimerization strategies to promote correct VH-VL pairings, the VL of common light chain (also referred to as a universal light chain) can be used for each Fab VL region of a MBM of the disclosure. In various embodiments, employing a common light chain as described herein reduces the number of inappropriate species of MBMs as compared to employing original cognate VLs. In various embodiments, the VL domains of the MBMs are identified from monospecific antibodies comprising a common light chain. In various embodiments, the VH regions of the MBMs comprise human heavy chain variable gene segments that are rearranged in vivo within mouse B cells that have been previously engineered to express a limited human light chain repertoire, or a single human light chain, cognate with human heavy chains and, in response to exposure with an antigen of interest, generate an antibody repertoire containing a plurality of human VHs that are cognate with one or one of two possible human VLs, wherein the antibody repertoire specific for the antigen of interest. Common light chains are those derived from a rearranged human VK1-39JK5 sequence or a rearranged human VK3-20JK1 sequence, and include somatically mutated (e.g., affinity matured) versions. See, for example, U.S. Pat. No. 10,412,940.

### 6.2.5. Linkers

In certain aspects, the present disclosure provides MBM in which two or more components of an ABS (e.g., a VH and a VL of an scFv), two or more ABSs (e.g., an scFv and a Fab of a half antibody), or an ABS and a non-ABS component (e.g., a Fab or scFv and an Fc domain) are connected to one another by a peptide linker. Such linkers are sometimes referred to herein an “ABS linkers.”

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker, e.g., a peptide linker separating an scFv and a heavy chain to its C-terminus, is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

Charged (e.g., charged hydrophilic linkers) and/or flexible linkers are particularly preferred.

Examples of flexible ABS linkers that can be used in the MBMs of the disclosure include those disclosed by Chen et al., 2013, Adv Drug Deliv Rev. 65(10): 1357-1369 and Klein et al., 2014, Protein Engineering, Design & Selection 27(10): 325-330. Particularly useful flexible linkers are or comprise repeats of glycines and serines, e.g., a monomer or multimer of GₙS (SEQ ID NO: 25) or SGₙ (SEQ ID NO: 26), where n is an integer from 1 to 10, e.g., 1 2, 3, 4, 5, 6, 7, 8, 9 or 10. In one embodiment, the linker is or comprises a monomer or multimer of repeat of G₄5 (SEQ ID NO: 17) e.g., (GGGGS)ₙ (SEQ ID NO: 17).

Polyglycine linkers can suitably be used in the MBMs of the disclosure. In some embodiments, the a peptide linker, e.g., a peptide linker separating an scFv domain and a heavy chain such as the scFv domain of ABS1 and the heavy chain variable region of ABS2, comprises two consecutive glycines (2Gly), three consecutive glycines (3Gly), four consecutive glycines (4Gly (SEQ ID NO: 18)), five consecutive glycines (5Gly (SEQ ID NO: 19)), six consecutive glycines (6Gly (SEQ ID NO: 20)), seven consecutive glycines (7Gly (SEQ ID NO: 21)), eight consecutive glycines (8Gly (SEQ ID NO: 22)) or nine consecutive glycines (9Gly (SEQ ID NO: 23)).

### 6.2.6. Hinge Regions

The MBMs of the disclosure can also comprise hinge regions, e.g., connecting an ABS module to an Fc region. The hinge region can be a native or a modified hinge region. Hinge regions are typically found at the N-termini of Fc regions.

A native hinge region is the hinge region that would normally be found between Fab and Fc domains in a naturally occurring antibody.

The term “hinge region”, unless the context dictates otherwise, refers to a naturally occurring (or native) or non-naturally occurring hinge sequence that in the context of a single or monomeric polypeptide chain is a monomeric hinge domain and in the context of a multimeric polypeptide (e.g., an MBM of the disclosure) comprising at least two separate polypeptide chains with hinge sequences that are associated. Sometimes, when describing the hinge sequence of a single polypeptide chain, the hinge region is referred to as a hinge “domain”. Typically, in a multimeric polypeptide comprising two associated hinge sequences, the two associated hinge sequences are identical.

A hinge region is composed of an upper hinge, a core hinge and a lower hinge.

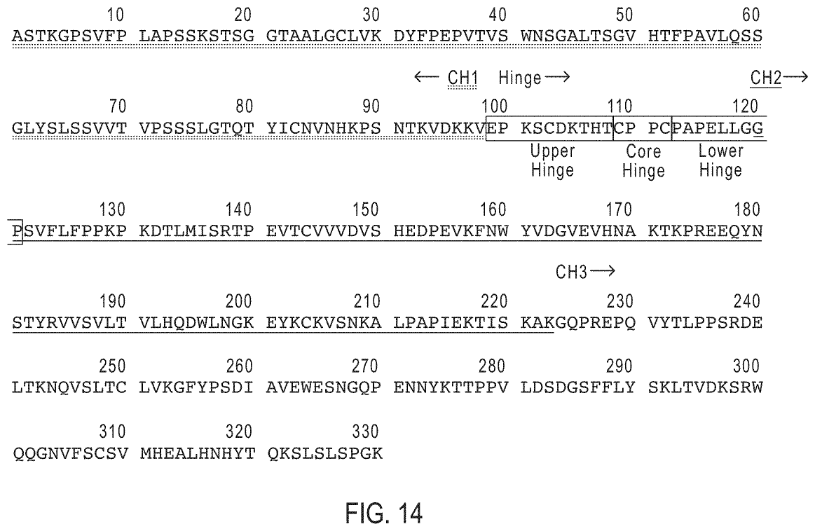

In human IgG1, the upper hinge corresponds to amino acids 99-108 of the sequence depicted in FIG. 14, the core hinge corresponds to amino acids 109-112 of the sequence depicted in FIG. 14, and the lower hinge corresponds to amino acids 113-121 of the sequence depicted in FIG. 14. The entire hinge sequence of human IgG1 is given as SEQ ID NO:68. As shown in FIG. 14, the last two amino acids of the lower hinge correspond to the first two amino acids of the CH2 domain.

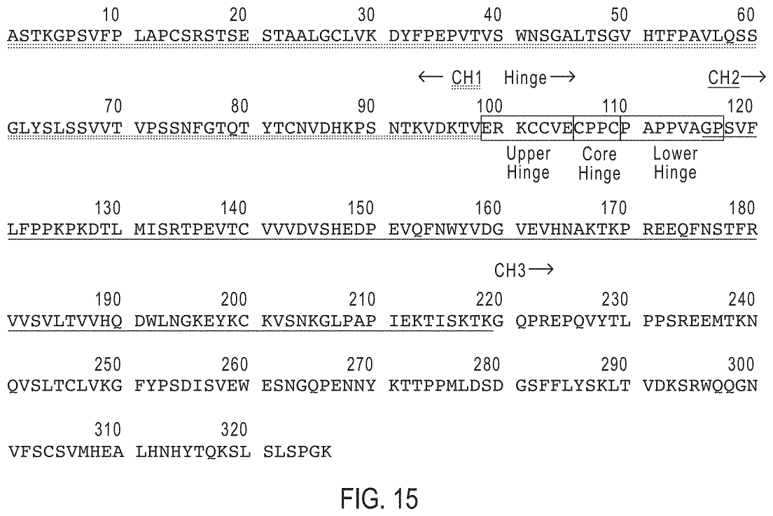

In human IgG2, the upper hinge corresponds to amino acids 99-105 of the sequence depicted in FIG. 15, the core hinge corresponds to amino acids 106-109 of the sequence depicted in FIG. 15, and the lower hinge corresponds to amino acids 110-117 of the sequence depicted in FIG. 15. The entire hinge sequence of human IgG1 is given as SEQ ID NO:69. As shown in FIG. 15, the last two amino acids of the lower hinge correspond to the first two amino acids of the CH2 domain.

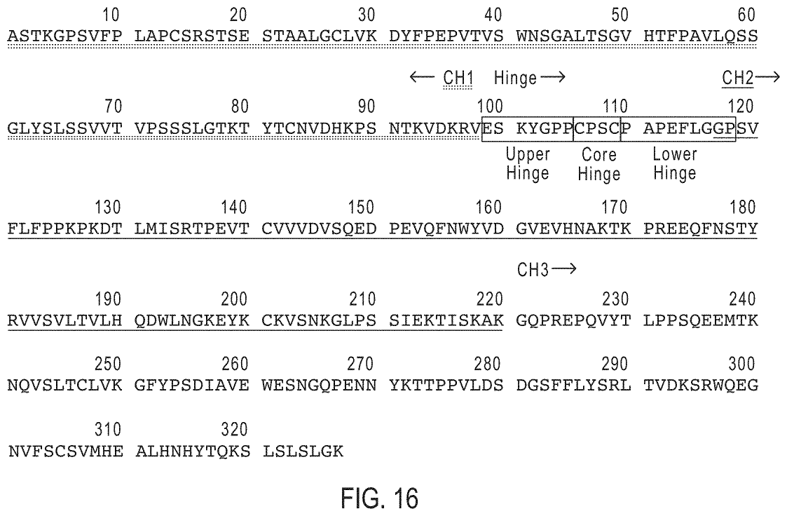

In human IgG4, the upper hinge corresponds to amino acids 99-105 of the sequence depicted in FIG. 16, the core hinge corresponds to amino acids 106-109 of the sequence depicted in FIG. 16, and the lower hinge corresponds to amino acids 110-118 of the sequence depicted in FIG. 16. The entire hinge sequence of human IgG4 is given as SEQ ID NO:72. As shown in FIG. 16, the last two amino acids of the lower hinge correspond to the first two amino acids of the CH2 domain.

A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions may comprise a complete hinge region derived from an antibody of a different class or subclass from that of the heavy chain Fc region. Alternatively, the modified hinge region may comprise part of a natural hinge or a repeating unit in which each unit in the repeat is derived from a natural hinge region. In a further alternative, the natural hinge region may be altered by converting one or more cysteine or other residues into neutral residues, such as serine or alanine, or by converting suitably placed residues into cysteine residues. By such means the number of cysteine residues in the hinge region may be increased or decreased. Other modified hinge regions may be entirely synthetic and may be designed to possess desired properties such as length, cysteine composition and flexibility.

A number of modified hinge regions have already been described for example, in U.S. Pat. No. 5,677,425, WO9915549, WO2005003170, WO2005003169, WO2005003170, WO9825971 and WO2005003171 and these are incorporated herein by reference.

In one embodiment, the Fc region of one or both half antibodies of the disclosure possesses an intact hinge region at its N-terminus.

In various embodiments, positions 233-236 within a hinge domain may be G, G, G and unoccupied; G, G, unoccupied, and unoccupied; G, unoccupied, unoccupied, and unoccupied; or all unoccupied, with positions numbered by EU numbering.

In some embodiments, the ABMs of the disclosure comprise a modified hinge domain that reduces binding affinity for an Fcγ receptor relative to a wild-type hinge domain of the same isotype (e.g., human IgG1 or human IgG4).

In one embodiment, the Fc region of one or both chains of the ABMs of disclosure possesses an intact hinge domain at its N-terminus. An Fc region comprising a hinge domain at its N-terminus is referred to herein as a “constant domain”. Exemplary constant domains are described herein and in Section 6.3.

In one embodiment both the Fc region and the hinge region of an ABM of the disclosure are derived from IgG4 and the hinge region comprises the modified sequence CPPC (SEQ ID NO: 27). The core hinge region of human IgG4 contains the sequence CPSC (SEQ ID NO: 28) compared to IgG1 that contains the sequence CPPC (SEQ ID NO: 27). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P (sometimes referred to as IgG4 S108P).

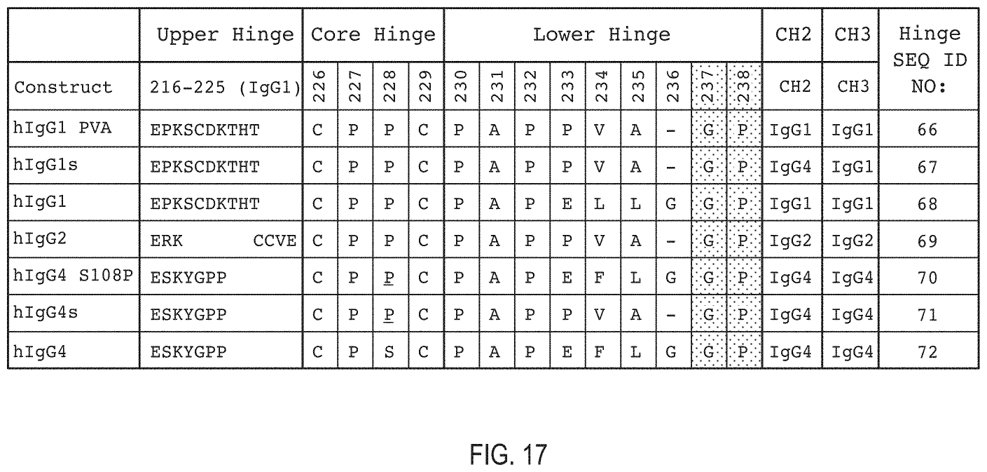

Exemplary hinge sequences which can be incorporated into the MBMs of the disclosure are set forth in FIG. 17, e.g., hinge sequences of any one of SEQ ID NOs. 66 to 72.

### 6.2.6.1. Chimeric Hinge Sequences

The hinge region can be a chimeric hinge region.

For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region.

In particular embodiments, a chimeric hinge region comprises the amino acid sequence EPKSCDKTHTCPPCPAPPVA (SEQ ID NO: 29) (previously disclosed as SEQ ID NO:8 of WO2014/121087, which is incorporated by reference in its entirety herein) or ESKYGPPCPPCPAPPVA (SEQ ID NO: 30) (previously disclosed as SEQ ID NO:9 of WO2014/121087). Such chimeric hinge sequences can be suitably linked to an IgG4 CH2 region (for example by incorporation into an IgG4 Fc domain, for example a human or murine Fc domain, which can be further modified in the CH2 and/or CH3 domain to reduce effector function, for example as described in Section 6.2.7.1).

Exemplary chimeric hinge sequences are set forth in FIG. 17 as SEQ ID NO:66, SEQ ID NO:67, SEQ ID NO:70, and SEQ ID NO:71.

### 6.2.6.2. Hinge Sequences with Reduced Effector Function

In further embodiments, the hinge region can be modified to reduce effector function, for example as described in WO2016161010A2, which is incorporated by reference in its entirety herein. In various embodiments, the positions 233-236 of the modified hinge region are G, G, G and unoccupied; G, G, unoccupied, and unoccupied; G, unoccupied, unoccupied, and unoccupied; or all unoccupied, with positions numbered by EU numbering (as shown in FIG. 1 of WO2016161010A2). These segments can be represented as GGG-, GG--, G--- or ---- with “-” representing an unoccupied position.

Position 236 is unoccupied in canonical human IgG2 but is occupied by in other canonical human IgG isotypes. Positions 233-235 are occupied by residues other than G in all four human isotypes (as shown in FIG. 1 of WO2016161010A2).

The hinge modification within positions 233-236 can be combined with position 228 being occupied by P. Position 228 is naturally occupied by P in human IgG1 and IgG2 but is occupied by S in human IgG4 and R in human IgG3. An S228P mutation in an IgG4 antibody is advantageous in stabilizing an IgG4 antibody and reducing exchange of heavy chain light chain pairs between exogenous and endogenous antibodies. Preferably positions 226-229 are occupied by C, P, P and C respectively.

Exemplary hinge regions have residues 226-236, sometimes referred to as middle (or core) and lower hinge, occupied by the modified hinge sequences designated GGG-(233-236), GG--(233-236), G---(233-236) and no G(233-236). Optionally, the hinge domain amino acid sequence comprises CPPCPAPGGG-GPSVF (SEQ ID NO: 31) (previously disclosed as SEQ ID NO:1 of WO2016161010A2), CPPCPAPGG--GPSVF (SEQ ID NO: 32) (previously disclosed as SEQ ID NO:2 of WO2016161010A2), CPPCPAPG---GPSVF (SEQ ID NO: 33) (previously disclosed as SEQ ID NO:3 of WO2016161010A2), or CPPCPAP----GPSVF (SEQ ID NO: 34) (previously disclosed as SEQ ID NO:4 of WO2016161010A2).

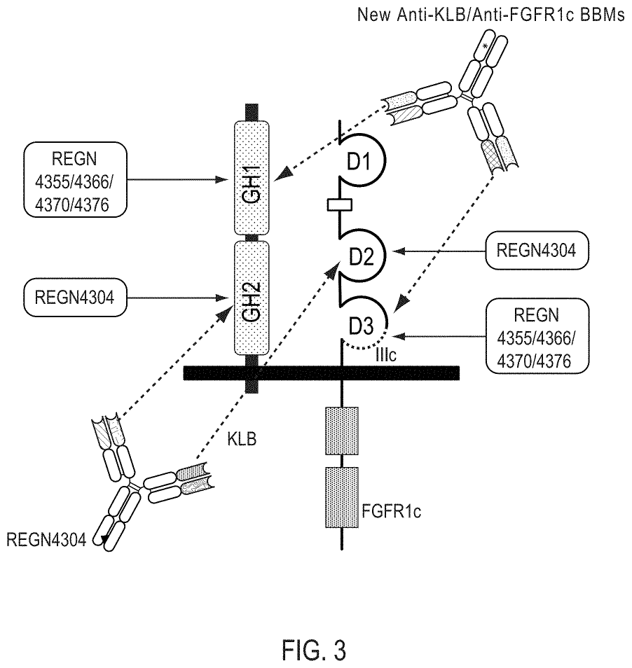

The modified hinge regions described above can be incorporated into a heavy chain constant region, which typically include CH2 and CH3 domains, and which may have an additional hinge segment (e.g., an upper hinge) flanking the designated region. Such additional constant region segments present are typically of the same isotype, preferably a human isotype, although can be hybrids of different isotypes. The isotype of such additional human constant regions segments is preferably human IgG4 but can also be human IgG1, IgG2, or IgG3 or hybrids thereof in which domains are of different isotypes. Exemplary sequences of human IgG1, IgG2 and IgG4 are shown in FIGS. 2-4 of WO2016161010A2.

In specific embodiments, the modified hinge sequences can be linked to an IgG4 CH2 region (for example by incorporation into an IgG4 Fc domain, for example a human or murine Fc domain, which can be further modified in the CH2 and/or CH3 domain to reduce effector function, for example as described in Section 6.2.7.1).

### 6.2.7. Fc Domains

The MBMs of the disclosure can include an Fc region derived from any suitable species. In one embodiment the Fc region is derived from a human Fc domain.

The Fc domain can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment the Fc domain is derived from IgG1. In one embodiment the Fc domain is derived from IgG4.

The two Fc domains within the Fc region can be the same or different from one another. In a native antibody the Fc domains are typically identical, but for the purpose of producing multispecific binding molecules, e.g., the MBMs of the disclosure, the Fc domains might advantageously be different to allow for heterodimerization, as described in Section 6.2.7.2 below.

In native antibodies, the heavy chain Fc domain of IgA, IgD and IgG is composed of two heavy chain constant domains (CH2 and CH3) and that of IgE and IgM is composed of three heavy chain constant domains (CH2, CH3 and CH4). These dimerize to create an Fc region.

In MBMs of the present disclosure, the Fc region, and/or the Fc domains within it, can comprise heavy chain constant domains from one or more different classes of antibody, for example one, two or three different classes.

In one embodiment the Fc region comprises CH2 and CH3 domains derived from IgG1.

In one embodiment the Fc region comprises CH2 and CH3 domains derived from IgG2.

In one embodiment the Fc region comprises CH2 and CH3 domains derived from IgG3.

In one embodiment the Fc region comprises CH2 and CH3 domains derived from IgG4.

In one embodiment the Fc region comprises a CH4 domain from IgM. The IgM CH4 domain is typically located at the C-terminus of the CH3 domain.

In one embodiment the Fc region comprises CH2 and CH3 domains derived from IgG and a CH4 domain derived from IgM.

It will be appreciated that the heavy chain constant domains for use in producing an Fc region for the MBMs of the present disclosure may include variants of the naturally occurring constant domains described above. Such variants may comprise one or more amino acid variations compared to wild type constant domains. In one example the Fc region of the present disclosure comprises at least one constant domain that varies in sequence from the wild type constant domain. It will be appreciated that the variant constant domains may be longer or shorter than the wild type constant domain. Preferably the variant constant domains are at least 60% identical or similar to a wild type constant domain. In another example the variant constant domains are at least 70% identical or similar. In another example the variant constant domains are at least 80% identical or similar. In another example the variant constant domains are at least 90% identical or similar. In another example the variant constant domains are at least 95% identical or similar.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the MBMs of the present disclosure do not comprise a tailpiece.

The Fc domains that are incorporated into the MBMs of the present disclosure may comprise one or more modifications that alter the functional properties of the proteins, for example, binding to Fc-receptors such as FcRn or leukocyte receptors, binding to complement, modified disulfide bond architecture, or altered glycosylation patterns.

Fc domains with modified disulfide bond architecture include CH3(S—S)-engineered Fc domains, e.g., by introduction of an E356C or a S354C mutation in one of the CH3 domains. Optionally, a Y349C mutation is introduced into the other CH3 domain (according to EU numbering).

Exemplary Fc modifications that alter effector function are described in Section 6.2.7.1.

The Fc domains can also be altered to include modifications that improve manufacturability of asymmetric MBMs, for example by allowing heterodimerization, which is the preferential pairing of non-identical Fc domains over identical Fc domains. Heterodimerization permits the production of MBMs in which different ABSs are connected to one another by an Fc region containing Fc domains that differ in sequence. Examples of heterodimerization strategies are exemplified in Section 6.2.7.2.

It will be appreciated that any of the modifications mentioned above can be combined in any suitable manner to achieve the desired functional properties and/or combined with other modifications to alter the properties of the MBMs.

### 6.2.7.1. Fc Domains with Altered Effector Function

In some embodiments, the Fc domain comprises one or more amino acid substitutions that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

In certain aspects, the Fc region with reduced effector function comprises an amino acid substitution at one or more of S228, E233, L234, L235, D265, N297, P329 and P331 (all according to EU numbering).

Exemplary substitutions at S228 include S228P.

Exemplary substitutions at E233 include E233A and E233P.

Exemplary substitutions at L234 include L234A.

Exemplary substitutions at L235 include L235A and L235E.

Exemplary substitutions at D265 include D265A.

Exemplary substitutions at N297 include N297A and N297D.

Exemplary substitutions at P329 include P329G or P329A.

Exemplary substitutions at P331 include P331S.

In some embodiments, the Fc region comprises an amino acid substitution at a position selected from the group of L234, L235 and P329 (numberings according to Kabat EU index). In some embodiments, the Fc region comprises the amino acid substitutions L234A and L235A (numberings according to Kabat EU index). In one such embodiment, the Fc region is an Igd Fc region, particularly a human Igd Fc region. In one embodiment, the Fc region comprises an amino acid substitution at position P329. In a more specific embodiment, the amino acid substitution is P329A or P329G, particularly P329G (numberings according to Kabat EU index). In one embodiment, the Fc region comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc region comprises amino acid substitutions at positions P329, L234 and L235 (numberings according to Kabat EU index). In more particular embodiments, the Fc region comprises the amino acid mutations L234A, L235A and P329G (“P329G LALA”, “PGLALA” or “LALAPG”).

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

Additional combinations of substitutions suitable for reducing effector function include (1) D265A/P329A, (2) D265A/N297A, (3) L234/L235A, and (4) P329A/L234A/L235A.

In one embodiment, the Fc domain is an IgG1 Fc domain, particularly a human IgG1 Fc domain.

Typically, the same one or more amino acid substitution is present in each of the two Fc domains of an Fc region. Thus, in a particular embodiment, each Fc domain of the Fc region comprises the amino acid substitutions L234A, L235A and P329G (Kabat EU index numbering), i.e. in each of the first and the second Fc domains in the Fc region the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index).

In one embodiment, the Fc domain is an IgG1 Fc domain, particularly a human IgG1 Fc domain. In some embodiments, the IgG1 Fc domain is a variant IgG1 comprising D265A, N297A mutations (EU numbering) to reduce effector function.

In another embodiment, the Fc domain is an IgG4 Fc domain with reduced binding to Fc receptors. Exemplary IgG4 Fc domains with reduced binding to Fc receptors may comprise an amino acid sequence selected from Table 5 below. In some embodiments, the Fc domain includes only the bolded portion of the sequences shown below:

TABLE 5

| Fc Domain | Sequence |
|---|---|
| SEQ ID NO: 1 of | Asp Lys Arg Val Glu Ser Lys Tyr Gly Pro Pro Cys Pro Pro Cys Pro |
| WO2014/121087 | Ala Pro Pro Val Ala Gly Pro Ser Val Phe Leu Phe Pro Pro Lys Pro |
| | Lys Asp Thr Leu Met Ile Ser Arg Thr Pro Glu Val Thr Cys Val Val |
| | Val Asp Val Ser Gln Glu Asp Pro Glu Val Gln Phe Asn Trp Tyr Val |
| | Asp Gly Val Glu Val His Asn Ala Lys Thr Lys Pro Arg Glu Glu Gln |
| | Phe Asn Ser Thr Tyr Arg Val Val Ser Val Leu Thr Val Leu His Gln |
| | Asp Trp Leu Asn Gly Lys Glu Tyr Lys Cys Lys Val Ser Asn Lys Gly |
| | Leu Pro Ser Ser Ile Glu Lys Thr Ile Ser Lys Ala Lys Gly Gln Pro Arg |
| | Glu Pro Gln Val Tyr Thr Leu Pro Pro Ser Gln Glu Glu Met Thr Lys |
| | Asn Gln Val Ser Leu Thr Cys Leu Val Lys Gly Phe Tyr Pro Ser Asp |
| | Ile Ala Val Glu Trp Glu Ser Asn Gly Gln Pro Glu Asn Asn Tyr Lys |
| | Thr Thr Pro Pro Val Leu Asp Ser Asp Gly Ser Phe Phe Leu Tyr Ser |
| | Arg Leu Thr Val Asp Lys Ser Arg Trp Gln Glu Gly Asn Val Phe Ser |
| | Cys Ser Val Met His Glu Ala Leu His Asn His Tyr Thr Gln Lys Ser |
| | Leu Ser Leu Ser Leu Gly Lys (SEQ ID NO: 35) |
| SEQ ID NO: 2 of | Asp Lys Lys Val Glu Pro Lys Ser Cys Asp Lys Thr His Thr Cys Pro |
| WO2014/121087 | Pro Cys Pro Ala Pro Pro Val Ala Gly Pro Ser Val Phe Leu Phe Pro |
| | Pro Lys Pro Lys Asp Thr Leu Met Ile Ser Arg Thr Pro Glu Val Thr |
| | Cys Val Val Val Asp Val Ser Gln Glu Asp Pro Glu Val Gln Phe Asn |
| | Trp Tyr Val Asp Gly Val Glu Val His Asn Ala Lys Thr Lys Pro Arg |
| | Glu Glu Gln Phe Asn Ser Thr Tyr Arg Val Val Ser Val Leu Thr Val |
| | Leu His Gln Asp Trp Leu Asn Gly Lys Glu Tyr Lys Cys Lys Val Ser |
| | Asn Lys Gly Leu Pro Ser Ser Ile Glu Lys Thr Ile Ser Lys Ala Lys Gly |
| | Gln Pro Arg Glu Pro Gln Val Tyr Thr Leu Pro Pro Ser Arg Asp Glu |
| | Leu Thr Lys Asn Gln Val Ser Leu Thr Cys Leu Val Lys Gly Phe Tyr |
| | Pro Ser Asp Ile Ala Val Glu Trp Glu Ser Asn Gly Gln Pro Glu Asn |
| | Asn Tyr Lys Thr Thr Pro Pro Val Leu Asp Ser Asp Gly Ser Phe Phe |
| | Leu Tyr Ser Lys Leu Thr Val Asp Lys Ser Arg Trp Gln Gln Gly Asn |
| | Val Phe Ser Cys Ser Val Met His Glu Ala Leu His Asn His Tyr Thr |
| | Gln Lys Ser Leu Ser Leu Ser Pro Gly Lys (SEQ ID NO: 36) |
| SEQ ID NO: 30 of | Ala Ser Thr Lys Gly Pro Ser Val Phe Pro Leu Ala Pro Ser Ser Lys |
| WO2014/121087 | Ser Thr Ser Gly Gly Thr Ala Ala Leu Gly Cys Leu Val Lys Asp Tyr |
| | Phe Pro Glu Pro Val Thr Val Ser Trp Asn Ser Gly Ala Leu Thr Ser |
| | Gly Val His Thr Phe Pro Ala Val Leu Gln Ser Ser Gly Leu Tyr Ser |
| | Leu Ser Ser Val Val Thr Val Pro Ser Ser Ser Leu Gly Thr Gln Thr |
| | Tyr Ile Cys Asn Val Asn His Lys Pro Ser Asn Thr Lys Val Asp Lys |
| | Lys Val Glu Pro Lys Ser Cys Asp Lys Thr His Thr Cys Pro Pro |
| | Cys Pro Ala Pro Pro Val Ala Gly Pro Ser Val Phe Leu Phe Pro |
| | Pro Lys Pro Lys Asp Thr Leu Met Ile Ser Arg Thr Pro Glu Val |
| | Thr Cys Val Val Val Asp Val Ser Gln Glu Asp Pro Glu Val Gln |
| | Phe Asn Trp Tyr Val Asp Gly Val Glu Val His Asn Ala Lys Thr |
| | Lys Pro Arg Glu Glu Gln Phe Asn Ser Thr Tyr Arg Val Val Ser |
| | Val Leu Thr Val Leu His Gln Asp Trp Leu Asn Gly Lys Glu Tyr |
| | Lys Cys Lys Val Ser Asn Lys Gly Leu Pro Ser Ser Ile Glu Lys |
| | Thr Ile Ser Lys Ala Lys Gly Gln Pro Arg Glu Pro Gln Val Tyr Thr |
| | Leu Pro Pro Ser Arg Asp Glu Leu Thr Lys Asn Gln Val Ser Leu |
| | Thr Cys Leu Val Lys Gly Phe Tyr Pro Ser Asp Ile Ala Val Glu |
| | Trp Glu Ser Asn Gly Gln Pro Glu Asn Asn |
| | Tyr Lys Thr Thr Pro Pro Val Leu Asp Ser Asp Gly Ser Phe Phe |
| | Leu Tyr Ser Lys Leu Thr Val Asp Lys Ser Arg Trp Gln Gln Gly |
| | Asn Val Phe Ser Cys Ser Val Met His Glu Ala Leu His Asn His |
| | Tyr Thr Gln Lys Ser Leu Ser Leu Ser Pro Gly Lys (SEQ ID NO: |
| | 37) |
| SEQ ID NO: 31 of | Ala Ser Thr Lys Gly Pro Ser Val Phe Pro Leu Ala Pro Cys Ser Arg |
| WO2014/121087 | Ser Thr Ser Glu Ser Thr Ala Ala Leu Gly Cys Leu Val Lys Asp Tyr |
| | Phe Pro Glu Pro Val Thr Val Ser Trp Asn Ser Gly Ala Leu Thr Ser |
| | Gly Val His Thr Phe Pro Ala Val Leu Gln Ser Ser Gly Leu Tyr Ser |
| | Leu Ser Ser Val Val Thr Val Pro Ser Ser Ser Leu Gly Thr Lys Thr |
| | Tyr Thr Cys Asn Val Asp His Lys Pro Ser Asn Thr Lys Val Asp Lys |
| | Arg Val Glu Ser Lys Tyr Gly Pro Pro Cys Pro Pro Cys Pro Ala |
| | Pro Pro Val Ala Gly Pro Ser Val Phe Leu Phe Pro Pro Lys Pro |
| | Lys Asp Thr Leu Met Ile Ser Arg Thr Pro Glu Val Thr Cys Val |
| | Val Val Asp Val Ser Gln Glu Asp Pro Glu Val Gln Phe Asn Trp |
| | Tyr Val Asp Gly Val Glu Val His Asn Ala Lys Thr Lys Pro Arg |
| | Glu Glu Gln Phe Asn Ser Thr Tyr Arg Val Val Ser Val Leu Thr |
| | Val Leu His Gln Asp Trp Leu Asn Gly Lys Glu Tyr Lys Cys Lys |
| | Val Ser Asn Lys Gly Leu Pro Ser Ser Ile Glu Lys Thr Ile Ser Lys |
| | Ala Lys Gly Gln Pro Arg Glu Pro Gln Val Tyr Thr Leu Pro Pro |
| | Ser Gln Glu Glu Met Thr Lys Asn |
| | Gln Val Ser Leu Thr Cys Leu Val Lys Gly Phe Tyr Pro Ser Asp |
| | Ile Ala Val Glu Trp Glu Ser Asn Gly Gln Pro Glu Asn Asn Tyr |
| | Lys Thr Thr Pro Pro Val Leu Asp Ser Asp Gly Ser Phe Phe Leu |
| | Tyr Ser Arg Leu Thr Val Asp Lys Ser Arg Trp Gln Glu Gly Asn |
| | Val Phe Ser Cys Ser Val Met His Glu Ala Leu His Asn His Tyr |
| | Thr Gln Lys Ser Leu Ser Leu Ser Leu Gly Lys (SEQ ID NO: 38) |
| SEQ ID NO: 37 of | Ala Ser Thr Lys Gly Pro Ser Val Phe Pro Leu Ala Pro Ser Ser Lys |
| WO2014/121087 | Ser Thr Ser Gly Gly Thr Ala Ala Leu Gly Cys Leu Val Lys Asp Tyr |
| | Phe Pro Glu Pro Val Thr Val Ser Trp Asn Ser Gly Ala Leu Thr Ser |
| | Gly Val His Thr Phe Pro Ala Val Leu Gln Ser Ser Gly Leu Tyr Ser |
| | Leu Ser Ser Val Val Thr Val Pro Ser Ser Ser Leu Gly Thr Gln Thr |
| | Tyr Ile Cys Asn Val Asn His Lys Pro Ser Asn Thr Lys Val Asp Lys |
| | Lys Val Glu Pro Lys Ser Cys Asp Lys Thr His Thr Cys Pro Pro |
| | Cys Pro Ala Pro Pro Val Ala Gly Pro Ser Val Phe Leu Phe Pro |
| | Pro Lys Pro Lys Asp Thr Leu Met Ile Ser Arg Thr Pro Glu Val |
| | Thr Cys Val Val Val Asp Val Ser Gln Glu Asp Pro Glu Val Gln |
| | Phe Asn Trp Tyr Val Asp Gly Val Glu Val His Asn Ala Lys Thr |
| | Lys Pro Arg Glu Glu Gln Phe Asn Ser Thr Tyr Arg Val Val Ser |
| | Val Leu Thr Val Leu His Gln Asp Trp Leu Asn Gly Lys Glu Tyr |
| | Lys Cys Lys Val Ser Asn Lys Gly Leu Pro Ser Ser Ile Glu Lys |
| | Thr Ile Ser Lys Ala Lys Gly Gln Pro Arg Glu Pro Gln Val Tyr Thr |
| | Leu Pro Pro Ser Arg Asp Glu Leu Thr Lys Asn Gln Val Ser Leu |
| | Thr Cys Leu Val Lys Gly Phe Tyr Pro Ser Asp Ile Ala Val Glu |
| | Trp Glu Ser Asn Gly Gln Pro Glu Asn Asn |
| | Tyr Lys Thr Thr Pro Pro Val Leu Asp Ser Asp Gly Ser Phe Phe |
| | Leu Tyr Ser Lys Leu Thr Val Asp Lys Ser Arg Trp Gln Gln Gly |
| | Asn Val Phe Ser Cys Ser Val Met His Glu Ala Leu His Asn Arg |
| | Phe Thr Gln Lys Ser Leu Ser Leu Ser Pro Gly Lys (SEQ ID NO: |
| | 39) |
| SEQ ID NO: 38 of | Ala Ser Thr Lys Gly Pro Ser Val Phe Pro Leu Ala Pro Cys Ser Arg |
| WO2014/121087 | Ser Thr Ser Glu Ser Thr Ala Ala Leu Gly Cys Leu Val Lys Asp Tyr |
| | Phe Pro Glu Pro Val Thr Val Ser Trp Asn Ser Gly Ala Leu Thr Ser |
| | Gly Val His Thr Phe Pro Ala Val Leu Gln Ser Ser Gly Leu Tyr Ser |
| | Leu Ser Ser Val Val Thr Val Pro Ser Ser Ser Leu Gly Thr Lys Thr |
| | Tyr Thr Cys Asn Val Asp His Lys Pro Ser Asn Thr Lys Val Asp Lys |
| | Arg Val Glu Ser Lys Tyr Gly Pro Pro Cys Pro Pro Cys Pro Ala |
| | Pro Pro Val Ala Gly Pro Ser Val Phe Leu Phe Pro Pro Lys Pro |
| | Lys Asp Thr Leu Met Ile Ser Arg Thr Pro Glu Val Thr Cys Val |
| | Val Val Asp Val Ser Gln Glu Asp Pro Glu Val Gln Phe Asn Trp |
| | Tyr Val Asp Gly Val Glu Val His Asn Ala Lys Thr Lys Pro Arg |
| | Glu Glu Gln Phe Asn Ser Thr Tyr Arg Val Val Ser Val Leu Thr |
| | Val Leu His Gln Asp Trp Leu Asn Gly Lys Glu Tyr Lys Cys Lys |
| | Val Ser Asn Lys Gly Leu Pro Ser Ser Ile Glu Lys Thr Ile Ser Lys |
| | Ala Lys Gly Gln Pro Arg Glu Pro Gln Val Tyr Thr Leu Pro Pro |
| | Ser Gln Glu Glu Met Thr Lys Asn |
| | Gln Val Ser Leu Thr Cys Leu Val Lys Gly Phe Tyr Pro Ser Asp |
| | Ile Ala Val Glu Trp Glu Ser Asn Gly Gln Pro Glu Asn Asn Tyr |
| | Lys Thr Thr Pro Pro Val Leu Asp Ser Asp Gly Ser Phe Phe Leu |
| | Tyr Ser Arg Leu Thr Val Asp Lys Ser Arg Trp Gln Glu Gly Asn |
| | Val Phe Ser Cys Ser Val Met His Glu Ala Leu His Asn Arg Phe |
| | Thr Gln Lys Ser Leu Ser Leu Ser Leu Gly Lys (SEQ ID NO: 40) |

In a particular embodiment, the IgG4 with reduced effector function comprises the bolded portion of the amino acid sequence of SEQ ID NO:31 of WO2014/121087, sometimes referred to herein as IgG4s or hIgG4s.

For heterodimeric ABMs, it is possible to incorporate a combination of the variant IgG4 Fc sequences set forth above, for example an Fc region comprising a combination of SEQ ID NO:30 of WO2014/121087 (or the bolded portion thereof) and SEQ ID NO:37 of WO2014/121087 (or the bolded portion thereof) or an Fc region comprising a combination of SEQ ID NO:31 of WO2014/121087 (or the bolded portion thereof) and SEQ ID NO:38 of WO2014/121087 (or the bolded portion thereof).

### 6.2.7.2. Fc Heterodimerization Variants

Many multispecific molecule formats entail dimerization between two Fc domains that, unlike a native immunoglobulin, are operably linked to non-identical antigen-binding domains (or portions thereof, e.g., a VH or VH-CH1 of a Fab). Inadequate heterodimerization of two Fc regions to form an Fc domain has can be an obstacle for increasing the yield of desired multispecific molecules and represents challenges for purification. A variety of approaches available in the art can be used in for enhancing dimerization of Fc domains that might be present in the MBMs of the disclosure, for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO2009/089004A1.

The present disclosure provides MBMs comprising Fc heterodimers, i.e., Fc regions comprising heterologous, non-identical Fc domains. Heterodimerization strategies are used to enhance dimerization of Fc regions operably linked to different ABSs (or portions thereof, e.g., a VH or VH-CH1 of a Fab) and reduce dimerization of Fc domains operably linked to identical ABSs. Typically, each Fc domain in the Fc heterodimer comprises a CH3 domain of an antibody. The CH3 domains are derived from the constant region of an antibody of any isotype, class or subclass, and preferably of IgG (IgG1, IgG2, IgG3 and IgG4) class, as described in the preceding section.

Heterodimerization of the two different heavy chains at CH3 domains give rise to the desired MBM, while homodimerization of identical heavy chains will reduce yield of the desired MBM. Thus, in a preferred embodiment, the two half antibodies that associate to form an MBM of the disclosure will contain CH3 domains with modifications that favor heterodimeric association relative to unmodified chains.

In a specific embodiment said modification promoting the formation of Fc heterodimers is a so-called “knob-into-hole” or “knob-in-hole” modification, comprising a “knob” modification in one of the Fc domains and a “hole” modification in the other Fc domain. The knob-into-hole technology is described, e.g., in U.S. Pat. Nos. 5,731,168; 7,695,936; Ridgway et al., 1996, Prot Eng 9:617-621, and Carter, 2001, Immunol Meth 248:7-15. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (′N). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis. An exemplary substitution is Y470T.

In a specific such embodiment, in the first Fc domain the threonine residue at position 366 is replaced with a tryptophan residue (T366W), and in the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second Fc domain additionally the tyrosine residue at position 349 is replaced by a cysteine residue (Y349C) (numbering according to Kabat EU index). In a particular embodiment, the first Fc domain comprises the amino acid substitutions S354C and T366W, and the second Fc domain comprises the amino acid substitutions Y349C, T366S, L368A and Y407V (numbering according to Kabat EU index).

In some embodiments, electrostatic steering (e.g., as described in Gunasekaran et al., 2010, J Biol Chem 285(25): 19637-46) can be used to promote the association of the first and the second subunit of the Fc domain.

As an alternative, or in addition, to the use of Fc domains that are modified to promote heterodimerization, an Fc domain can be modified to allow a purification strategy that enables selections of Fc heterodimers. In one such embodiment, one half antibody comprises a modified Fc domain that abrogates its binding to Protein A, thus enabling a purification method that yields a heterodimeric protein. See, for example, U.S. Pat. No. 8,586,713. As such, the MBMs comprise a first CH3 domain and a second Ig CH3 domain, wherein the first and second Ig CH3 domains differ from one another by at least one amino acid, and wherein at least one amino acid difference reduces binding of the MBM to Protein A as compared to a corresponding MBM lacking the amino acid difference. In one embodiment, the first CH3 domain binds Protein A and the second CH3 domain contains a mutation/modification that reduces or abolishes Protein A binding such as an H95R modification (by IMGT exon numbering; H435R by EU numbering). The second CH3 may further comprise a Y96F modification (by IMGT; Y436F by EU). Thus class of modifications is referred to herein as “star” mutations.

In certain aspects, the MBMs of the disclosure can include both knob-in-hole mutations and star mutations to facilitate purification. In various embodiments, one half antibody contains a knob or hole mutation and the other half antibody comprises the corresponding hole or knob mutations. Thus, in some embodiments, the Fc domain of one half antibody comprises one or more knob mutations and a star mutation, and the Fc domain of the other half antibody comprises one or more hole mutations. In other embodiments, the Fc domain of one half antibody comprises one or more hole mutations and a star mutation, and the Fc domain of the other half antibody comprises one or more knob mutation.

### 6.3. Constant Domains

The MBMs of the disclosure generally comprise two half antibodies. Typically, each half antibody comprises a constant domain composed of a CH2 and CH3 domain (e.g., as described in the context of the Fc domains of Section 6.2.7) with a hinge domain (e.g., as described in Section 6.2.6) at its N-terminus. Each constant domain may be fused at its N-terminus an antigen binding site or one of its polypeptide chains, for example the CH1 portion of a Fab domain.

In some embodiments, the constant domain has any of the configurations or sequences set forth in FIG. 17. In various embodiments, the constant domain comprising a hinge having of the hinge sequences set forth in FIG. 17 (e.g., any one of SEQ ID NOS:66 to 72) with wild type or modified CH2 and/or CH3 domains, e.g., modified to modified to reduce effector function, facilitate correct heterodimer formation, modified to facilitate purification, etc. Exemplary modifications are set forth in Section 6.2.7, including its subsections Section 6.2.7.1 and Section 6.2.7.2.

In some embodiments, an MBM of the disclosure comprises a constant domain comprising an amino acid sequence according to the amino acid sequence of SEQ ID NO:45, a constant domain comprising an amino acid sequence according to the amino acid sequence of SEQ ID NO:46, a constant domain comprising an amino acid sequence according to the amino acid sequence of SEQ ID NO:48, a constant domain comprising an amino acid sequence according to the amino acid sequence of SEQ ID NO:49, a constant domain comprising an amino acid sequence according to the amino acid sequence of SEQ ID NO:50, a constant domain comprising an amino acid sequence according to the amino acid sequence of SEQ ID NO:51, a constant domain comprising an amino acid sequence according to the amino acid sequence of SEQ ID NO:52, a constant domain comprising an amino acid sequence according to the amino acid sequence of SEQ ID NO:53, a constant domain comprising an amino acid sequence according to the amino acid sequence of SEQ ID NO:54, a constant domain comprising an amino acid sequence according to the amino acid sequence of SEQ ID NO:58, a constant domain comprising an amino acid sequence according to the amino acid sequence of SEQ ID NO:59, a constant domain comprising an amino acid sequence according to the amino acid sequence of SEQ ID NO:60, a constant domain comprising an amino acid sequence according to the amino acid sequence of SEQ ID NO:61, a constant domain comprising an amino acid sequence according to the amino acid sequence of SEQ ID NO:62, a constant domain comprising an amino acid sequence according to the amino acid sequence of SEQ ID NO:63, a constant domain comprising an amino acid sequence according to the amino acid sequence of SEQ ID NO:64, a constant domain comprising an amino acid sequence according to the amino acid sequence of SEQ ID NO:65, or a constant comprising an amino acid sequence having at least 90%, at least 93%, at least 95%, at least 96%, at least 97%, at least 98%, at least 99% or 100% sequence identity to the amino acid sequence provided by any of the foregoing sequence identifiers.

In some embodiments, the constant domains are “chimeric”, comprising constant domain sequences from more than one immunoglobulin isotype. In some embodiments, the chimeric constant domains have sequence from different IgG isotypes (e.g., any two of IgG1, IgG2, IgG3, and IgG4).

An exemplary chimeric constant domain is what is referred to herein as an “IgG1 PVA” isotype or similar terms, comprising an IgG1 upper hinge domain, an IgG1 core hinge domain, and an IgG1 lower hinge domain having a substitution/deletion mutation ELLG→PVA- (or “P-V-A-absent”) (“ELLG” disclosed as SEQ ID NO: 79) at amino acid positions 233-236 (EU numbering), an IgG1 CH2 domain, and an IgG1 CH3 domain. The ELLG→PVA- (or “P-V-A-absent”) (“ELLG” disclosed as SEQ ID NO: 79) modifications incorporate IgG2 sequences into IgG1. In certain aspects, the chimeric constant domain comprises an amino acid sequence having at least 90%, at least 93%, at least 95%, at least 96%, at least 97%, at least 98% sequence identity to SEQ ID NO:46 (hIgG1 PVA constant domain).

Chimeric constant domain can be further modified to, e.g., further alter effector function (e.g., as described in Section 6.2.7.1) and/or facilitate correct pairing or purification of MBMs with asymmetrical half antibodies (e.g., as described in Section 6.2.7.2).

In particular embodiments, an MBM of the disclosure two constant domains comprising an Fc heterodimer, wherein the two constant domains comprise an amino acid sequence having at least 90%, at least 93%, at least 95%, at least 96%, at least 97%, or at least 98% sequence identity to SEQ ID NO:46 (hIgG1 PVA constant domain), wherein:
a) both constant domains comprise the P-V-A-absent sequence at amino acid positions 233-236 (EU numbering);
b) one constant domain comprises knob mutation T366W and the other constant domain comprises hole mutations T366S, L368A and Y407V;
c) optionally, one or both constant domains comprise star mutations H435R and Y436F; and
d) both or neither constant domains comprise disulfide architecture mutation S354C or E356C.

In a particular embodiment, an MBM of the disclosure comprises two constant domains comprising an Fc heterodimer, wherein the two constant domains comprise:
a) a first constant domain comprising an amino acid sequence having at least 90%, at least 93%, at least 95%, at least 96%, at least 97%, at least 98%, at least 99% or 100% sequence identity to SEQ ID NO:58, provided that if the amino acid sequence has less than 100% identity to SEQ ID NO:58 the sequence retains the PVA modification (P-V-A-absent at amino acid positions 233-236 (EU numbering)) in the hinge and the knob mutation T366W; and
b) a second constant domain comprising an amino acid sequence having at least 90%, at least 93%, at least 95%, at least 96%, at least 97%, at least 98%, at least 99% or 100% sequence identity to SEQ ID NO: 62, provided that if the amino acid sequence has less than 100% identity to SEQ ID NO:62 the sequence retains the PVA modification (P-V-A-absent at amino acid positions 233-236 (EU numbering)) in the hinge and the hole mutations T366S, L368A and Y407V.

In another particular embodiment, an MBM of the disclosure two constant domains comprising an Fc heterodimer, wherein the two constant domains comprise:
a) a first constant domain comprising an amino acid sequence having at least 90%, at least 93%, at least 95%, at least 96%, at least 97%, at least 98%, at least 99% or 100% sequence identity to SEQ ID NO:58, provided that if the amino acid sequence has less than 100% identity to SEQ ID NO:58 the sequence retains the PVA modification (P-V-A-absent at amino acid positions 233-236 (EU numbering)) in the hinge and the knob mutation T366W; and
b) a second constant domain comprising an amino acid sequence having at least 90%, at least 93%, at least 95%, at least 96%, at least 97%, at least 98%, at least 99% or 100% sequence identity to SEQ ID NO: 63, provided that if the amino acid sequence has less than 100% identity to SEQ ID NO:63 the sequence retains the PVA modification (P-V-A-absent at amino acid positions 233-236 (EU numbering)) in the hinge, the hole mutations T366S, L368A and Y407V, and the star mutations H435R and Y436F.

In another particular embodiment, an MBM of the disclosure two constant domains comprising an Fc heterodimer, wherein the two constant domains comprise:
a) a first constant domain comprising an amino acid sequence having at least 90%, at least 93%, at least 95%, at least 96%, at least 97%, at least 98%, at least 99% or 100% sequence identity to SEQ ID NO:59, provided that if the amino acid sequence has less than 100% identity to SEQ ID NO:59 the sequence retains the PVA modification (P-V-A-absent at amino acid positions 233-236 (EU numbering)) in the hinge, the knob mutation T366W, and the star mutations H435R and Y436F; and
b) a second constant domain comprising an amino acid sequence having at least 90%, at least 93%, at least 95%, at least 96%, at least 97%, at least 98%, at least 99% or 100% sequence identity to SEQ ID NO: 62, provided that if the amino acid sequence has less than 100% identity to SEQ ID NO:62 the sequence retains the PVA modification (P-V-A-absent at amino acid positions 233-236 (EU numbering)) in the hinge and the hole mutations T366S, L368A and Y407V.

In another particular embodiment, an MBM of the disclosure two constant domains comprising an Fc heterodimer, wherein the two constant domains comprise:
a) a first constant domain comprising an amino acid sequence having at least 90%, at least 93%, at least 95%, at least 96%, at least 97%, at least 98%, at least 99% or 100% sequence identity to SEQ ID NO:59, provided that if the amino acid sequence has less than 100% identity to SEQ ID NO:59 the sequence retains the PVA modification (P-V-A-absent at amino acid positions 233-236 (EU numbering)) in the hinge, the knob mutation T366W, and the star mutations H435R and Y436F; and
b) a second constant domain comprising an amino acid sequence having at least 90%, at least 93%, at least 95%, at least 96%, at least 97%, at least 98%, at least 99% or 100% sequence identity to SEQ ID NO: 63, provided that if the amino acid sequence has less than 100% identity to SEQ ID NO:63 the sequence retains the PVA modification (P-V-A-absent at amino acid positions 233-236 (EU numbering)) in the hinge, the hole mutations T366S, L368A and Y407V, and the star mutations H435R and Y436F.

In another particular embodiment, an MBM of the disclosure comprises two constant domains comprising an Fc heterodimer, wherein the two constant domains comprise:
a) a first constant domain comprising an amino acid sequence having at least 90%, at least 93%, at least 95%, at least 96%, at least 97%, at least 98%, at least 99% or 100% sequence identity to SEQ ID NO:60, provided that if the amino acid sequence has less than 100% identity to SEQ ID NO:60 the sequence retains the PVA modification (P-V-A-absent at amino acid positions 233-236 (EU numbering)) in the hinge, the disulfide architecture mutation S354C, and the knob mutation T366W; and
b) a second constant domain comprising an amino acid sequence having at least 90%, at least 93%, at least 95%, at least 96%, at least 97%, at least 98%, at least 99% or 100% sequence identity to SEQ ID NO: 64, provided that if the amino acid sequence has less than 100% identity to SEQ ID NO:64 the sequence retains the PVA modification (P-V-A-absent at amino acid positions 233-236 (EU numbering)) in the hinge, the disulfide architecture mutation S354C, and the hole mutations T366S, L368A and Y407V.

In another particular embodiment, an MBM of the disclosure two constant domains comprising an Fc heterodimer, wherein the two constant domains comprise:
a) a first constant domain comprising an amino acid sequence having at least 90%, at least 93%, at least 95%, at least 96%, at least 97%, at least 98%, at least 99% or 100% sequence identity to SEQ ID NO:60, provided that if the amino acid sequence has less than 100% identity to SEQ ID NO:60 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), and the knob mutation T366W; and
b) a second constant domain comprising an amino acid sequence having at least 90%, at least 93%, at least 95%, at least 96%, at least 97%, at least 98%, at least 99% or 100% sequence identity to SEQ ID NO: 65, provided that if the amino acid sequence has less than 100% identity to SEQ ID NO:65 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the hole mutations T366S, L368A and Y407V, and the star mutations H435R and Y436F.

In another particular embodiment, an MBM of the disclosure two constant domains comprising an Fc heterodimer, wherein the two constant domains comprise:
a) a first constant domain comprising an amino acid sequence having at least 90%, at least 93%, at least 95%, at least 96%, at least 97%, at least 98%, at least 99% or 100% sequence identity to SEQ ID NO:61, provided that if the amino acid sequence has less than 100% identity to SEQ ID NO:61 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the knob mutation T366W, and the star mutations H435R and Y436F; and
b) a second constant domain comprising an amino acid sequence having at least 90%, at least 93%, at least 95%, at least 96%, at least 97%, at least 98%, at least 99% or 100% sequence identity to SEQ ID NO: 64, provided that if the amino acid sequence has less than 100% identity to SEQ ID NO:64 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), and the hole mutations T366S, L368A and Y407V.

In another particular embodiment, an MBM of the disclosure two constant domains comprising an Fc heterodimer, wherein the two constant domains comprise:
a) a first constant domain comprising an amino acid sequence having at least 90%, at least 93%, at least 95%, at least 96%, at least 97%, at least 98%, at least 99% or 100% sequence identity to SEQ ID NO:61, provided that if the amino acid sequence has less than 100% identity to SEQ ID NO:61 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the knob mutation T366W, and the star mutations H435R and Y436F; and
b) a second constant domain comprising an amino acid sequence having at least 90%, at least 93%, at least 95%, at least 96%, at least 97%, at least 98%, at least 99% or 100% sequence identity to SEQ ID NO: 65, provided that if the amino acid sequence has less than 100% identity to SEQ ID NO:65 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the hole mutations T366S, L368A and Y407V, and the star mutations H435R and Y436F.

In yet further embodiments, an MBM of the disclosure two constant domains comprising an Fc heterodimer, wherein the two constant domains comprise an amino acid sequence having at least 90%, at least 93%, at least 95%, at least 96%, at least 97%, or at least 98% sequence identity to SEQ ID NO:49 (hIgG1 N180G, also referred to as hIgG1 N297G), wherein:
a) both constant domains comprise the N180G/N297G amino acid substitution;
b) one constant domain comprises knob mutation T366W and the other constant domain comprises hole mutations T366S, L368A and Y407V;
c) optionally, one or both constant domains comprise star mutations H435R and Y436F; and
d) both or neither constant domains comprise disulfide architecture mutation S354C or E356C.

In yet further embodiments, an MBM of the disclosure two constant domains comprising an Fc heterodimer, wherein the two constant domains comprise an amino acid sequence having at least 90%, at least 93%, at least 95%, at least 96%, at least 97%, or at least 98% sequence identity to SEQ ID NO:53 (hIgG4 S108P, also referred to as hIgG4 S228P), wherein:
a) both constant domains comprise the S108P/S228P amino acid substitution;
b) one constant domain comprises knob mutation T366W and the other constant domain comprises hole mutations T366S, L368A and Y407V;
c) optionally, one or both constant domains comprise star mutations H435R and Y436F; and
d) both or neither constant domains comprise disulfide architecture mutation S354C or E356C.

In yet further embodiments, an MBM of the disclosure two constant domains comprising an Fc heterodimer, wherein the two constant domains comprise an amino acid sequence having at least 90%, at least 93%, at least 95%, at least 96%, at least 97%, or at least 98% sequence identity to SEQ ID NO:54 (variant IgG4 with S108P, also referred to as hIgG4 S228P, substitution and IgG1 CH2 and CH3 domains), wherein:
a) both constant domains comprise the S108P/S228P amino acid substitution;
b) one constant domain comprises knob mutation T366W and the other constant domain comprises hole mutations T366S, L368A and Y407V;
c) optionally, one or both constant domains comprise star mutations H435R and Y436F; and
d) both or neither constant domains comprise disulfide architecture mutation S354C or E356C.

### 6.4. Nucleic Acids and Host Cells

In another aspect, the disclosure provides nucleic acids encoding the MBMs of the disclosure. In some embodiments, the MBMs are encoded by a single nucleic acid. In other embodiments, the MBMs are encoded by a plurality (e.g., two, three, four or more) nucleic acids.

A single nucleic acid can encode a MBM that comprises a single polypeptide chain, a MBM that comprises two or more polypeptide chains, or a portion of a MBM that comprises more than two polypeptide chains (for example, a single nucleic acid can encode two polypeptide chains of a MBM comprising three, four or more polypeptide chains, or three polypeptide chains of a MBM comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a MBM comprising two or more polypeptide chains is encoded by two or more nucleic acids. The number of nucleic acids encoding a MBM can be equal to or less than the number of polypeptide chains in the MBM (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.4.1. Vectors

The disclosure provides vectors comprising nucleotide sequences encoding a MBM or a MBM component described herein, for example one or two of the polypeptide chains of a half antibody. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. Methods and conditions for culturing the resulting transfected cells and for recovering the expressed polypeptides are known to those skilled in the art, and may be varied or optimized depending upon the specific expression vector and mammalian host cell employed, based upon the present description.

### 6.4.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a mammalian, e.g., human, cell. Suitable eukaryotic cells include, but are not limited to, Vero cells, HeLa cells, COS cells, CHO cells, HEK293 cells, BHK cells and MDCKII cells. Derivatives of the foregoing cell types are also included (such as, but not limited to, Expi293, a derivative of HEK293 which has been adapted for higher density growth). Suitable insect cells include, but are not limited to, Sf9 cells.

### 6.5. Pharmaceutical Compositions

The MBMs of the disclosure may be in the form of compositions comprising the MBM and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the MBM and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intrathecally, topically or locally. The most suitable route for administration in any given case will depend on the particular subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an MBM of the disclosure per dose. The quantity of MBM included in a unit dose will depend on the disease being treated, as well as other factors as are well known in the art. Such unit dosages may be in the form of a lyophilized dry powder containing an amount of MBM suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of MBM suitable for a single administration.

The pharmaceutical compositions may also be supplied in bulk form containing quantities of MBM suitable for multiple administrations.

Pharmaceutical compositions may be prepared for storage as lyophilized formulations or aqueous solutions by mixing an MBM having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations, but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth, and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of MBM.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188 etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

### 6.6. Therapeutic Indications

The MBMs and pharmaceutical compositions of the disclosure can be used for treating a metabolic condition and/or improving metabolism in a subject. MBMs and pharmaceutical compositions of the disclosure are useful in the treatment of any disease or condition that may be improved or ameliorated by stimulating, mimicking, and/or promoting FGF21 signaling. This is generally achieved by the MBMs of the disclosure by agonizing (i.e., stimulating) the FGF21 receptor complex. MBMs and pharmaceutical compositions of the disclosure can be used for the treatment or prevention of any disease or condition that may be improved by lowering blood glucose levels, activating glucose uptake in a subject, or increasing insulin sensitivity.

In some embodiments, the MBMs and pharmaceutical compositions of the disclosure can be used for treating nonalcoholic steatohepatitis (“NASH”), treating nonalcoholic fatty liver disease (NAFLD), treating metabolic disease, reducing circulating HDL cholesterol, increasing circulating LDL cholesterol, reducing blood triglycerides, reducing blood glucose, treating obesity, and treating diabetes.

Thus, in one aspect, the disclosure provides a method of reducing circulating HDL cholesterol comprising administering to a subject suffering from elevated HDL levels an effective amount of an MBM or pharmaceutical composition of the disclosure.

In another aspect, the disclosure provides a method of increasing circulating LDL cholesterol comprising administering to a subject suffering from low LDL levels an effective amount of an MBM or pharmaceutical composition of the disclosure.

In another aspect, the disclosure provides a method of reducing blood triglycerides comprising administering to a subject suffering from elevated triglyceride levels an effective amount of an MBM or pharmaceutical composition of the disclosure.

In another aspect, the disclosure provides a method of reducing blood glucose comprising administering to a subject suffering from suffering from elevated glucose levels an effective amount of an MBM or pharmaceutical composition of the disclosure.

In another aspect, the disclosure provides a method of treating obesity comprising administering to a subject suffering from obesity an effective amount of an MBM or pharmaceutical composition of the disclosure.

In another aspect, the disclosure provides a method of treating diabetes comprising administering to a subject suffering from diabetes an effective amount of an MBM or pharmaceutical composition of the disclosure.

### 7. EXAMPLES

### 7.1. Example 1: Constructs of the Disclosure

### 7.1.1. Antibodies that Bind to KLB and FGFR1c

Antibody screening campaigns were performed to identify antibodies that bind to human KLB and antibodies that bind to human FGFR1c. The following antibodies were identified:
Antibodies that bind to the GH1 domain of KLB: 22414 (also referred to as 414); 22401 (also referred to as 401); 22393 (also referred to as 393); 17067.

Antibodies that bind to the GH2 domain of KLB: 22532

Antibodies that bind to FGFR1c: ADI-19842 or 19842, ADI-19851 or 19851, ADI-19839 or 19839, and ADI-19863 or 19863.

When paired with a common light chain these antibodies are referred to with a P2 suffix (e.g., 22414P2, 22401P2, 22393P2, 17067P2, 22532P2, etc.).

The binding domains of the antibodies are depicted in FIG. 2.

Additional antibodies used in these studies include REGN4304, a bispecific anti-KLB, anti-FGFR1c antibody whose parental KLB binding arm is based on anti-GH

Additional constructs used in these studies include: REGN17067, a non-binding control antibody that binds to BetV1, a pollen antigen from Betula pendula, and REGN1438, which is 6His-FGF21.

### 7.1.2. Constant Domains

Antibody constructs comprising the constant domain and linker sequences as set out in Table 6 below were generated. The constructs are described in Table 7.

TABLE 6
Sequences of components of test and control constructs

| | | SEQ ID |
|---|---|---|
| Description | Amino Acid Sequence | NO. |
| hIgG1 (216-447; EU | EPKSCDKTHTCPPCPAPELLGGPSVFLFPPKPKDTLMISRTP | 45 |
| numbering) | EVTCVVVDVSHEDPEVKFNWYVDGVEVHNAKTKPREEQYNST | |
| hIgG1 hinge and Fc. | YRVVSVLTVLHQDWLNGKEYKCKVSNKALPAPIEKTISKAKG | |
| | QPREPQVYTLPPSRDELTKNQVSLTCLVKGFYPSDIAVEWES | |
| | NGQPENNYKTTPPVLDSDGSFFLYSKLTVDKSRWQQGNVFSC | |
| | SVMHEALHNHYTQKSLSLSPGK | |
| hIgG1 PVA: | EPKSCDKTHTCPPCPAPPVAGPSVFLFPPKPKDTLMISRTPE | 46 |
| Variant hIgG1 hinge | VTCVVVDVSHEDPEVKFNWYVDGVEVHNAKTKPREEQYNSTY | |
| and Fc, with IgG2- | RVVSVLTVLHQDWLNGKEYKCKVSNKALPAPIEKTISKAKGQ | |
| based hinge region and | PREPQVYTLPPSRDELTKNQVSLTCLVKGFYPSDIAVEWESN | |
| IgG1 CH2 and CH3 | GQPENNYKTTPPVLDSDGSFFLYSKLTVDKSRWQQGNVFSCS | |
| | VMHEALHNHYTQKSLSLSPGK | |
| hIgG1 PVA star: | EPKSCDKTHTCPPCPAPPVAGPSVFLFPPKPKDTLMISRTPE | 47 |
| Variant hIgG1 hinge | VTCVVVDVSHEDPEVKFNWYVDGVEVHNAKTKPREEQYNSTY | |
| and Fc, with IgG2- | RVVSVLTVLHQDWLNGKEYKCKVSNKALPAPIEKTISKAKGQ | |
| based hinge region and | PREPQVYTLPPSRDELTKNQVSLTCLVKGFYPSDIAVEWESN | |
| IgG1 CH2 and CH3 | GQPENNYKTTPPVLDSDGSFFLYSKLTVDKSRWQQGNVFSCS | |
| with star mutation | VMHEALHNRFTQKSLSLSPGK | |
| hIgG1s: | DKKVEPKSCDKTHTCPPCPAPPVAGPSVFLFPPKPKDTLMIS | 48 |
| Variant hIgG1 hinge | RTPEVTCVVVDVSQEDPEVQFNWYVDGVEVHNAKTKPREEQF | |
| and Fc, with IgG2- | NSTYRVVSVLTVLHQDWLNGKEYKCKVSNKGLPSSIEKTISK | |
| based hinge region and | AKGQPREPQVYTLPPSRDELTKNQVSLTCLVKGFYPSDIAVE | |
| IgG4CH2 and lgG1 | WESNGQPENNYKTTPPVLDSDGSFFLYSKLTVDKSRWQQGNV | |
| CH3 | FSCSVMHEALHNHYTQKSLSLSPGK | |
| hIgG1 N180G, also | EPKSCDKTHTCPPCPAPELLGGPSVFLFPPKPKDTLMISRTP | 49 |
| referred to as N297G: | EVTCVVVDVSHEDPEVKFNWYVDGVEVHNAKTKPREEQYGST | |
| Variant hIgG1 hinge | YRVVSVLTVLHQDWLNGKEYKCKVSNKALPAPIEKTISKAKG | |
| and Fc, with N180G | QPREPQVYTLPPSRDELTKNQVSLTCLVKGFYPSDIAVEWES | |
| mutation in depicted | NGQPENNYKTTPPVLDSDGSFFLYSKLTVDKSRWQQGNVFSC | |
| sequence(N297G by | SVMHEALHNHYTQKSLSLSPGK | |
| EU numbering) | | |
| hIgG2 (216-447; EU | ERKCCVECPPCPAPPVAGPSVFLFPPKPKDTLMISRTPEVTCVVVD | 50 |
| numbering) | VSHEDPEVQFNWYVDGVEVHNAKTKPREEQFNSTFRVVSVLTVVHQ | |
| hIgG2 hinge and Fc | DWLNGKEYKCKVSNKGLPAPIEKTISKTKGQPREPQVYTLPPSREE | |
| according to Uniprot | MTKNQVSLTCLVKGFYPSDISVEWESNGQPENNYKTTPPMLDSDGS | |
| P01859 | FFLYSKLTVDKSRWQQGNVFSCSVMHEALHNHYTQKSLSLSPGK | |
| hIgG2 (216-447; EU | ERKCCVECPPCPAPPVAGPSVFLFPPKPKDTLMISRTPEVTCVWD | 51 |
| numbering) | VSHEDPEVQFNWYVDGVEVHNAKTKPREEQFNSTFRWSVLTVVHQ | |
| Variant hIgG2 | DWLNGKEYKCKVSNKGLPAPIEKTISKTKGQPREPQVYTLPPSREE | |
| sequence with S to A | MTKNQVSLTCLVKGFYPSDIAVEWESNGQPENNYKTTPPMLDSDGS | |
| substitution at position | FFLYSKLTVDKSRWQQGNVFSCSVMHEALHNHYTQKSLSLSPGK | |
| 159 of depicted | | |
| sequence (DISVE to | | |
| DIAVE). Unless | | |
| indicated otherwise, the | | |
| variant hIgG2 sequence | | |
| was used in the | | |
| constructs exemplified | | |
| herein. | | |
| hIgG4 (216-447; EU | ESKYGPPCPSCPAPEFLGGPSVFLFPPKPKDTLMISRTPEVTCVVV | 52 |
| numbering): | DVSQEDPEVQFNWYVDGVEVHNAKTKPREEQFNSTYRVVSVLTVLH | |
| | QDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| hIgG4 S108P: | ESKYGPPCPPCPAPEFLGGPSVFLFPPKPKDTLMISRTPEVTCVVV | 53 |
| Variant hIgG4 hinge | DVSQEDPEVQFNWYVDGVEVHNAKTKPREEQFNSTYRVVSVLTVLH | |
| and Fc, with S108P | QDWLNGKEYKCKVSNKGLPSSIEKTISKAKGQPREPQVYTLPPSQE | |
| mutation (S228P by EU | EMTKNQVSLTCLVKGFYPSDIAVEWESNGQPENNYKTTPPVLDSDG | |
| numbering). | SFFLYSRLTVDKSRWQEGNVFSCSVMHEALHNHYTQKSLSLSLGK | |
| hIgG4s: | ESKYGPPCPPCPAPPVAGPSVFLFPPKPKDTLMISRTPEVTCVVVD | 54 |
| Variant hIgG4 hinge | VSQEDPEVQFNWYVDGVEVHNAKTKPREEQFNSTYRVVSVLTVLHQ | |
| and Fc, with IgG2- | DWLNGKEYKCKVSNKGLPSSIEKTISKAKGQPREPQVYTLPPSQEE | |
| based hinge region with | MTKNQVSLTCLVKGFYPSDIAVEWESNGQPENNYKTTPPVLDSDGS | |
| S108P mutation | FFLYSRLTVDKSRWQEGNVFSCSVMHEALHNHYTQKSLSLSLGK | |
| (S228P by EU | | |
| numbering), and IgG1 | | |
| CH2 and CH3 | | |
| G₄S: | GGGGS | 17 |
| 7-amino acid linker | GGGGSGG | 55 |
| 15-amino acid linker | 3x G₄S (i.e., GGGGSGGGGSGGGGS) | 56 |
| 30-amino acid linker | 6x G₄S (i.e., | 57 |
| | GGGGSGGGGSGGGGSGGGGSGGGGSGGGGS) | |
| IgG1 PVA_hinge-Fc | EPKSCDKTHTCPPCPAPPVAGPSVFLFPPKPKDTLMISRTPE | 58 |
| knob | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPSRDELTKNQVSLWCLVKGFYPSDIAVEWESN | |
| based hinge region and | GQPENNYKTTPPVLDSDGSFFLYSKLTVDKSRWQQGNVFSCS | |
| IgG1 CH2 and CH3 | VMHEALHNHYTQKSLSLSPGK | |
| with knob mutation | | |
| T366W | | |
| IgG1 PVA_hinge-Fc | EPKSCDKTHTCPPCPAPPVAGPSVFLFPPKPKDTLMISRTPE | 59 |
| knob_star | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPSRDELTKNQVSLWCLVKGFYPSDIAVEWESN | |
| based hinge region and | GQPENNYKTTPPVLDSDGSFFLYSKLTVDKSRWQQGNVFSCS | |
| IgG1 CH2 and CH3 | VMHEALHNRFTQKSLSLSPGK | |
| with knob mutation | | |
| T366W, and star | | |
| mutations H435R, | | |
| Y436F | | |
| IgG1 PVA_hinge-Fc | EPKSCDKTHTCPPCPAPPVAGPSVFLFPPKPKDTLMISRTPE | 60 |
| knob_Cys | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPCRDELTKNQVSLWCLVKGFYPSDIAVEWESN | |
| based hinge region and | GQPENNYKTTPPVLDSDGSFFLYSKLTVDKSRWQQGNVFSCS | |
| IgG1 CH2 and CH3 | VMHEALHNHYTQKSLSLSPGK | |
| with knob mutation | | |
| T366W, and Cys | | |
| mutation S354C | | |
| IgG1 PVA_hinge-Fc | EPKSCDKTHTCPPCPAPPVAGPSVFLFPPKPKDTLMISRTPE | 61 |
| knob_Cys_star | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPCRDELTKNQVSLWCLVKGFYPSDIAVEWESN | |
| based hinge region and | GQPENNYKTTPPVLDSDGSFFLYSKLTVDKSRWQQGNVFSCS | |
| IgG1 CH2 and CH3 | VMHEALHNRFTQKSLSLSPGK | |
| with knob mutation | | |
| T366W, Cys mutation | | |
| S354C mutation and | | |
| star mutations H435R, | | |
| Y436F | | |
| IgG1 PVA_hinge-Fc | EPKSCDKTHTCPPCPAPPVAGPSVFLFPPKPKDTLMISRTPE | 62 |
| Hole | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPSRDELTKNQVSLSCAVKGFYPSDIAVEWESN | |
| based hinge region and | GQPENNYKTTPPVLDSDGSFFLVSKLTVDKSRWQQGNVFSCS | |
| IgG1 CH2 and CH3 | VMHEALHNHYTQKSLSLSPGK | |
| with hole mutations | | |
| T366S, L368A, Y407V, | | |
| IgG1 PVA_hinge-Fc | EPKSCDKTHTCPPCPAPPVAGPSVFLFPPKPKDTLMISRTPE | 63 |
| Hole_star | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVYTLPPSRDELTKNQVSLSCAVKGFYPSDIAVEWESN | |
| based hinge region and | GQPENNYKTTPPVLDSDGSFFLVSKLTVDKSRWQQGNVFSCS | |
| IgG1 CH2 and CH3 | VMHEALHNRFTQKSLSLSPGK | |
| with hole mutations | | |
| T366S, L368A, Y407V, | | |
| and star mutations | | |
| H435R, Y436F | | |
| IgG1 PVA_hinge-Fc | EPKSCDKTHTCPPCPAPPVAGPSVFLFPPKPKDTLMISRTPE | 64 |
| Hole_Cys | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVCTLPPSRDELTKNQVSLSCAVKGFYPSDIAVEWESN | |
| based hinge region and | GQPENNYKTTPPVLDSDGSFFLVSKLTVDKSRWQQGNVFSCS | |
| IgG1 CH2 and CH3 | VMHEALHNHYTQKSLSLSPGK | |
| with hole mutations | | |
| T366S, L368A, Y407V, | | |
| and Cys mutation | | |
| S354C | | |
| IgG1 PVA_hinge-Fc | EPKSCDKTHTCPPCPAPPVAGPSVFLFPPKPKDTLMISRTPE | 65 |
| Hole_Cys_star | VTCVVVDVSHEDPEVKFNWYVDGVEVHNAKTKPREEQYNSTY | |
| Variant hIgG1 hinge | RVVSVLTVLHQDWLNGKEYKCKVSNKALPAPIEKTISKAKGQ | |
| and Fc, with IgG2- | PREPQVCTLPPSRDELTKNQVSLSCAVKGFYPSDIAVEWESN | |
| based hinge region and | GQPENNYKTTPPVLDSDGSFFLVSKLTVDKSRWQQGNVFSCS | |
| IgG1 CH2 and CH3 | VMHEALHNRFTQKSLSLSPGK | |
| with hole mutations | | |
| T366S, L368A, Y407V, | | |
| Cys mutation S354C | | |
| and star mutations | | |
| H435R, Y436F | | |

### 7.1.3. Description of Constructs

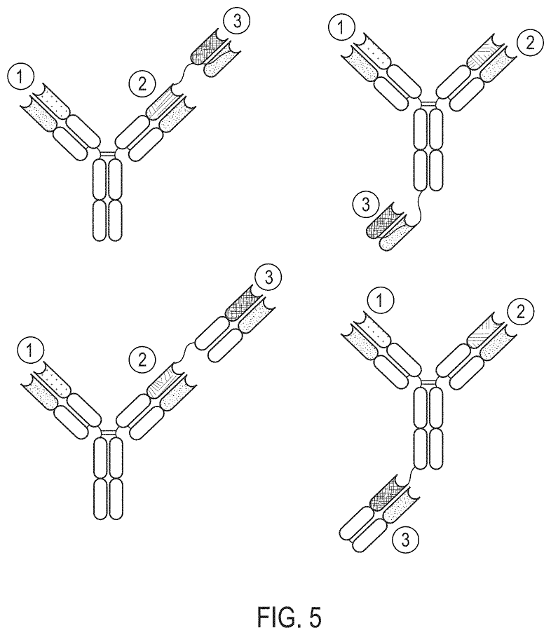

Test and control constructs included various bispecific and trispecific binding molecules, as set out below in Table 7, which provides descriptions of the various controls and test constructs utilized throughout the studies described herein. “ABS1 Target” in trispecific constructs refers to the target of the antigen binding module labeled “1” in the schematics of FIG. 5. “ABS2 Target” in trispecific constructs refers to the target of the antigen binding module labeled “2” in the schematics of FIG. 5. “ABS3 Target” in trispecific constructs refers to the target of the antigen binding module labeled “3” in the schematics of FIG. 5. The reference to “ABS3 linker length” refers to the length of the linker separating the antigen binding module labeled “3” in the schematics of FIG. 5 with the adjoining Fab or Fc domain, as applicable.

TABLE 7

| | | | | | | ABS3 | |
|---|---|---|---|---|---|---|---|
| | | | ABS1 | ABS2 | ABS3 | Linker | |
| Designation | Format | IgG isotype | Target | Target | Target | Length | Parental antibodies |
| REGN4355 | Std. 1 + 1 | IgG4 | GH1 | D3 | N/A | N/A | 22393P2 (GH1), ADI-19839 (D3) |
| | bispecific | | | | | | |
| REGN4366 | Std. 1 + 1 | IgG4 | GH1 | D3 | N/A | N/A | 22393P2 (GH1), ADI-19842 (D3) |
| | bispecific | | | | | | |
| REGN4370 | Std. 1 + 1 | IgG4 | GH1 | D3 | N/A | N/A | 22393P2 (GH1), ADI-19851 (D3) |
| | bispecific | | | | | | |
| REGN4376 | Std. 1 + 1 | IgG4 | GH1 | D3 | N/A | N/A | 22393P2 (GH1), ADI-19863 (D3) |
| | bispecific | | | | | | |
| REGN4304 | Std. 1 + 1 | IgG4s | GH2 | D2 | N/A | N/A | cKLB (GH2), cFGFR1c (D2) |
| | bispecific | | | | | | |
| F1K_scFv1 | 2 + 1 N-scFv | IgG1 K/H*, S-S | GH1 | D3 | GH1 | 15 | 22393P2 (GH1), ADI-19842 (D3), |
| | | | | | | | 22414P2 (GH1) |
| F1K_scFv2 | 2 + 1 N-scFv | IgG1 K/H*, S-S | GH1 | D3 | GH1 | 15 | 22393P2 (GH1), ADI-19842 (D3), |
| | | | | | | | 22401P2 (GH1) |
| F1K_scFv3 | 2 + 1 N-scFv | IgG1 K/H*, S-S | GH1 | D3 | GH2 | 15 | 22393P2 (GH1), ADI-19842 (D3), |
| | | | | | | | 22532P2 (GH2 |
| F1K_scFv4 | 2 + 1 N-scFv | IgG1 K/H*, S-S | GH1 | D3 | GH1 | 30 | 22393P2 (GH1), ADI-19842 (D3), |
| | | | | | | | 22414P2 (GH1) |
| F1K_scFv5 | 2 + 1 N-scFv | IgG1 K/H*, S-S | GH1 | D3 | GH1 | 30 | 22393P2 (GH1), ADI-19842 (D3), |
| | | | | | | | 22401P2 (GH1) |
| F1K_scFv6 | 2 + 1 N-scFv | IgG1 K/H*, S-S | GH1 | D3 | GH2 | 30 | 22393P2 (GH1), ADI-19842 (D3), |
| | | | | | | | 22532P2 (GH2) |
| F1K_scFv7 | 2 + 1 N-scFv | IgG1 K/H*, S-S | GH1 | D3 | GH1 | 45 | 22393P2 (GH1), ADI-19842 (D3), |
| | | | | | | | 22414P2 (GH1) |
| F1K_scFv8 | 2 + 1 N-scFv | IgG1 K/H*, S-S | GH1 | D3 | GH1 | 45 | 22393P2 (GH1), ADI-19842 (D3), |
| | | | | | | | 22401P2 (GH1) |
| F1K_scFv9 | 2 + 1 N-scFv | IgG1 K/H*, S-S | GH1 | D3 | GH2 | 45 | 22393P2 (GH1), ADI-19842 (D3), |
| | | | | | | | 22532P2 (GH2) |
| F1K_scFv10 | 2 + 1 C-scFv | IgG1 K/H*, S-S | GH1 | D3 | GH1 | 15 | 22393P2 (GH1), ADI-19842 (D3), |
| | | | | | | | 22414P2 (GH1) |
| F1K_scFv11 | 2 + 1 C-scFv | IgG1 K/H*, S-S | GH1 | D3 | GH1 | 15 | 22393P2 (GH1), ADI-19842 (D3), |
| | | | | | | | 22401P2 (GH1) |
| F1K_scFv12 | 2 + 1 C-scFv | IgG1 K/H*, S-S | GH1 | D3 | GH2 | 15 | 22393P2 (GH1), ADI-19842 (D3), |
| | | | | | | | 22532P2 (GH2) |
| F1K_scFv13 | 2 + 1 C-scFv | IgG1 K/H*, S-S | GH1 | D3 | GH1 | 30 | 22393P2 (GH1), ADI-19842 (D3), |
| | | | | | | | 22414P2 (GH1) |
| F1K_scFv14 | 2 + 1 C-scFv | IgG1 K/H*, S-S | GH1 | D3 | GH1 | 30 | 22393P2 (GH1), ADI-19842 (D3), |
| | | | | | | | 22401P2 (GH1) |
| F1K_scFv15 | 2 + 1 C-scFv | IgG1 K/H*, S-S | GH1 | D3 | GH2 | 30 | 22393P2 (GH1), ADI-19842 (D3), |
| | | | | | | | 22532P2 (GH2) |
| F1K_scFv16 | 2 + 1 C-scFv | IgG1 K/H*, S-S | GH1 | D3 | GH1 | 45 | 22393P2 (GH1), ADI-19842 (D3), |
| | | | | | | | 22414P2 (GH1) |
| F1K_scFv17 | 2 + 1 C-scFv | IgG1 K/H*, S-S | GH1 | D3 | GH1 | 45 | 22393P2 (GH1), ADI-19842 (D3), |
| | | | | | | | 22401P2 (GH1) |
| F1K_scFv18 | 2 + 1 C-scFv | IgG1 K/H*, S-S | GH1 | D3 | GH2 | 45 | 22393P2 (GH1), ADI-19842 (D3), |
| | | | | | | | 22532P2 (GH2) |
| F1k-scFv6-LK7 | 2 + 1 N-scFv | IgG1 K/H*, S-S | GH1 | D3 | GH2 | 7 | 22393P2 (GH1), ADI-19842 (D3), |
| | | | | | | | 22532P2 (GH2) |
| F1k-scFv6-LK15 | 2 + 1 N-scFv | IgG1 K/H*, S-S | GH1 | D3 | GH2 | 15 | 22393P2 (GH1), ADI-19842 (D3), |
| | | | | | | | 22532P2 (GH2) |
| F1k-scFv6-LK22 | 2 + 1 N-scFv | IgG1 K/H*, S-S | GH1 | D3 | GH2 | 22 | 22393P2 (GH1), ADI-19842 (D3), |
| | | | | | | | 22532P2 (GH2) |
| F1k-scFv6-LK30 | 2 + 1 N-scFv | IgG1 K/H*, S-S | GH1 | D3 | GH2 | 30 | 22393P2 (GH1), ADI-19842 (D3), |
| | | | | | | | 22532P2 (GH2) |
| F1k-scFv6-LK37 | 2 + 1 N-scFv | IgG1 K/H*, S-S | GH1 | D3 | GH2 | 37 | 22393P2 (GH1), ADI-19842 (D3), |
| | | | | | | | 22532P2 (GH2) |
| F1k-scFv6-LK45 | 2 + 1 N-scFv | IgG1 K/H*, S-S | GH1 | D3 | GH2 | 45 | 22393P2 (GH1), ADI-19842 (D3), |
| | | | | | | | 22532P2 (GH2) |
| F1k-scFv6-hG4 | 2 + 1 N-scFv | IgG4 K/H*, S-S | GH1 | D3 | GH2 | 30 | 22393P2 (GH1), ADI-19842 (D3), |
| | | | | | | | 22532P2 (GH2) |
| F1k-scFv6-HL | 2 + 1 N-scFv | IgG1 K/H*, S-S | GH1 | D3 | GH2 | 30 | 22393P2 (GH1), ADI-19842 (D3), |
| (heavy/light | | | | | | | 22532P2 (GH2) |
| chain swap) | | | | | | | |
| F1k-scFv19 | 2 + 1 N-scFv | IgG1 K/H*, S-S | GH1 | D3 | GH2 | 30 | 22477P2 (GH1), ADI-19842 (D3), |
| | | | | | | | 22532P2 (GH2) |
| F1k-scFv20 | 2 + 1 N-scFv | IgG1 K/H*, S-S | GH1 | D3 | GH2 | 30 | 22401P2 (GH1), ADI-19842 (D3), |
| | | | | | | | 22532P2 (GH2) |
| F1k-scFv6-RT1 | 2 + 1 N-scFv | IgG1 K/H*, N180G | GH1 | D3 | GH2 | 30 | 22393P2 (GH1), ADI-19842 (D3), |
| | | | | | | | 22532P2 (GH2) |
| F1k-scFv6-RT2 | 2 + 1 N-scFv | IgG1 K/H*, N180G | D3 | GH1 | GH2 | 30 | ADI-19842 (D3), 22393P2 (GH1), |
| | | | | | | | 22532P2 (GH2) |
| F1k-scFv6-RT3 | 2 + 1 N-scFv | IgG1 K/H*, N180G | GH1 | GH2 | D3 | 30 | 22393P2 (GH1), 22532P2 (GH2), |
| | | | | | | | ADI-19842 (D3) |
| F1k-scFv6-RT4 | 2 + 1 N-scFv | IgG1 K/H*, N180G | GH2 | GH1 | D3 | 30 | 22532P2 (GH2), 22393P2 (GH1), |
| | | | | | | | ADI-19842 (D3) |
| F1k-scFv6-RT5 | 2 + 1 N-scFv | IgG1 K/H*, N180G | D3 | GH2 | GH1 | 30 | ADI-19842 (D3), 22532P2 (GH2), |
| | | | | | | | 22393P2 (GH1) |
| F1k-scFv6-RT6 | 2 + 1 N-scFv | IgG1 K/H*, N180G | GH2 | D3 | GH1 | 30 | 22532P2 (GH2), ADI-19842 (D3), |
| | | | | | | | 22393P2 (GH1) |
| F1K_Fab1 | 2 + 1 N-Fab | IgG1 K/H*, S-S | GH1 | D3 | GH1 | 15 | 22393P2 (GH1), ADI-19842 (D3), |
| | | | | | | | 22414P2 (GH1) |
| F1K_Fab 2 | 2 + 1 N-Fab | IgG1 K/H*, S-S | GH1 | D3 | GH1 | 15 | 22393P2 (GH1), ADI-19842 (D3), |
| | | | | | | | 22401P2 (GH1) |
| F1K_Fab 3 | 2 + 1 N-Fab | IgG1 K/H*, S-S | GH1 | D3 | GH2 | 15 | 22393P2 (GH1), ADI-19842 (D3), |
| | | | | | | | 22532P2 (GH2) |
| F1K_Fab 4 | 2 + 1 N-Fab | IgG1 K/H*, S-S | GH1 | D3 | GH1 | 30 | 22393P2 (GH1), ADI-19842 (D3), |
| | | | | | | | 22414P2 (GH1) |
| F1K_Fab 5 | 2 + 1 N-Fab | IgG1 K/H*, S-S | GH1 | D3 | GH1 | 30 | 22393P2 (GH1), ADI-19842 (D3), |
| | | | | | | | 22401P2 (GH1) |
| F1K_Fab 6 | 2 + 1 N-Fab | IgG1 K/H*, S-S | GH1 | D3 | GH2 | 30 | 22393P2 (GH1), ADI-19842 (D3), |
| | | | | | | | 22532P2 (GH2) |
| F1K_Fab 7 | 2 + 1 N-Fab | IgG1 K/H*, S-S | GH1 | D3 | GH1 | 45 | 22393P2 (GH1), ADI-19842 (D3), |
| | | | | | | | 22414P2 (GH1) |
| F1K_Fab 8 | 2 + 1 N-Fab | IgG1 K/H*, S-S | GH1 | D3 | GH1 | 45 | 22393P2 (GH1), ADI-19842 (D3), |
| | | | | | | | 22401P2 (GH1) |
| F1K_Fab 9 | 2 + 1 N-Fab | IgG1 K/H*, S-S | GH1 | D3 | GH2 | 45 | 22393P2 (GH1), ADI-19842 (D3), |
| | | | | | | | 22532P2 (GH2) |
| F1K_Fab 10 | 2 + 1 N-Fab | IgG1 K/H*, S-S | GH1 | D3 | GH1 | 15 | 22393P2 (GH1), ADI-19842 (D3), |
| | | | | | | | 22414P2 (GH1) |
| F1K_Fab 11 | 2 + 1 N-Fab | IgG1 K/H*, S-S | GH1 | D3 | GH1 | 15 | 22393P2 (GH1), ADI-19842 (D3), |
| | | | | | | | 22401P2 (GH1) |
| F1K_Fab12 | 2 + 1 N-Fab | IgG1 K/H*, S-S | GH1 | D3 | GH2 | 15 | 22393P2 (GH1), ADI-19842 (D3), |
| | | | | | | | 22532P2 (GH2) |
| F1K_Fab13 | 2 + 1 C-Fab | IgG1 K/H*, S-S | GH1 | D3 | GH1 | 30 | 22393P2 (GH1), ADI-19842 (D3), |
| | | | | | | | 22414P2 (GH1) |
| F1K_Fab14 | 2 + 1 C-Fab | IgG1 K/H*, S-S | GH1 | D3 | GH1 | 30 | 22393P2 (GH1), ADI-19842 (D3), |
| | | | | | | | 22401P2 (GH1) |
| F1K_Fab15 | 2 + 1 C-Fab | IgG1 K/H*, S-S | GH1 | D3 | GH2 | 30 | 22393P2 (GH1), ADI-19842 (D3), |
| | | | | | | | 22532P2 (GH2) |
| F1K_Fab16 | 2 + 1 C-Fab | IgG1 K/H*, S-S | GH1 | D3 | GH1 | 45 | 22393P2 (GH1), ADI-19842 (D3), |
| | | | | | | | 22414P2 (GH1) |
| F1K_Fab17 | 2 + 1 C-Fab | IgG1 K/H*, S-S | GH1 | D3 | GH1 | 45 | 22393P2 (GH1), ADI-19842 (D3), |
| | | | | | | | 22401P2 (GH1) |
| F1K_Fab18 | 2 + 1 C-Fab | IgG1 K/H*, S-S | GH1 | D3 | GH2 | 45 | 22393P2 (GH1), ADI-19842 (D3), |
| | | | | | | | 22532P2 (GH2) |
| KFB-FabN | 2 + 1 N-Fab | IgG1 K/H*, S-S | BetV1 | D3 | GH1 | 30 | 17067P2 (BetV1), ADI-19842 (D3), |
| | | | | | | | 22414P2 (GH1) |
| KBK-scFvN | 2 + 1 N-scFv | IgG1 K/H*, S-S | GH1 | BetV1 | GH1 | 30 | 22393P2 (GH1), 17067P2 (BetV1), |
| | | | | | | | 22414P2 (GH1) |
| F1k-scFv6LK7 | 2 + 1 N-scFv | IgG1 PVA | GH1 | D3 | GH2 | 7 | 22393P2 (GH1), ADI-19842 (D3), |
| IgG1 PVA | | | | | | | 22532P2 (GH2) |
| F1k-scFv6LK7 | 2 + 1 N-scFv | IgG4 S108P | GH1 | D3 | GH2 | 7 | 22393P2 (GH1), ADI-19842 (D3), |
| IgG4 S108P | | | | | | | 22532P2 (GH2) |
| F1k-scFv6LK15 | 2 + 1 N-scFv | IgG1 PVA | GH1 | D3 | GH2 | 15 | 22393P2 (GH1), ADI-19842 (D3), |
| IgG1 PVA | | | | | | | 22532P2 (GH2) |
| F1k-scFv6LK15 | 2 + 1 N-scFv | IgG4 S108P | GH1 | D3 | GH2 | 15 | 22393P2 (GH1), ADI-19842 (D3), |
| IgG4 S108P | | | | | | | 22532P2 (GH2) |
| F1k-scFv6 IgG1 | 2 + 1 N-scFv | IgG1 PVA | GH1 | D3 | GH2 | 30 | 22393P2 (GH1), ADI-19842 (D3), |
| PVA | | | | | | | 22532P2 (GH2) |
| F1k-scFv6, IgG4s | 2 + 1 N-scFv | IgG4 S108P | GH1 | D3 | GH2 | 30 | 22393P2 (GH1), ADI-19842 (D3), |
| S108P | | | | | | | 22532P2 (GH2) |
| F1k-scFv6, IgG1 | 2 + 1 N-scFv | IgG1 N180G | GH1 | D3 | GH2 | 15 | 22393P2 (GH1), ADI-19842 (D3), |
| N180G | | | | | | | 22532P2 (GH2) |
| F1k-scFv6 IgG1 | 2 + 1 N-scFv | IgG1 | GH1 | D3 | GH2 | 30 | 22393P2 (GH1), ADI-19842 (D3), |
| | | | | | | | 22532P2 (GH2) |
| F1k-Fab6, IgG1 | 2 + 1 N-Fab | IgG1 PVA | GH1 | D3 | GH2 | 30 | 22393P2 (GH1), ADI-19842 (D3), |
| PVA | | | | | | | 22532P2 (GH2) |
| F1k-Fab6, IgG4s | 2 + 1 N-Fab | IgG4s S108P | GH1 | D3 | GH2 | 30 | 22393P2 (GH1), ADI-19842 (D3), |
| S108P | | | | | | | 22532P2 (GH2) |
| F1K-Fab6 IgG1 | 2 + 1 N-Fab | IgG1 N180G | GH1 | D3 | GH2 | 30 | 22393P2 (GH1), ADI-19842 (D3), |
| N180G | | | | | | | 22532P2 (GH2) |
| F1K-Fab6 IgG1 | 2 + 1 N-Fab | IgG1 | GH1 | D3 | GH2 | 30 | 22393P2 (GH1), ADI-19842 (D3), |
| | | | | | | | 22532P2 (GH2) |
| IgG1 PVA | 2 + 1 N-Fab | IgG1 PVA | GH1 | D3 | GH2 | 15 | 22393P2 (GH1), ADI-19842 (D3), |
| | | | | | | | 22532P2 (GH2) |
| IgG4s S108P | 2 + 1 N-Fab | IgG4s S108P | GH1 | D3 | GH2 | 15 | 22393P2 (GH1), ADI-19842 (D3), |
| | | | | | | | 22532P2 (GH2) |

| | | | |
|---|---|---|---|
| IgG4s | Trispecific | IgG4s | Irrelevant targets for IgG4s controls |

### 7.2. Example 2: Evaluating KLB and FGFR1c Antibodies in Bispecific Formats

### 7.2.1. Cloning and Expression of Bispecific Binding Molecules and Control Binders

Bispecific binding molecules containing the binding domains of antibodies identified in Example 1 were generated using an IgG4 Fc and star mutations to select for the correctly paired heterodimers as shown in Table 8 below:

TABLE 8

| | Anti-FCFR1c(IgG4*) | Anti-KLB (hIgG4) |
|---|---|---|
| REGN4366 | ADI-19842 | 22393 (22393 heavy chain/ |
| | | P2 universal light chain) |
| REGN4370 | ADI-19851 | 22393 (22393 heavy chain/ |
| | | P2 universal light chain) |
| REGN4355 | ADI-19839 | 22393 (22393 heavy chain/ |
| | | P2 universal light chain) |
| REGN4376 | ADI-19863 | 22393 (22393 heavy chain/ |
| | | P2 universal light chain) |

DNA fragments encoding KLB or FGFR1c VH and VL domains via direct DNA synthesis or subcloning were inserted into mammalian expression vectors containing human IgG4 or human IgG4 backbone with star mutations (H435R, Y436F, EU numbering), via either NEBuilder HiFi DNA Assembly Kit (New England BioLabs Inc.) or restriction digest and ligation following standard molecular cloning protocols provided by New England BioLabs Inc. CHO stable expression cell lines were generated. The mammalian expression and purification using protein A affinity, anti star affinity and size-exclusion chromatography were used to produce and purify the bispecifics for analysis.

The cloning, expression and purification for REGN4304 are similar to the generation of bispecifics as described except the following differences: 1. the VH domain for each KLB and FGFR1c half antibodies was inserted into human IgG4 Fc with knob mutations (S354C, T366W, EU numbering), and human IgG4 Fc with both hole (Y349C, T366S, L368A, Y407V) and star (H435R, Y436F) mutations. 2. Half antibody targeting FGFR1c or KLB was expressed separately and assembled via redox annealing as described (Williams et al., 2015, Biocatalysts and Bioreactor Design (31)-5)

The cloning, expression and purification for REGN1438 are similar to the generation of bispecifics as described except the following differences: 1. Human FGF21 (H29-S209, L174P) with an N-terminal hexa His tag (SEQ ID NO: 42) was inserted into an expression vector; 2. HisTrap affinity chromatography and size exclusion chromatography were used for purification.

### 7.2.2. Reporter Assay for FGFR1c/KLB Activation

Antibodies were tested for their agonist activities using HEK293.SREluc.hFGFR1c/hKLB cells that stably expressed human FGFR1c and KLB as well as a luciferase reporter gene under the control of a promoter containing serum responsive elements (SRE). Recombinant human FGF21 with 6×His tag (SEQ ID NO: 42) was used as a positive control, with the maximum reporter activity obtained from FGF21 defined as 100% activity. Cells were treated with each antibody or 6×His-FGF21 for 6 hours, and then subjected to luciferase assays. The percent activity induced by individual antibodies was normalized against the maximum activity by FGF21. Dose-response assays were performed to determine EC50. The anti FelD1 isotype control antibody, REGN1945, was used as a negative control.

### 7.2.3. Results

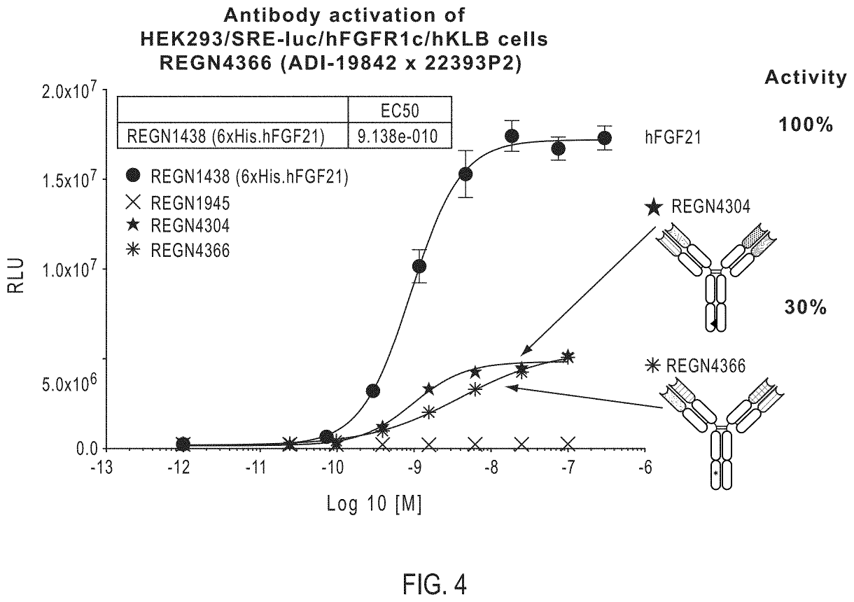

The results of the dose response assays are shown in FIG. 4. As shown in the figure, the activity of the bispecific binding molecules was almost an order of magnitude lower than FGF21 in activating KLB-FGFR1c.

### 7.3. Example 3: Evaluation of FGFR1c/KLB Activation by Trispecific Binding Molecules

### 7.3.1. Background

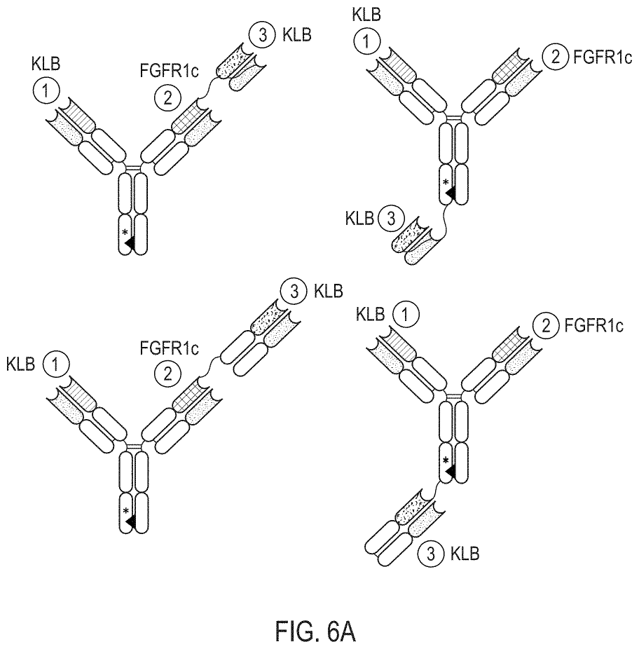

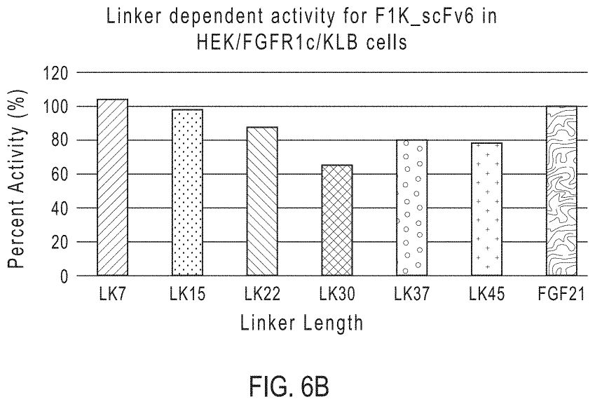

Trispecific binding molecules that bound to both the GH1 and GH2 domains of KLB in addition to were generated by the addition of an additional binding domain to REGN4366 in an attempt to increase its agonism of the FGFR1c/KLB co-receptor complex. REGN4366 is a bispecific binding molecule targeting the GH1 domain of KLB and the D3 domain of FGFR1c. A GH2 binding arm in the form of a Fab or scFv was added at different locations in the molecule as shown in FIG. 6A and the linker length between the REGN4366 portion of the molecule and the GH2 binding arm varied from 3 to 6 to 9 repeats of the G4S linker (SEQ ID NO: 43) (i.e., ranging from 15 to 45 amino acids).

### 7.3.2. Cloning and Expression of Trispecific Binding Molecules

DNA fragments encoding (i) KLB or FGFR1c or BetV1 scFvs in the orientation of VL (with a 100C mutation, Kabat numbering), linker (4×G₄S (SEQ ID NO: 44)), and VH (with a 44C mutation, Kabat numbering), followed by linkers of varied lengths for connecting the scFvs to a FGFR1c binding Fab, (ii) a KLB or FGFR1c or BetV1 binding Fab, and (iii) IgG1 Fc domains with knob-forming mutations (S354C, T366W, EU numbering), hole-forming mutations (Y349C, T366S, L368A, Y407V, EU numbering), a glycosylation mutation (N297G, EU numbering) and Star mutations (H435R, Y436F, EU numbering) were synthesized by Integrated DNA Technologies, Inc. (San Diego, Calif.), GenScript (Piscataway, N.J.) or Life Technologies (Carlsbad, Calif.).

Mammalian expression vectors for individual heavy chains were created by either NEBuilder HiFi DNA Assembly Kit (New England BioLabs Inc.) or restriction digest followed by ligation following standard molecular cloning protocols provided by New England BioLabs Inc. Some DNA fragments were made as ready to use constructs in pcDNA3.4 Topo expression system from Life Technologies (Carlsbad, Calif.). For expressing molecules depicted in FIG. 6A and listed in Table 9A, DNAs of heavy chains (“Hc1-Knob” and “Hc2-Hole*”) and universal light chain were co-transfected into Expi293 cells (ThermoFisher Scientific) following the manufacturer's protocol. 50 ml of cell culture medium was harvested and processed for purification via a HiTrap Protein A FF column (GE Healthcare). For functional confirmation, selected MBMs were scaled up to 200 ml and subject to a series of purification procedures including size exclusion chromatography as the final step.

### 7.3.3. Evaluation of the Activity and Assembly of the Trispecific Binding Molecules

The activity of the trispecific binding molecules was evaluated in a reporter assay as described in Section 7.2.2.

The assembly of the trispecific binding molecules was assayed by high-throughput analysis on a Cliper LabChip GX as per the manufacturer's protocol (Perkin Elmer, Waltham, Mass.). Briefly, the sample buffer was prepared by mixing 7 ml of HT protein express sample buffer with either 240 μl BME (reducing) or 25 mM iodoacetamide (IAM, for non-reducing assay). Samples were normalized to 0.5 mg/ml with sample buffer and then heated at 70° C. for 10 minutes. 70 μl of water was added to each sample before loading onto the instrument. The chip was prepared according to the manufacturer's instruction. Electropherogram of the samples were analyzed using the LabChip GX software. Peaks from non-reduced electropherogram indicate the % intact antibody.

### 7.4. Results

Table 9A below shows the percentage of assembly vs. percent activity for the various trispecific molecules and Table 9B below shows the activity of trispecific 2+1 N-scFv molecules with different linker lengths. FIG. 6B is a bar chart the data in Table 9B.

TABLE 9A

| Format | Molecule | Percent Assembly % | Percent activity % |
|---|---|---|---|
| N-scFv | F1K_scFv1 | 98.4 | 5 |
| N-scFv | F1K_scFv2 | 94.3 | 6.2 |
| N-scFv | F1K_scFv3 | 100 | 28.9 |
| N-scFv | F1K_scFv4 | 78.3 | 4.7 |
| N-scFv | F1K_scFv5 | 83.3 | 3 |
| N-scFv | F1K_scFv6 | 100 | 54.1 |
| N-scFv | F1K_scFv7 | 94.3 | 5.5 |
| N-scFv | F1K_scFv8 | 77.2 | 4.2 |
| N-scFv | F1K_scFv9 | 100 | 20.7 |
| N-scFv | F1K_scFv10 | 100 | 34.6 |
| N-scFv | F1K_scFv11 | 60.4 | 31.5 |
| N-scFv | F1K_scFv12 | 73.6 | 60.8 |
| C-scFv | F1K_scFv13 | 100 | 28.8 |
| C-scFv | F1K_scFv14 | 25 | 37.2 |
| C-scFv | F1K_scFv15 | 32.6 | 59.1 |
| C-scFv | F1K_scFv16 | 29.7 | 14.7 |
| C-scFv | F1K_scFv17 | 10.7 | 35.9 |
| C-scFv | F1K_scFv18 | 43.6 | 47.8 |
| N-Fab | F1K_Fab1 | 100 | 2.2 |
| N-Fab | F1K_Fab2 | 79.2 | 3.5 |
| N-Fab | F1K_Fab3 | 100 | 31.5 |
| N-Fab | F1K_Fab4 | 100 | 2.7 |
| N-Fab | F1K_Fab5 | 97.3 | 5.5 |
| N-Fab | F1K_Fab6 | 93.6 | 38.2 |
| N-Fab | F1K_Fab7 | 19.2 | 8.2 |
| N-Fab | F1K_Fab8 | 81 | 8 |
| N-Fab | F1K_Fab9 | 93.9 | 39.3 |
| C-Fab | F1K_Fab10 | 93.5 | 11.5 |
| C-Fab | F1K_Fab11 | 100 | 18.4 |
| C-Fab | F1K_Fab12 | 100 | 25.5 |
| C-Fab | F1K_Fab13 | 51.3 | 15.9 |
| C-Fab | F1K_Fab14 | 39.8 | 23.6 |
| C-Fab | F1K_Fab15 | 88.2 | 29.9 |
| C-Fab | F1K_Fab16 | 100 | 13.7 |
| C-Fab | F1K_Fab17 | 43.8 | 23.5 |
| C-Fab | F1K_Fab18 | 27.2 | 37.8 |
| REGN1945 | REGN1945 | 100 | 0 |

TABLE 9B

| Linker length | Percent Activity % |
|---|---|
| LK7 | 104.5 |
| LK15 | 97.5 |
| LK22 | 87.9 |
| LK30 | 66.4 |
| LK37 | 80.4 |
| LK45 | 78.7 |
| FGF21 | 100 |

The results indicate that the incorporation of the additional domain at the N-terminus (in the 2+1 N-scFv or 2+1 N-Fab trispecific binding molecule (TBM) formats) offers better assembly than incorporation of the additional binding domains at the C-terminus (in the 2+1 C-scFv or 2+1 C-Fab trispecific binding molecule (TBM) formats), whereas incorporation of an additional binding domain at the C-terminus in the 2+1 C-scFv resulted in greater activity.

### 7.5. Example 4: Comparison of the Agonistic Activity of Trispecific vs. Bispecific Binding Molecules

### 7.5.1. Materials & Methods

The agonistic activity of the 2+1 N-scFv (F1K_scFv6) and 2+1 N-Fab (F1K_Fab6) trispecific molecules described in Example 3 containing a was compared to the agonistic activity of the bispecific molecules described in Example 2 (REGN4304 and REGN4366) using the reporter assay described in Section 7.2.2.

HEK293.FGFR1c knock out cells were stably over-expressed with FGFR1c or KLB or FGFR1c+KLB. Cells were cultured in DMEM (Gibco, USA) supplemented with 10% of FBS (Gibco, USA) under standard conditions (37° C. in humidified atmosphere containing 5% CO2). For the Flow binding assay, 1×10⁵ cells/100 μL/well were seeded in a 96 well plate. Ca/Mg free PBS, supplemented with 1% FBS was used as staining buffer for antibody dilutions and subsequent washes. Cells were incubated with specified amount of primary antibody for 30 min at 4° C. After two washes, secondary antibody (F(ab′)₂ Fcγ fragment specific, Jackson immune research, 109-136-098) staining was performed for 30 min at 4° C. After subsequent washes cells were fixed in 2% paraformaldehyde for 30 min at room temperature. Fixed cells were washed and resuspended in 200 μL of staining buffer for flow cytometric analysis. A minimum of 10,000 single cells per sample were acquired on flow cytometer (Fortessa) and data were analyzed & Max MFI was calculated with FlowJo program. Graphs were created using Graphpad Prism software.

### 7.5.2. Results

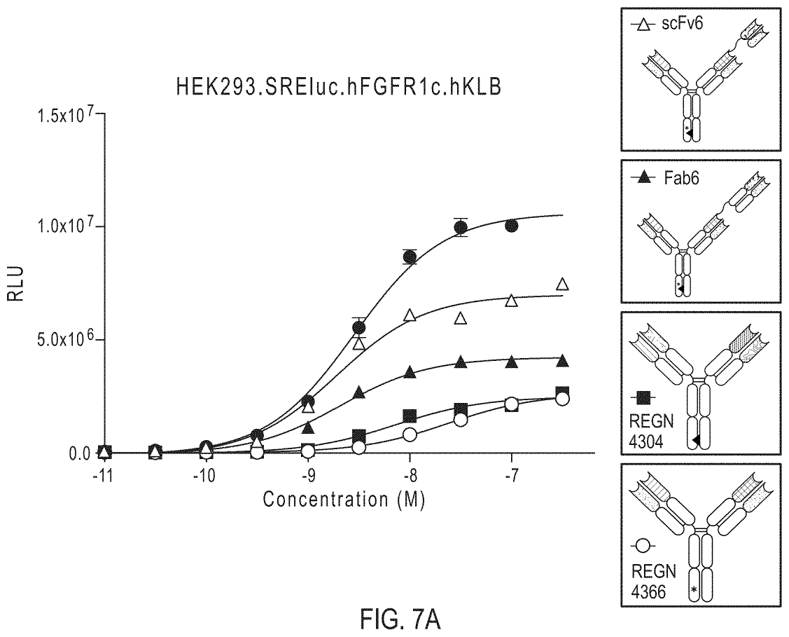

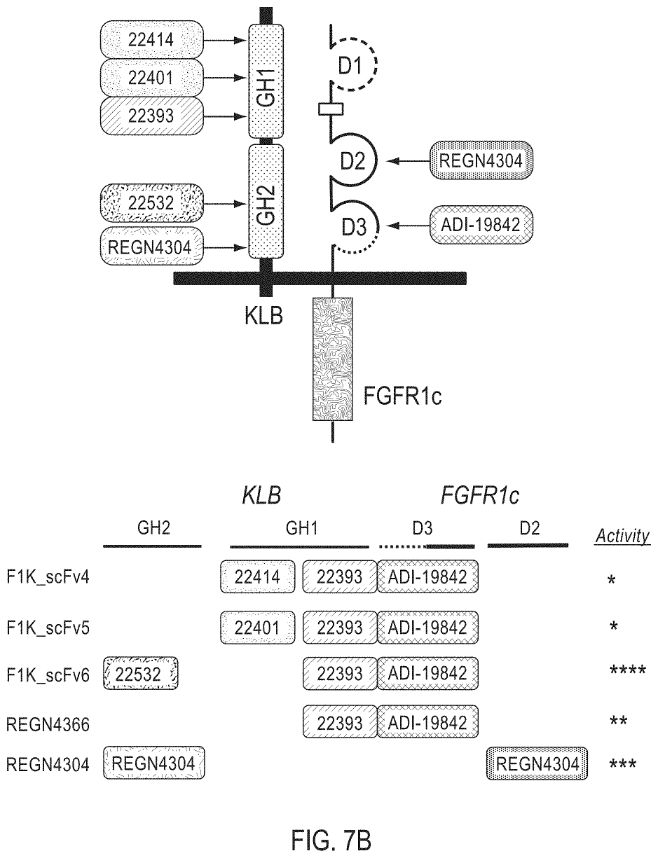

The results are shown in FIG. 7A, Table 10 (% activity in reporter assay) and Table 11 (binding affinity to KLB and FGFR1c) below.

TABLE 10

| Molecule | Description | % Act | EC50(M) |
|---|---|---|---|
| REGN1438 | 6His-FGF21 | 100 | 2.9E−09 |
| REGN4366 | Bispecific Binding Molecule | 24.8 | 2.4E−08 |
| F1K_scFv6 | 2 + 1 N-scFv | 65.9 | 1.9E−09 |
| F1K_Fab6 | 2 + 1 N-Fab | 39.9 | 2.1E−09 |
| REGN4304 | Bispecific Binding Molecule | 23.5 | 7.2E−09 |

TABLE 11

| | hKLB binding | hFGFR1c binding |
|---|---|---|
| Molecule | KD (M) | KD (M) |
| F1K_scFv6 | 1.47E−11 | 2.11E−06 |
| F1K_Fab6 | 1.18E−11 | 2.29E−06 |
| REGN4304 | 4.40E−11 | 1.09E−07 |
| REGN4366 | 2.55E−08 | 5.39E−07 |

FIG. 7B depicts the binding of the bi- and trispecific binding molecules to FGFR1c and KLB. Without being bound by theory, it is believed that the data of this Example shows that bi-epitopic engagement of hKLB improves antibody-mediated KLB/FGFR1c receptor complex interaction and potential cell surface clustering.

### 7.6. Example 5: Optimization of Trispecific Format

Three rounds of screening were conducted to optimize for the activity of the trispecific binding molecules.

In the first round of screening, the GH2 binding portion was substituted and the linker length separated the GH2-binding portion and the remainder of the binding molecule was varied from 15 to 45 amino acids. The molecules were constructed and expressed as described in Section 7.3.2 and the resulting molecules evaluated in the reporter assay described in Section 7.2.2. The results are shown in Table 12 below:

TABLE 12

| | 1 | 2 | 3 | Linker | % Act | EC50 |
|---|---|---|---|---|---|---|
| REGN1945 | IgG4 Isotype Control | | | | | |
| REGN1438 | 6His-FGF21 | | | | 100 | 3.4E−09 |
| F1K_Fab1 | 22393 | ADI-19842 | 22414 | 15 | 2.2 | 5.0E−10 |
| F1K_Fab2 | 22393 | ADI-19842 | 22401 | 15 | 3.5 | 1.9E−09 |
| F1K_Fab3 | 22393 | ADI-19842 | 22532 | 15 | 31.5 | 6.5E−10 |
| F1K_Fab4 | 22393 | ADI-19842 | 22414 | 30 | 2.7 | 8.5E−10 |
| F1K_Fab5 | 22393 | ADI-19842 | 22401 | 30 | 5.5 | 1.3E−09 |
| F1K_Fab6 | 22393 | ADI-19842 | 22532 | 30 | 38.2 | 6.2E−10 |
| F1K_Fab7 | 22393 | ADI-19842 | 22414 | 45 | 8.2 | 3.7E−08 |
| F1K_Fab8 | 22393 | ADI-19842 | 22401 | 45 | 8.0 | 3.6E−09 |
| F1K_Fab9 | 22393 | ADI-19842 | 22532 | 45 | 39.3 | 1.8E−09 |
| KFB_FabN | 17067 | ADI-19842 | 22414 | 30 | 1.8 | 8.3E−10 |
| F1K_scFv1 | 22393 | ADI-19842 | 22414 | L20H15 | 5.0 | 3.9E−09 |
| F1K_scFv2 | 22393 | ADI-19842 | 22401 | L20H15 | 6.2 | 3.7E−08 |
| F1K_scFv3 | 22393 | ADI-19842 | 22532 | L20H15 | 28.9 | 7.9E−09 |
| F1K_scFv4 | 22393 | ADI-19842 | 22414 | L20H30 | 4.7 | 3.5E−09 |
| F1K_scFv5 | 22393 | ADI-19842 | 22401 | L20H30 | 3.0 | 4.7E−08 |
| F1K_scFv6 | 22393 | ADI-19842 | 22532 | L20H30 | 54.1 | 9.8E−10 |
| F1K_scFv7 | 22393 | ADI-19842 | 22414 | L20H45 | 5.5 | 3.6E−09 |
| F1K_scFv8 | 22393 | ADI-19842 | 22401 | L20H45 | 4.2 | 4.2E−08 |
| F1K_scFv9 | 22393 | ADI-19842 | 22532 | L20H45 | 20.7 | 2.4E−09 |
| KBK_scFvN | 22393 | 17067 | 22414 | L20H30 | 2.1 | 1.2E−09 |

### 7.7. Example 6: Further Optimization of Trispecific Format

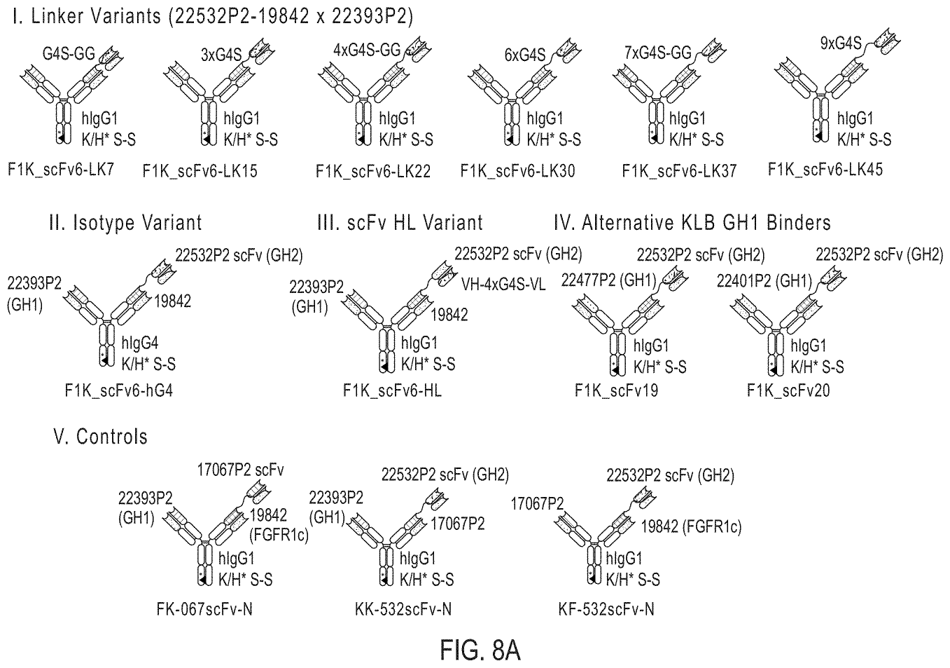

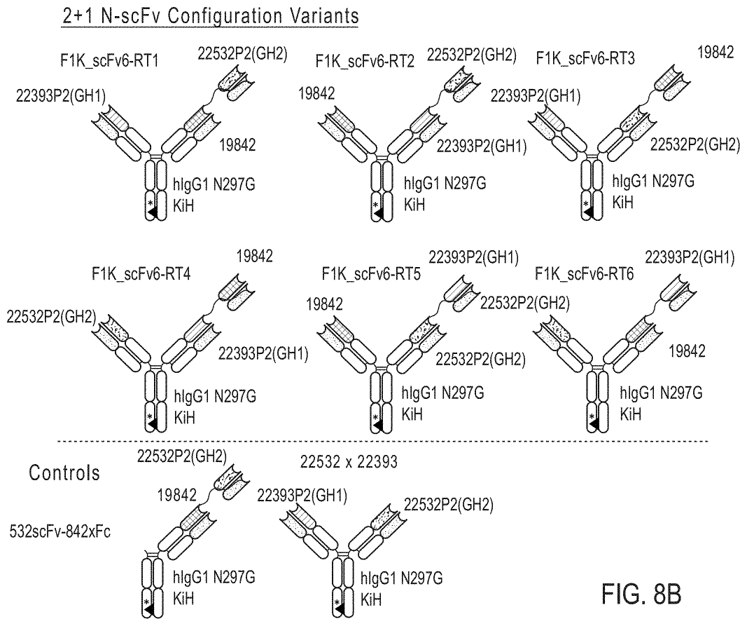

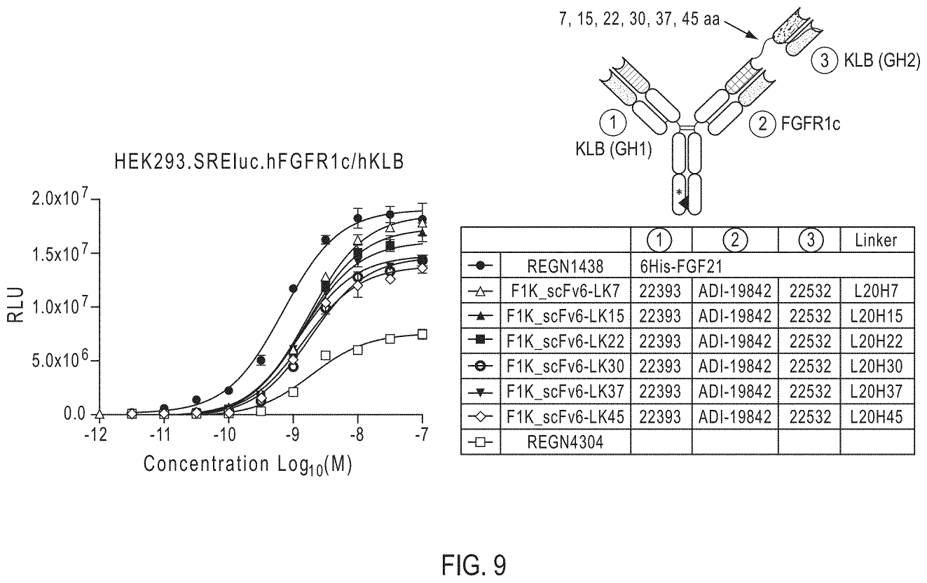

In further rounds of screening, the variants shown in FIG. 8A (with, inter alia, linker length variants) and FIG. 8B (with the GH1, GH2 and FGFR1c domains reconfigured) were evaluated in the reporter assay described in Section 7.2.2. The results for the variants in linker length between the domains designated 2 and 3 are shown in FIG. 9 and Table 13 below:

TABLE 13

| | 1 | 2 | 3 | Linker | EC50 | % Act |
|---|---|---|---|---|---|---|
| REGN1438 | 6His-FGF21 | | | | 5.7E−10 | 100.0 |
| F1K_scFv6-LK7 | 22393 | ADI-19842 | 22532 | L20H7 | 1.7E−09 | 104.5 |
| F1K_scFv6-LK15 | 22393 | ADI-19842 | 22532 | L20H15 | 1.6E−09 | 97.5 |
| F1K_scFv6-LK22 | 22393 | ADI-19842 | 22532 | L20H22 | 1.3E−09 | 87.9 |
| F1K_scFv6-LK30 | 22393 | ADI-19842 | 22532 | L20H30 | 2.0E−09 | 84.2 |
| F1K_scFv6-LK37 | 22393 | ADI-19842 | 22532 | L20H37 | 1.1E−09 | 80.4 |
| F1K_scFv6-LK45 | 22393 | ADI-19842 | 22532 | L20H45 | 1.5E−09 | 78.7 |
| REGN4304 | | | | | 1.8E−09 | 42.0 |

### 7.8. Example 7: Activation of FGFR1c Signaling in HEK293 Cells

### 7.8.1. Materials & Methods

HEK293.SREluc.hFGFR1c.hKLB stable cell line was generated by sequentially transfecting HEK293 cells with SRE-luciferase reporter, full length human FGFR1c, and full length human KLB plasmids. For western blot analysis, HEK293.SREluc.hFGFR1c.hKLB cells were plated in a 6-well plate, and cultured overnight in complete media containing 10% fetal bovine serum (FBS). The culture media was changed to Opti-MEM reduced serum medium (ThermoFisher, USA) supplemented with 0.1% FBS. Approximately 24 hr later, diluted ligands were added to the cells to final 1 nM or 10 nM concentrations. After a 15 minute treatment, cells were washed with cold PBS, and then lysed in RIPA lysis buffer (150 mMTris/HCl, pH 7.4, 50 mM NaCl, 1% NP-40 and 0.1% Tween 20). Total cell lysates were resolved by SDS-PAGE, and transferred onto PVDF membranes. For western blot analysis, the following primary antibodies were used: total ERK (Cell Signaling, 9102), phospho-ERK (Cell Signaling, 9101), PLC-gamma (Cell Signaling, 5690), phosphor-PLCgamma (Cell Signaling, 2821). For luciferase assays, HEK293.SREluc.hFGFR1c.hKLB cells were plated in a 384-well plate, and cultured overnight in complete media containing 10% fetal bovine serum (FBS). The culture media was changed to Opti-MEM reduced serum medium (ThermoFisher, USA) supplemented with 0.1% FBS. Approximately 24 hr later, cells were treated with serially diluted ligands for 6 hr, and then subjected to luciferase assay using ONE-Glo™ Luciferase Assay System (Promega, USA), according to the manufacturer's instructions.

### 7.8.2. Results

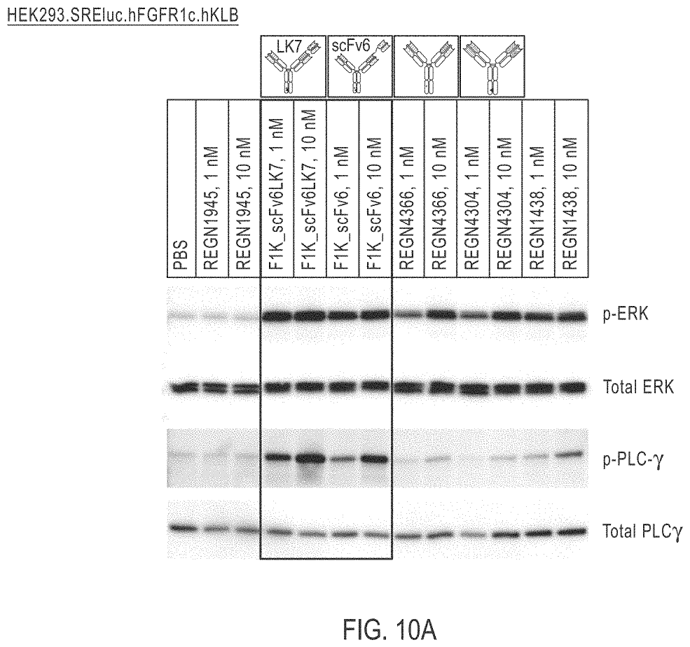

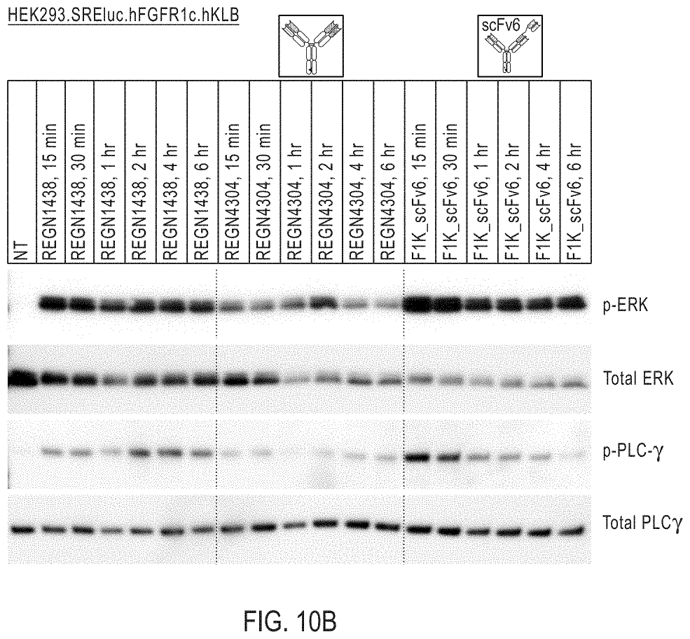

To determine the agonist activity of F1K_scFv6 and F1K_scFv6LK7, we treated HEK293.SREluc.hFGFR1c.hKLB cells that stably expressing human FGFR1c and human KLB, and measured ERK and PLC-gamma phosphorylation, which are induced by the activated FGFR1c (FIG. 10A). Both F1K_scFv6LK7 and F1K_scFv6LK7 strongly induced ERK and PLC-gamma phosphorylation at both 1 nM and 10 nM concentrations. Notably, phospho-ERK and phospho-PLC-gamma levels in F1K_scFv6 or F1K_scFv6LK7 treated cells were markedly higher than those in cells treated with corresponding concentrations of the parental bispecific antibody (REGN4366), an FGFR1/KLB agonist bispecific antibody (REGN4304), or recombinant human FGF21 (REGN1438).

To assess the time course of FGFR1c activation by F1K_scFv6 treatment, HEK293.SREluc.hFGFR1c.hKLB cells were treated with ligands for varying times, and harvested for western blot analysis (FIG. 10B). ERK activation measured by phospho-ERK levels was observed as early as 15 min following treatments with REGN1438, REGN4304, or F1K_scFv6, which persisted for up to 6 hours. F1K_scFv6 showed higher phospho-ERK levels compared with REGN1438 or REGN4304 throughout the time course of the treatment. F1K_scFv6 strongly induced phospho-PLCgamma at 15 min time point, which was then gradually decreased over time.

### 7.9. Example 8: Activation of ERK in Adipocytes

### 7.9.1. Materials & Methods

Subcutaneous human preadipocytes were obtained from Zen-Bio, Inc., and maintained in a 6-well plate in a Preadipocyte Medium provided by Zen-Bio. Preadipocytes were differentiated into adipocytes by culturing confluent preadipocytes in Adipocyte Differentiation Medium for 14 days. For western blot analysis, differentiated adipocytes were pretreated with Opti-MEM reduced serum medium (ThermoFisher, USA) supplemented with 0.1% FBS for 4 hours, and then treated with drugs for 15 minutes. Cells were washed with cold PBS, and then lysed in RIPA buffer for western blotting analysis.

Differentiated human subcutaneous adipocytes were obtained from Zen-Bio, Inc., and maintained in a 96-well plate in Adipocyte Maintenance Medium provided by Zen-Bio. For phosphor-ERK assays, cells were pretreated with Opti-MEM reduced serum medium (ThermoFisher, USA) supplemented with 0.1% FBS for 4 hours, and then treated with serially diluted ligands or antibodies. The level of ERK phosphorylation was determined using the AlphaScreen SureFire p-ERK 1/2 (Thr202/Tyr204) Assay Kit (Perkin Elmer, Waltham, Mass.) following the manufacturer's recommendations.

### 7.9.2. Results

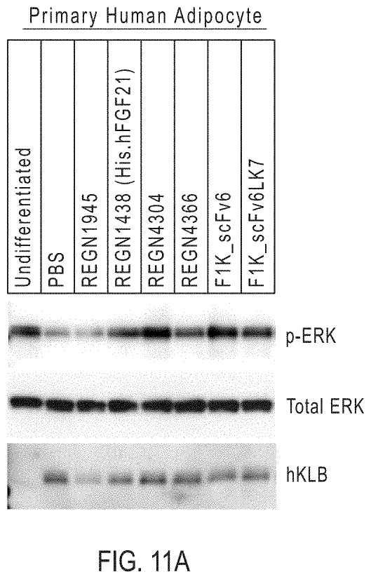

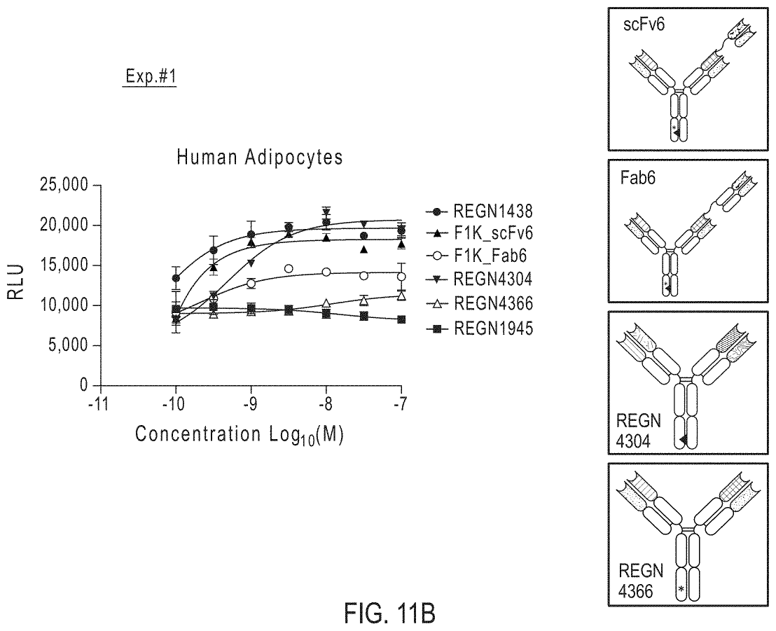

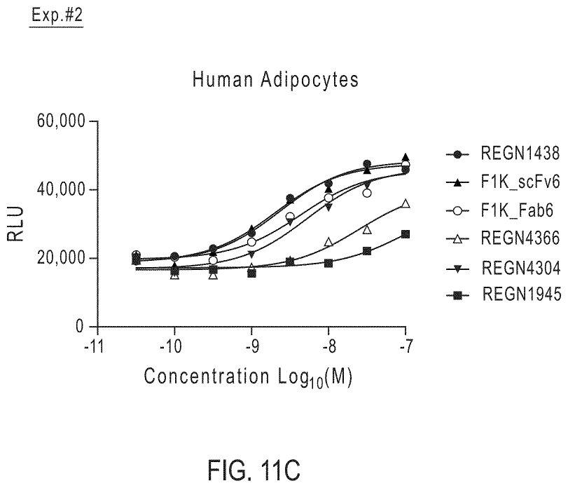

To determine the agonist activity of F1K_scFv6 and F1K_scFv6LK7 in human adipocytes that expressed FGFR1c and KLB endogenously, primary human adipocytes were treated with these molecules (FIG. 11A). KLB expression was induced during adipocyte differentiation. F1K_scFv6 and F1K_scFv6LK7 induced phospho-ERK in human adipocytes, which was comparable to REGN1438 (i.e., FGF21) treatment.

To determine the dose-dependent effects of F1K_scFv6 and F1K_Fab6 on FGFR1c signaling, human adipocytes were treated with serially diluted drugs, Phosho-ERK levels were measured using AlphaScreen SureFire p-ERK 1/2 (Thr202/Tyr204) Assay Kit (FIG. 11A). F1K_scFv6 and F1K_Fab6 strongly induced p-ERK with higher efficacy than the parental bispecific antibody (REGN4366) and REGN4304, indicating that F1K_scFv6 and F1K_Fab6 are potent agonists activating FGFR1c/KLB signaling.

### 7.10. Example 9: Size Analysis of In Vitro Complexes Formed Between KLB, FGFR1c and Binding Molecules by Asymmetric Flow Field-Flow Fractionation Coupled to Multi-Angle Laser Light Scattering (A4F-MALLS)

### 7.10.1. Overview

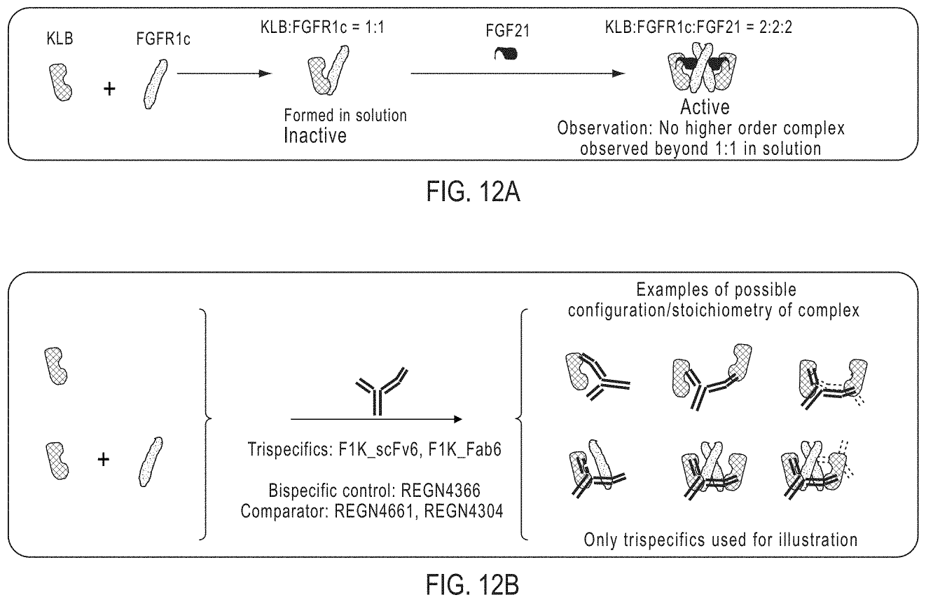

In principle, the trispecific binding molecules of the disclosure can form different types of complexes with FGFR1c and KLB as illustrated in FIG. 12A and FIG. 12B. To determine the types of complexes formed, size analysis of in vitro complexes formed between the 2+1 N-scFv and 2+1 N-Fab trispecific binding molecules was performed using asymmetric flow field-flow fractionation coupled to multi-angle light scattering (A4F-MALS). A4F-MALLS was also used to analyze complexes formed by a control bispecific binding molecule (REGN4304) and a monospecific KLB binding molecule (REGN4661).

### 7.10.2. Materials & Methods

### 7.10.2.1. A4F-MALLS Mobile Phase Buffer

The mobile phase buffer (10 mM sodium phosphate, 500 mM sodium chloride, pH 7.0±0.1) was prepared by combining 1.4 g sodium phosphate monobasic monohydrate, 10.7 g sodium phosphate dibasic heptahydrate, and 500 mL 5 M sodium chloride; the solution was then brought to a volume to 5.0 L with HPLC grade water. The final measured pH of the buffer was 7.0. The mobile phase buffer was filtered (0.2 μm) before use.

### 7.10.2.2. A4F-MALLS

The A4F-MALLS system was composed of an Eclipse™ 3+A4F Separation System coupled to an Agilent 1200 Series HPLC system equipped with an ultraviolet (UV) diode array detector, Wyatt Technology Dawn HELEOS® II laser light scattering instrument (LS), and an Optilab® T-rEX differential refractometer (RI) detector. The detectors were connected in series in the following order: UV-LS-RI. LS and RI detectors were calibrated according to instructions provided by Wyatt Technology.

Defined amounts of anti-KLB and anti-FGFR1c multispecific binding molecule candidates were each combined with REGN6424 (recombinant KLB) and REGN6152 (recombinant FGFR1c) and diluted in 1×DPBS, pH 7.4 to yield the equimolar ratio: 0.2 μM multispecific binding molecule:0.2 μM REGN REGN6424 or 0.2 μM multispecific binding molecule:0.2 μM REGN REGN6424: 0.2 μM REGN REGN6152. All samples were incubated at ambient temperature for 2 hours and maintained unfiltered at 4° C. prior to injection into an Eclipse™ short channel fitted with a W350 spacer foil (350 μm spacer thickness, 2.2 cm spacer width) and using a 10 kDa MWCO regenerated cellulose membrane. The channel was pre-equilibrated with the mobile phase buffer (10 mM sodium phosphate, 500 mM sodium chloride, pH 7.0±0.1), prior to the injection of each sample. Bovine serum albumin (BSA; 2 mg/mL; 10 μg sample load) was injected separately and included as a system suitability control.

The fractionation method consisted of four steps: injection, focusing, elution, and a channel “wash-out” step. The A4F-MALLS mobile phase buffer (10 mM sodium phosphate, 500 mM sodium chloride, pH 7.0±0.1) was used throughout the fractionation method. Each sample (7 μg) was injected at a flow rate of 0.2 mL/min for 1 min and subsequently focused for 3 min with a focus flow rate of 1.0 mL/min. The sample was eluted with a channel flow rate of 1.0 mL/min with the constant cross flow 3.0 mL/min for 15 min, followed by linear gradient cross flow from 3.0 mL/min to 0 mL/min over 5 min. Finally, the cross flow was held at 0 mL/min for an additional 5 min to wash out the channel. BSA was fractionated using the same parameter settings.

### 7.10.2.3. MALLS Data Analysis

Data were analyzed using ASTRA V software (version 5.3.4.14, Wyatt Technology). The data were fit to the equation that relates the excess scattered light to the solute concentration and weight-average molar mass, Mw (Kendrick et al., 2001, Anal Biochem. 299(2):136-46; Wyatt, 1993, Anal. Chim. Acta 272(1):1-40):

$$\frac{K ⋆ c}{R ( θ , c )} = \frac{1}{MwP ( θ )} + 2 {A}_{2} c \qquad Equation 1$$

where c is the solute concentration, R(θ,c) is the excess Raleigh ratio from the solute as a function of scattering angle and concentration, Mw is the molar mass, P(θ) describes the angular dependence of scattered light (˜1 for particles with radius of gyration <50 nm), A2 is the second virial coefficient in the expansion of osmotic pressure (which can be neglected since measurements are performed on dilute solutions) and

$${K}^{*} = \frac{4 {π}^{2} {n}_{0}^{2}}{{N}_{A} {λ}_{0}^{4}} {( \frac{dn}{dc} )}^{2} \qquad Equation 2$$

where n₀ represents the solvent refractive index, N_{A} is Avogadro's number, λ0 is the wavelength of the incident light in a vacuum, and dn/dc represents the specific refractive index increment for the solute.

The molar mass of BSA monomer served to evaluate the calibration constants of the light scattering and differential refractive index detectors during data collection (system suitability check). The relative standard deviation (% RSD) of the average molar mass of BSA determined from the UV and RI detectors was 5.0%.

The normalization coefficients for the light scattering detectors, inter-detector delay volume and band broadening terms were calculated from the BSA chromatograms collected for the A4F-MALLS condition employed. These values were applied to the data files collected for all the other samples to correct for these terms.

The dn/dc value and the extinction coefficient at 215 nm were experimentally determined using the protein conjugate analysis provided in the Astra software. The corrected extinction coefficient and dn/dc value was used to analyze all protein-protein complex samples.

### 7.10.3. Results

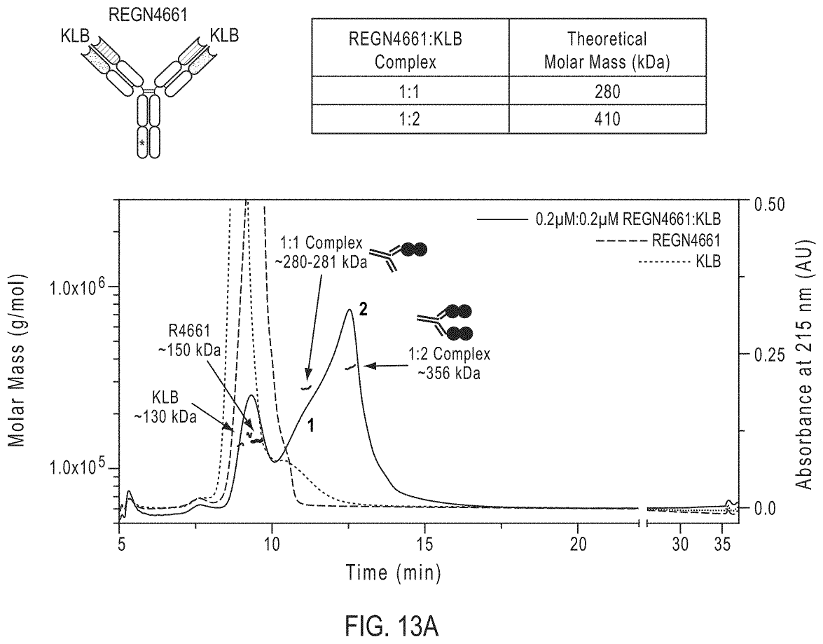

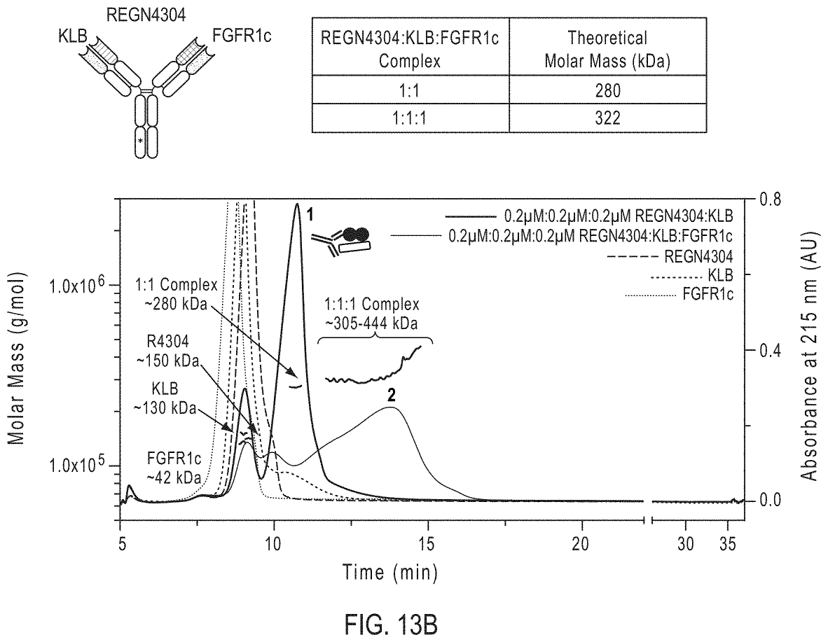

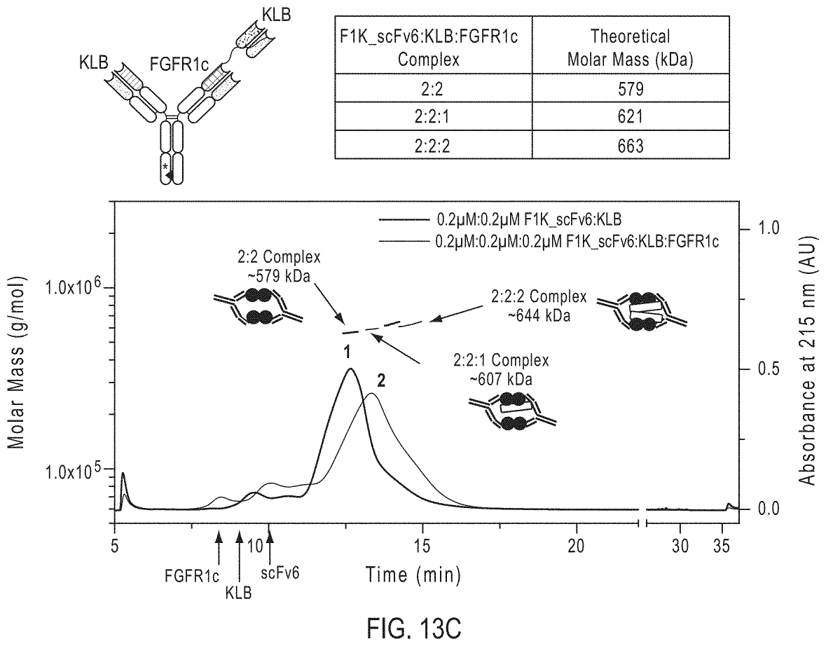

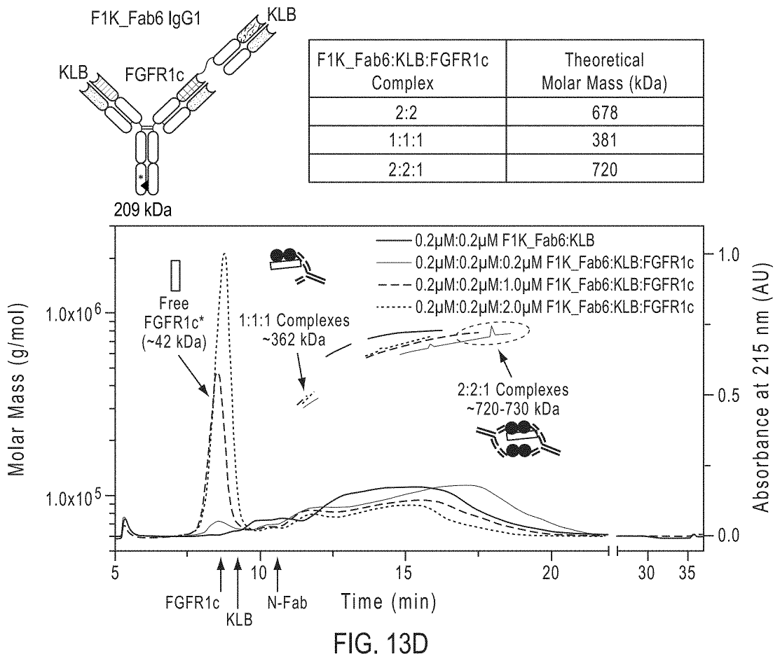

A4F-MALLS was used to assess the relative size distribution of complexes formed between recombinant KLB (REGN6424), recombinant FGFR1c (REGN6152), and several monospecific (REGN4661), bispecific (REGN4304), and trispecific (2+1 N-scFv and 2+1 N-Fab) binding molecules. The results are shown in FIG. 13A (for REGN4661), FIG. 13B (for 4304), FIG. 13C (the 2+1 N-scFv format) and FIG. 13D (the 2+1 N-Fab format). The theoretical molar mass and predicted stoichiometry of potential antibody:antigen complexes are provided as insets in FIGS. 13A-13D. As expected, the monospecific KLB binding molecule (REGN4661) formed canonical 1:1 (Peak 1, ˜280 kDa) and 1:2 (Peak 2, ˜356 kDa) complexes with KLB when combined at equimolar ratios (FIG. 13A). Similarly, when the control bispecific binding molecule (anti-KLB×FGFR1c; REGN4304) was mixed with an equimolar amount of KLB, a discrete, homogeneous peak (Peak 1) with a calculated molar mass of ˜280 kDa was observed (FIG. 13B). Based on the calculated molar mass of the individual components, peak 1 likely represents a 1:1 bispecific:KLB complex. Further addition of FGFR1c to this mixture resulted in a broad peak (Peak 2) with a calculated molar mass range of ˜305-444 kDa, which is generally consistent with a 1:1:1 bispecific:KLB:FGFR1c ternary complex (FIG. 13B). The upward trend in molar mass at the trailing end of peak 2 suggests larger complexes, weakly associated via KLB-FGFR1c interactions, may also be present in solution, but easily dissociate upon fractionation.

In comparison to the control monospecific and bispecific binding molecules, each novel trispecific binding molecule bound KLB and FGFR1c with a unique, higher order stoichiometry. When mixed with an equimolar amount of KLB, F1K-scFv6 IgG1 formed a largely discrete, homogeneous peak (Peak 1) having a molar mass of ˜579 kDa, likely representing a complex containing 2 molecules of F1K-scFv6 IgG1 bound to 2 molecules of KLB (2:2 complex; FIG. 13C). Upon addition of varying amounts of FGFR1c to this mixture, a slightly broader, later-eluting peak (Peak 2) was observed with a calculated molar mass range of −607-644 kDa. Peak 2 likely represents a mixture of ternary complexes containing 2 molecules of F1K-scFv6 IgG1, 2 molecules of KLB, and 1-2 molecules of FGFR1c (2:2:1 and 2:2:2 complexes; FIG. 13C). In contrast, F1K-Fab6 IgG appeared to form a broad, heterogenous mixture of 2:2 and 2:3 complexes (Peak 2; 681-811 kDa) with KLB alone, whereas subsequent addition of FGFR1c resulted in a shift in both elution and molar mass consistent with a 2:2:1 F1K-Fab6 IgG:KLB:FGFR1c ternary complex (Peak 3, -720-730 kDa; FIG. 13D). A minor peak (Peak 1; ˜362 kDa) consistent with a 1:1:1 F1K-Fab6 IgG:KLB:FGFR1c complex can also be observed in these samples. The broadness of the peaks representing complexes of F1K-Fab6 IgG, KLB, and FGFR1c may indicate that the resulting complexes adopt heterogeneous conformations and/or rapidly dissociate upon fractionation. Taken together, these data reveal that both trispecific binding molecules can bind KLB and FGFR1c to form ternary complexes having unique stoichiometries compared to control monospecific and bispecific binding molecules.

### 7.11. Materials & Methods for of Constant Domain Variants of Trispecific Antibodies (Examples 10 to 14)

### 7.11.1. Vector Construction for Constant Domain Variants

DNA fragments encoding anti-KLB GH1 Fab, anti-KLB GH2 Fab, anti-KLB GH2 scFv, and anti-FGFR1c Fab domains; various amino acid linkers; and various IgG hinge and Fc domains were synthesized by Integrated DNA Technologies, Inc. (San Diego, Calif.) or Geneart/Thermo Fisher Scientific (Regensburg, Germany)

Mammalian expression vectors for individual polypeptide chains were created by either NEBuilder HiFi DNA Assembly Kit (New England BioLabs Inc.) or restriction digest followed by ligation following standard molecular cloning protocols provided by New England BioLabs Inc. DNAs were transfected as a single plasmid or as a heavy and light chain pair, following the manufacturer's protocol. 50 ml of cell culture supernatant was harvested and processed for purification via HiTrap™ Protein G HP or MabSelect SuRe pcc columns (Cytiva).

Certain constructs were expressed in Expi293F™ cells by transient transfection (Thermo Fisher Scientific). Proteins in Expi293F supernatant were purified using the ProteinMaker system (Protein BioSolutions, Gaithersburg, Md.) with either HiTrap™ Protein G HP or MabSelect SuRe pcc columns (Cytiva). After single step elution, antibodies were neutralized, dialyzed into a final buffer of phosphate buffered saline (PBS) with 5% glycerol, aliquoted and stored at −80° C. For some constructs, an additional step of size-exclusion chromatography with HiPrep 26/60 Sephacryl S-200 column was used.

Other expression vectors were stably expressed in a Chinese hamster ovary (CHO) expression system.

### 7.11.2. Kinetics Measurement of Fc Receptor Binding by Biacore

Briefly, surface plasmon resonance (SPR) experiments were performed at 25° C. on a Biacore T200 instrument employing a carboxymethyl dextran-coated (CM-5) chip. A mouse monoclonal anti-penta-histidine antibody (GE Healthcare) was immobilized on the surface of the CM-5 sensor chip using standard amine-coupling chemistry. 140RU-376RU of His-tagged human, monkey or mouse FcγR proteins were captured on the anti-penta-histidine amine-coupled CM-5 chip and stock solutions of antibodies were injected at 50 μl/min for 2 min over the captured proteins and serially diluted (6 uM-24.7 nM). mAb binding response was monitored and, for low affinity receptors, steady-state binding equilibrium was calculated. Kinetic association (ka) and dissociation (kd) rate constants were determined by processing and fitting the data to a 1:1 binding model using Scrubber 2.0 curve fitting software. Binding dissociation equilibrium constants (KD) and dissociative half-lives (t1/2) were calculated from the kinetic rate constants as: KD (M)=kd/ka; and t1/2 (min)=(In2/(60*kd). Some KDs were derived using the steady state equilibrium dissociation constant; NB=no binding observed; IC=inconclusive affinity determination due to low specific RU signal.

### 7.11.3. Enzyme-Linked Immunosorbent Assay (ELISA)

Wells of microtiter plates were coated (18 h, 4° C.) with 4 μg/ml of 6×-His (SEQ ID NO:42) Tag monoclonal antibody (4E3D10H2/E3) (Thermo scientific) in 100 μl of PBS and were then blocked with blocking buffer (2% BSA in PBS) for 1 h at room temperature. Different Fc receptors (2 μg/ml, 100 μl/well) were loaded in duplicates and incubated for 1 h at room temperature. Meanwhile, the antibodies were diluted with a ratio of 1:5 from a starting concentration of 6.0×10⁻⁰⁶ M in blocking buffer. The diluted antibodies (100ul) were then added into the wells and incubated for 1 h at room temperature. Peroxidase-conjugated Goat Anti-Human IgG, F(ab′)₂ detection antibody 100ul/well (1:5000 in blocking buffer) was added for 1 h at room temperature and the reaction was visualized by the addition of 100 μl peroxidase substrate (KPL-TMB) for 30 min. The reaction was stopped with 100 μl TMB stop buffer and measured the absorbance at 450 nm using ELISA plate reader (Envision, PerkinElmer). Plates were washed three times with wash buffer (PBS, pH 7.4, containing 0.05% (v/v) Tween 20) after each step.

### 7.11.4. Surrogate ADCC Assay

### 7.11.4.1. Target Cells

HEK293/hFGFR1c/hKLB/hCD20: HEK293 cells where endogenous FGFR1 was excised by CRISPR-Cas9, were engineered to constitutively express full length human CD20 (hCD20, amino acids M1-P297 of accession number NP_690605.1), FGFR1c (hFGFR1c, amino acids M1-R731 of accession number NP_075594), and KLB (hKLB, amino acids M1-51044 of accession number NP_783864.1) Cells were sorted for high expression of all receptors.

### 7.11.4.2. Reporter Cells

Jurkat/NFAT-Luc/FcγR3a 176Val: Jurkat T cells were engineered to stably express a Nuclear Factor of Activated T-cells (NFAT) luciferase reporter construct along with the high affinity human FcγR3a 176Val allotype receptor (amino acids M1-K254 of accession number P08637 VAR_003960).

### 7.11.4.3. Assay Set-Up

Three days before the experiment, Jurkat reporter cells were split to 1.25×10⁵ cells/ml in RPM11640+10% FBS+P/S/G+0.5 μg/ml puromycin+500 μg/ml G418 growth media. On the day of the experiment, the target and reporter cells were transferred into assay media (RPMI+10% FBS+P/S/G) and added at a 1:1 ratio (3×10⁴/well of each cell type) to 96-well white microtiter plates. Multi-specific anti-FGFR1c/KLB antibodies and an hIgG4 S108P isotype control antibody were titrated in a 7-point, 1:4 serial dilution ranging from 73.2 μM to 300 nM final concentration, with the final 8th point containing no antibody, and added to the cells in duplicate. Plates were incubated at 37° C./5% CO2 for 4.6 h followed by the addition of an equal volume of ONE-Glo™ (Promega) reagent to lyse cells and detect luciferase activity. The emitted light was captured in Relative Light Units (RLU) on a multi-label plate reader Envision (Perkin Elmer). EC50 values of the antibodies were determined from a 4 parameter logistic equation over an 8-point dose response curve (including the background signal) using GraphPad Prism software. Maximum fold induction was calculated using the following equation:

Fold Induction=Max Average RLU within tested dose range of each antibody/Average RLU (background signal=no antibody)

### 7.11.5. Stable Expression and Antibody Titer

Recombinant proteins encoding different antibodies with various IgG subclasses were cloned into expression plasmids, transfected into CHO cells and stably transfected pools were isolated after selection with 400 mg/L hygromycin for 12-14 days. The stable CHO-cell pools, grown in chemically-defined protein-free medium in suspension, were used to produce proteins for testing.

Protein was produced by inducing cell cultures with 0.5 mg/L Doxycycline for five days and harvesting the conditioned media. Protein titers were determined with an Octet instrument (ForteBio) using a protein A sensor against a known standard at various concentrations

### 7.11.6. Luciferase Reporter Assay

Antibodies including different IgG hinge and Fc domains were tested for their agonist activities using HEK293.SREluc.hFGFR1c/hKLB cells that stably expressed human FGFR1c and KLB as well as a luciferase reporter gene under the control of a promoter containing serum responsive elements (SRE). Recombinant human FGF21 with 6×His (SEQ ID NO:42) tag was used as a positive control, with the maximum reporter activity obtained from FGF21 defined as 100% activity. Cells were treated with each antibody or 6×His-FGF21 for 6 hours, and then subjected to luciferase assays. The percent activity induced by individual antibodies was normalized against the maximum activity by FGF21. Dose-response assays were performed to determine EC50. The anti FelD1 isotype (hIgG4-5108P) control antibody, was used as a negative control.

### 7.11.7. Human Primary Adipocytes Signaling Assay

Human primary adipocytes differentiated from subcutaneous preadipocytes were obtained from Zen-Bio Inc (Durham, N.C.). Cells were cultured in a serum free media for 4 hours, and then treated with serially diluted antibodies for 15 minutes. Cells were lysed using a lysis buffer for AlphaScreen™ SureFire™ ERK Assay kit that measures phospho-ERK in the treated cell lysates (PerkinElmer, Shelton, Conn.). SureFire™ ERK Assay was performed according to the manufacturer's protocol. His-tagged human FGF21 and an isotype control human IgG4 antibody were tested as a positive and negative controls, respectively. An FGFR1c/KLB bispecific antibody was also included in the experiment.

### 7.12. Example 10: Design, Cloning, and Expression of IgG1 PVA Constant Domains

### 7.12.1. Overview

IgG1 Fc and IgG4 Fc have differing Fc gamma receptor binding capacity and charge distribution, which provide options for optimal Fc function engagement and varied compatibility with antibody building blocks such as Fabs, scFvs, and alternative format antibody fusion proteins. The hinge regions of IgG1 and IgG4 also have differing lengths and flexibility. IgG4 (S108P, or S228P, EU numbering) has been utilized in multiple approved antibody products, such as pembrolizumab, nivolumab and Ixekizumab, where reduced Fc effector function is needed. Due to the preference of antibody building blocks (e.g., Fabs, scFvs) for particular immunoglobulin subclasses, human IgG1 Fc-based alternative and natural sequence variant differing from IgG4 (S108P)—which demonstrates reduced Fc gamma receptor binding and reduced Fc receptor effector functions—was sought.

FIG. 17 presents an alignment of various IgG hinge/Fc variants with sequences between various wild type and modified human IgG1 and IgG4 hinge regions and a description of CH2 and CH3 Fc regions used, from amino acid 226 to 447 (EU numbering). hIgG1 PVA was designed to include the PVA mutation in the lower hinge region in an otherwise fully-IgG1 background (e.g., IgG1 upper hinge, CH2, and CH3 regions).

To test the properties of the hIgG1 PVA, it was incorporated into alternative format antibodies having either a 2+1 N-scFv or 2+1 N-Fab format (see, e.g., FIG. 5 for an illustration of the 2+1 N-scFv format; in the 2+1 N-Fab format, the N-terminal scFv domain is replaced by a Fab domain, as also shown in FIG. 5).

### 7.12.2. Results

Controls and bispecific antibodies incorporating the various IgG hinge and Fc domains were successfully expressed and purified.

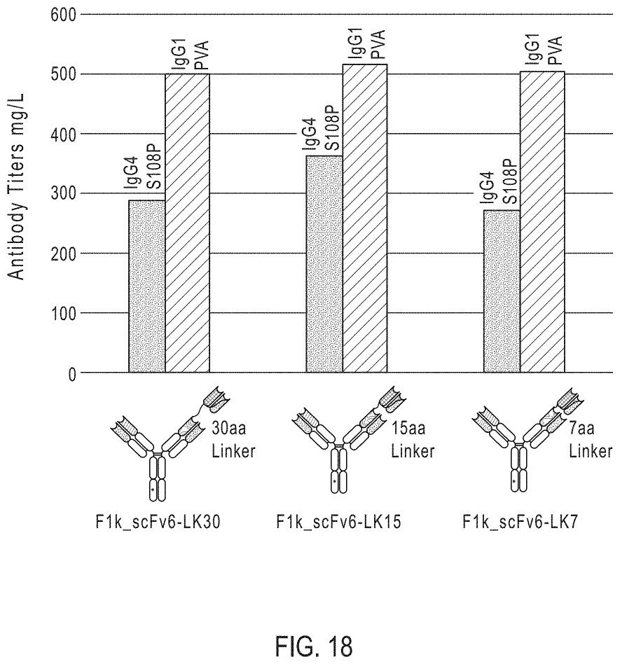

When expressed in CHO cells, F1K_scFv6 constructs in IgG1 PVA backbone with varied linker lengths between the scFv and the Fab had higher antibody titer (measured as the total antibody species) than constructs including IgG4 S108P (FIG. 18).

### 7.13. Example 11: Binding Kinetics of Constant Domain Variants for Fc Gamma Receptors

### 7.13.1. Overview

Binding affinities and signals of various antibodies with different hinge-Fc regions to Fc gamma receptors were measured by Biacore as described in Section 7.11.2.

### 7.13.1.1. Results

The results are shown in Tables 14 and 15 below.

TABLE 14
Binding signal of antibody with various Fc variants to human FcγR Binding

| | | FcR capture (RU) | | | | | | |
|---|---|---|---|---|---|---|---|---|
| | | | 164.4 ± 0.4 | 364.4 ± 0.6 | | 208 ± 1.7 | 899 ± 2.7 | |
| | | 615.9 ± 0.8 | Human | Human | 378.2 ± 1 | Human | Human | 548 ± 2.4 |
| | | Human | FcγRIIa | FcγRIIa | Human | FcγRIIIa | FcyRIIIa | Human |
| Antibody | Fc variant | FcγRI | (R131) | (H131) | FcγRIIb | (F176) | (V176) | FcγRIIIb |
| mAb1 | hIgG1 | 1606 | 457 | 614 | 833 | 322 | 2228 | 1028 |
| mAb2 | hIgG4 S108P | 1475 | 181 | 393 | 737 | 28 | 429 | −2 |
| mAb3 | hIgG4s | 36 | 340 | 419 | 226 | 6 | 34 | −8 |
| AF2a | hIgG1 PVA | 6 | 91 | 21 | −1 | 4 | 144 | −5 |
| AF2c | hIgG1 N180G | 518 | −5 | −9 | −9 | −5 | −11 | −7 |

TABLE 15
KD (M) Values for High and Low Affinity FcγR Binding

| | | | Human | Human | | Human | Human | |
|---|---|---|---|---|---|---|---|---|
| | | Human | FcγRIIa | FcγRIIa | Human | FcγRIIIa | FcyRIIIa | Human |
| Antibody | Fc variant | FcγRI | (R131) | (H131) | FcγRIIb | (F176) | (V176) | FcγRIIIb |
| mAb1 | hIgG1 | 1.76E−09 | 3.40E−07 | 3.40E−07 | 5.40E−07 | 1.02E−06 | 6.20E−07 | 1.02E−06 |
| mAb2 | hIgG4 S108P | 5.35E−09 | 5.70E−06 | 1.50E−06 | 7.20E−07 | WB | 4.90E−06 | NB |
| mAb3 | hIgG4s | NB | 1.32E−06 | 1.61E−06 | 1.23E−05 | NB | WB | NB |
| AF2a | hIgG1 PVA | NB | WB | WB | NB | NB | 7.20E−05 | NB |
| AF2c | hIgG1 N180G | 2.21E−06 | NB | NB | NB | NB | NB | NB |

In Table 15, NB refers to No Binding; WB refers to Weak Binding

IgG1 PVA has no binding signal in FcγR1, FcγR2b, FcγR3a (F176), FcγR3b. It has low binding signal to FcγR2a (both R131 and H131) but at a significantly reduced level (91 and 21 RU respectively) in comparison to IgG1 and IgG4 S108P. IgG1 PVA has weak to medium binding signal (144 RU) to FcγR3a (V176) with a KD=7.2×10⁻⁰⁵ M, much weaker than that for IgG1 and IgG4 S108P (Table 14 and Table 15).

### 7.14. Example 12: ELISA Binding to Fc Gamma Receptors

### 7.14.1. Overview

Binding of FGFR1c/KLB trispecific antibodies including various IgG hinge and Fc regions was assessed by ELISA as described in Section 7.11.3.

### 7.14.2. Results

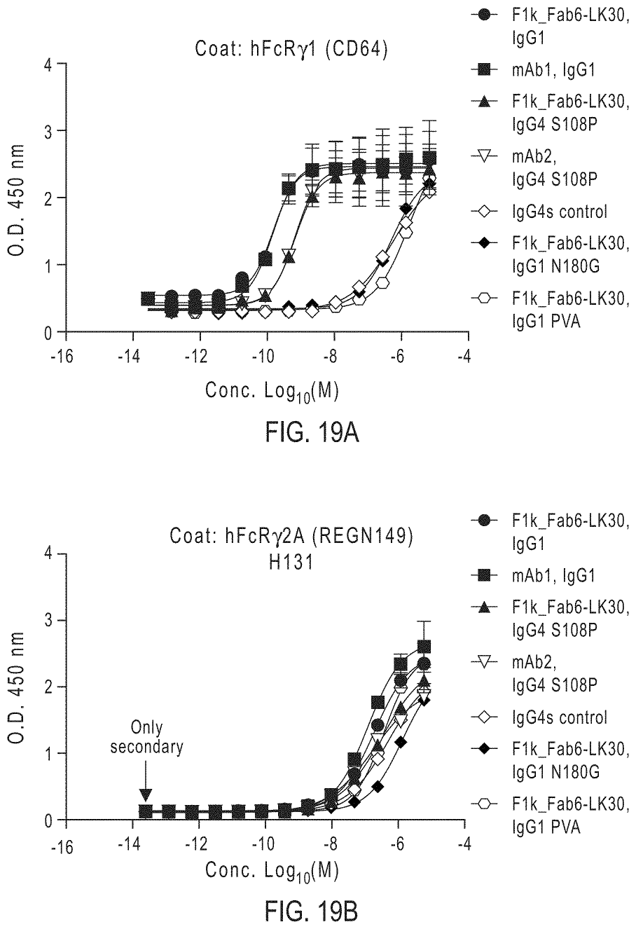

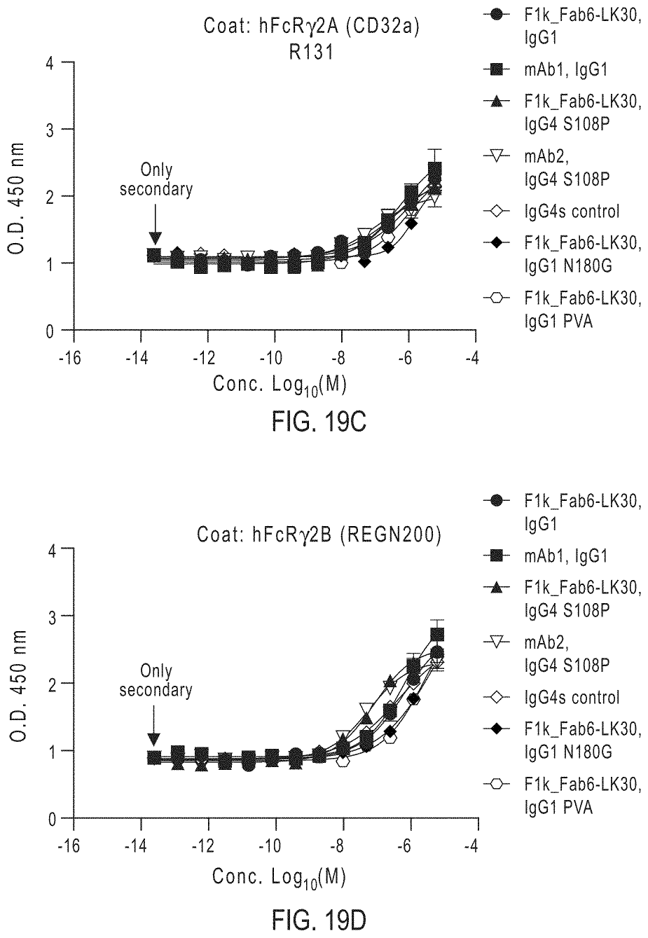

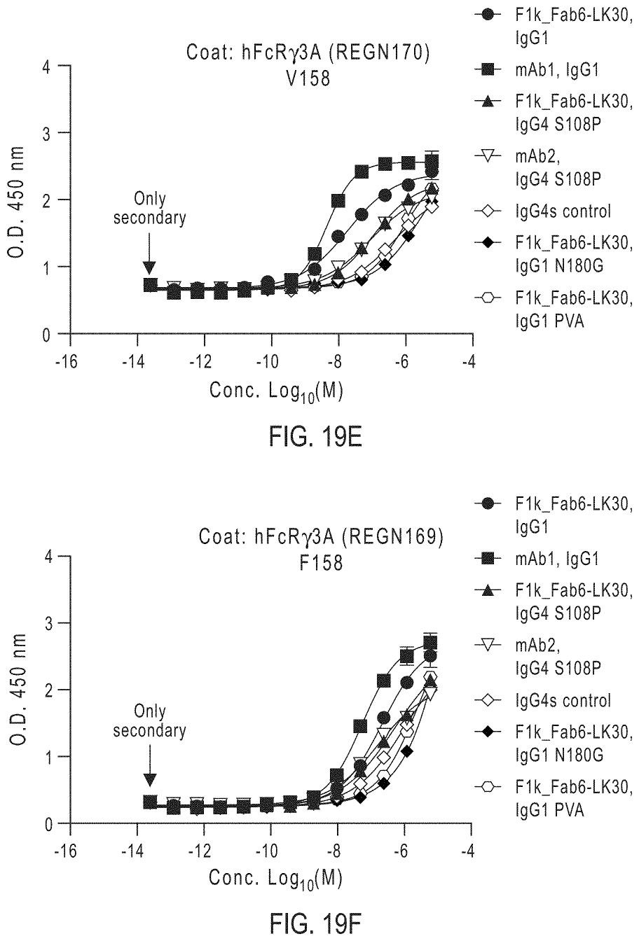

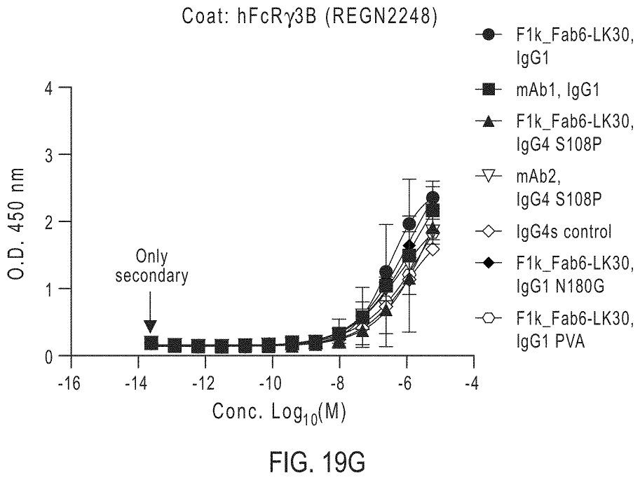

Binding curves indicating the ability of the controls and test antibodies to bind various Fc gamma receptors are depicted in FIGS. 19A-19G. Antibodies harboring the wild type IgG1 hinge and Fc domain demonstrated the highest binding to hFCRγ1. Binding of hFCRγ1 was significantly reduced with IgG1 PVA, showing similar binding to IgG4s (FIG. 19A). Binding with IgG1 N180G was similarly reduced. IgG4 S108P demonstrated only slightly reduced binding to hFCRγ1 relative to wild type IgG1. A similar trend was observed in the binding of hFCRγ3A (V158) and hFCRγ3A (F158) (FIG. 19E, FIG. 19F). Very little difference in binding was observed with hFCRγ2A (H131), hFCRγ2A (R131) (FIG. 19B and FIG. 19C). But IgG1 PVA has weaker binding than IgG4 S108P and slightly weaker than IgG1 in hFCRγ2B (FIG. 19D). In hFCRγ3B, IgG1 PVA has less binding than IgG1 (FIG. 19G).

### 7.15. Example 13: Antibody-Dependent Cellular Cytotoxicity

### 7.15.1. Overview

Utilizing the surrogate antibody-dependent cellular cytotoxicity (ADCC) assay described in Section 7.11.4, cytotoxic activity of IgG1 PVA was determined and compared to the cytotoxic activity of other IgG variants (e.g., IgG1 N180G and IgG4 S108P).

The ability of trispecific antibodies targeting hFGFR1c and hKLB to interact with FcγR3a, an Fc-receptor prominently expressed on NK cells that induces antibody dependent cell-mediated cytotoxicity (ADCC), was measured in a surrogate bioassay using reporter cells and target cells bound to antibodies. In this assay, engineered Jurkat T cells express the reporter gene luciferase under the control of the transcription factor NFAT (NFAT-Luc) along with the high affinity human FcγR3a 176Val allotype receptor (Jurkat/NFAT-Luc/hFcγR3a ¹⁷⁶Val). Target cells are HEK293 cells engineered to express human CD20 in combination with full length human FGFR1c and human KLB. Reporter cells are incubated with target cells and engagement of FcγR3a via the Fc domain of human IgG1 antibodies bound to target cells leads to the activation of the transcription factor NFAT in the reporter cells and drives the expression of luciferase which is then measured via a luminescence readout.

### 7.15.2. Results

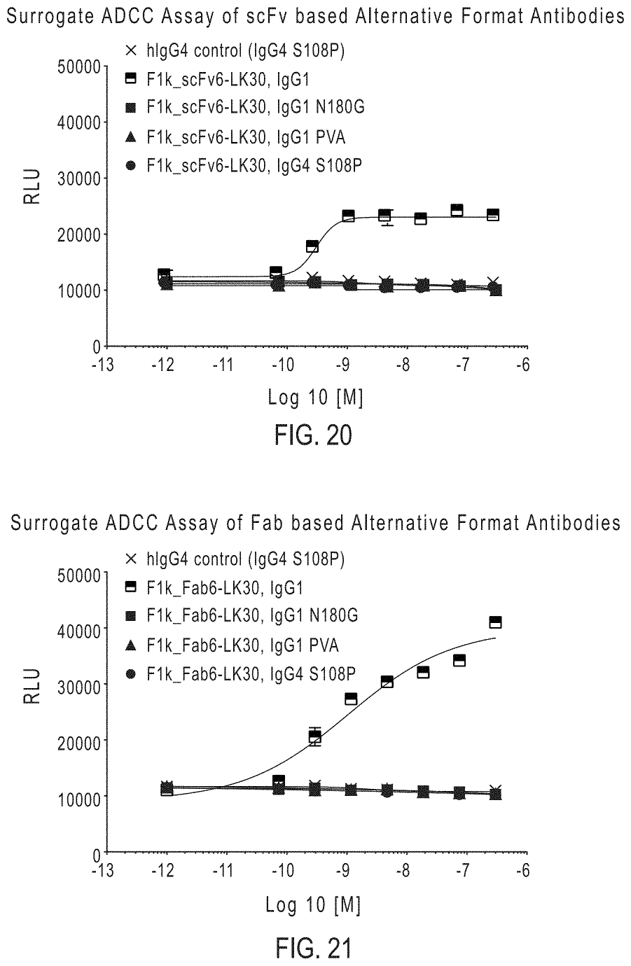

Representative data from the ADCC assays are depicted in FIGS. 20 and 21. Only antibodies with wild type IgG1, F1K_scFv6-LK30 IgG1 and F1K_Fab6-LK30 IgG1, showed induction of luciferase signal, by 1.9 fold (EC50=307 μM) and 3.4 fold (EC50=1.04 nM), respectively. None of the trispecific antibodies in 2+1 N-scFv or 2+1 N-Fab formats with IgG1 PVA, IgG1 N180G or IgG4 S108P showed activity in the surrogate ADCC assay.

### 7.16. Example 14: Molecule Activity

### 7.16.1. Overview

The activity of FGFR1c/KLB trispecific antibodies including IgG1 PVA and controls was tested utilizing the luciferase reporter assay and human primary adipocyte signaling assay described in Sections 7.11.6 and 7.11.7.

### 7.16.2. Results

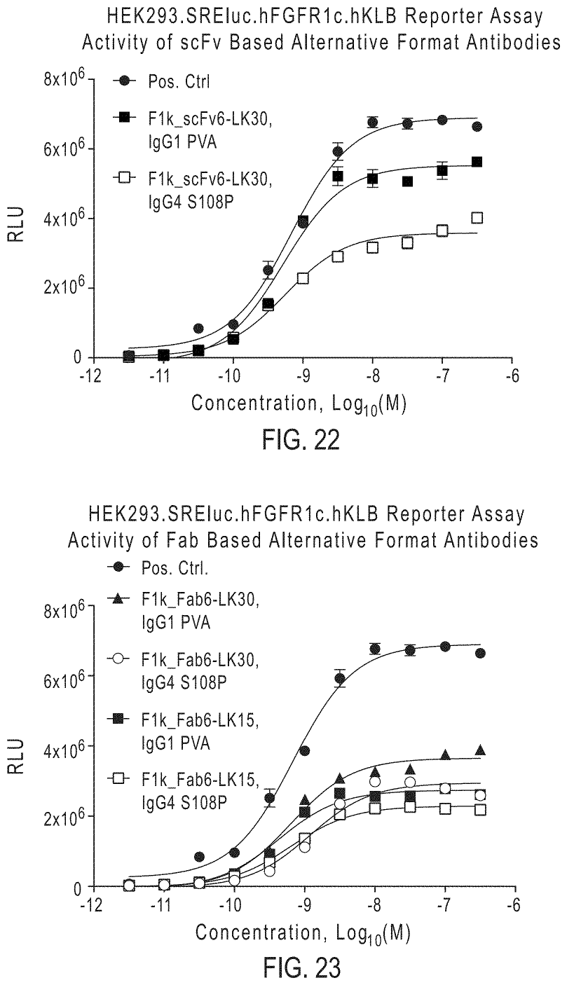

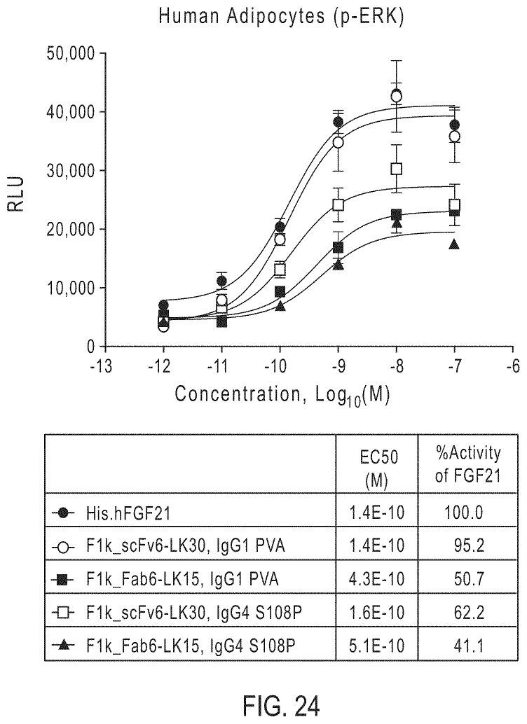

Activity of trispecific antibodies in HEK.293SREluc.hFGFR1c/hKLB is shown in FIG. 22 (F1K_scFv6-LK30, IgG1 PVA and F1K_scFv6-LK30, IgG4 S108P) and FIG. 22 (F1K_Fab6-LK30, IgG1 PVA; F1K_Fab6-LK15, IgG1 PVA; F1K_Fab6-LK30, IgG4 S108P and F1K_Fab6-LK15, IgG4 S108P). Activity in human adipocytes is shown in FIG. 24 (F1K_scFv6-LK30, IgG1 PVA; F1K_scFv6-LK30, IgG4 S108P; F1K_Fab6-LK15, IgG1 PVA; and F1K_Fab6-LK15, IgG4 S108P). The antibody with 2+1 N-scFv format incorporating IgG1 PVA showed superior agonist activity to that with IgG4 S108P in both HEK FGFR1c/KLB cells (FIG. 22) and human adipocytes (FIG. 23). The antibody with the 2+1 N-Fab format with an IgG1 PVA constant domain caused greater maximum activation than with an IgG4 S108P constant domain in the reporter cell assay (FIG. 24).

### 8. SPECIFIC EMBODIMENTS

The present disclosure is exemplified by the specific embodiments below.

1. A method comprising administering a multispecific binding molecule (MBM) or a pharmaceutical composition comprising the MBM to a subject, wherein the MBM comprises:
(a) an antigen-binding module 1 (ABM1) that specifically binds to human fibroblast growth factor receptor 1c isoform (“FGFR1c”);
(b) an antigen-binding module 2 (ABM2) that specifically binds to the GH1 domain of human klotho beta (“KLB”); and
(c) an antigen-binding module 3 (ABM3) that specifically binds to the GH2 domain of human KLB.

2. The method of embodiment 1, wherein the MBM is administered to the subject in an amount effective to:
(a) treat a metabolic condition; and/or
(b) improve metabolism.

3. The method of embodiment 1 or embodiment 2, wherein the method is effective to agonize FGF21 receptor complexes in the subject.

4. The method of any one of embodiments 1 to 3, wherein each antigen-binding module is capable of binding its respective target at the same time as each of the other antigen-binding modules is bound to its respective target.

5. The method of any one of embodiments 1 to 4, wherein ABM1 binds to loop D3 of FGFR1c.

6. The method of any one of embodiments 1 to 4, wherein ABM1 binds to loop D2 of FGFR1c.

7. The method of any one of embodiments 1 to 6, wherein the MBM is a trispecific binding molecule (“TBM”).

8. The method of any one of embodiments 1 to 7, wherein ABM1 is an antibody fragment, an scFv, a dsFv, a Fv, a Fab, an scFab, a (Fab′)2, a single domain antibody (SDAB), a VH or VL domain, or a camelid VHH domain.

9. The method of one any of embodiments 1 to 8, wherein ABM2 is an antibody fragment, an scFv, a dsFv, a Fv, a Fab, an scFab, a (Fab′)2, a single domain antibody (SDAB), a VH or VL domain, or a camelid VHH domain.

10. The method of any one of embodiments 1 to 9, wherein ABM3 is an antibody fragment, an scFv, a dsFv, a Fv, a Fab, an scFab, a (Fab′)2, a single domain antibody (SDAB), a VH or VL domain, or a camelid VHH domain.

11. The method of any one of embodiments 1 to 10, wherein ABM1 is an scFv.

12. The method of any one of embodiments 1 to 10, wherein ABM1 is a Fab.

13. The method of embodiment 12, in which a light chain of ABM1 is a universal light chain.

14. The method of embodiment 12, in which a light chain constant region and a first heavy chain constant region (CH1) of ABM1 are in a Crossmab arrangement.

15. The method of any one of embodiments 1 to 14, wherein ABM2 is an scFv.

16. The method of any one of embodiments 1 to 12, wherein ABM2 is a Fab.

17. The method of embodiment 16, in which a light chain of ABM2 is a universal light chain.

18. The method of embodiment 16, in which a light chain constant region and a first heavy chain constant region (CH1) of ABM2 are in a Crossmab arrangement.

19. The method of any one of embodiments 1 to 18, wherein ABM3 is an scFv.

20. The method of any one of embodiments 1 to 18, wherein ABM3 is a Fab.

21. The method of embodiment 20, in which a light chain of ABM3 is a universal light chain.

22. The method of embodiment 20, in which a light chain constant region and a first heavy chain constant region (CH1) of ABM3 are in a Crossmab arrangement.

23. The method of any one of embodiments 1 to 22, wherein the MBM comprises an Fc heterodimer.

24. The method of embodiment 23, wherein Fc domains in the Fc heterodimer comprise knob-in-hole mutations as compared to a wild type Fc domain.

25. The method of embodiment 23 or embodiment 24, wherein Fc domains in the Fc heterodimer comprise star mutations as compared to a wild type Fc domain.

26. The method of any one of embodiments 23 to 25, wherein the MBM comprises:
(a) a first polypeptide chain comprising, in an N- to C-terminal orientation, (i) an scFv operably linked to (ii) a first heavy chain region of a first Fab operably linked to (iii) an Fc domain;
(b) a second polypeptide chain comprising, in an N- to C-terminal orientation, (i) a second heavy chain region of a second Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.

27. The method of embodiment 26, in which the first light chain and the second light chain are identical.

28. The method of embodiment 26 or embodiment 27, in which ABM1 is the first

Fab.

29. The method of embodiment 28, in which ABM2 is the scFv and ABM3 is the second Fab.

30. The method of embodiment 28, in which ABM2 is the second Fab and ABM3 is the scFv.

31. The method of any one of embodiments 26 to 30, in which the scFv is linked to the first heavy chain region via a linker.

32. The method of embodiment 31, wherein the linker is:
(a) at least 5 amino acids, at least 6 amino acids or at least 7 amino acids in length; and optionally

(b) up to 30 amino acids, up to 40 amino acids, up to 50 amino acids or up to 60 amino acids in length.

33. The method of embodiment 32, wherein the linker is:
(a) 5 amino acids to 50 amino acids in length;
(b) 5 amino acids to 45 amino acids in length;
(c) 5 amino acids to 40 amino acids in length;
(d) 5 amino acids to 35 amino acids in length;
(e) 5 amino acids to 30 amino acids in length;
(f) 5 amino acids to 25 amino acids in length; or
(g) 5 amino acids to 20 amino acids in length.

34. The method of embodiment 32, wherein the linker is:
(a) 6 amino acids to 50 amino acids in length;
(b) 6 amino acids to 45 amino acids in length;
(c) 6 amino acids to 40 amino acids in length;
(d) 6 amino acids to 35 amino acids in length;
(e) 6 amino acids to 30 amino acids in length;
(f) 6 amino acids to 25 amino acids in length; or
(g) 6 amino acids to 20 amino acids in length.

35. The method of embodiment 32, wherein the linker is:
(a) 7 amino acids to 40 amino acids in length;
(b) 7 amino acids to 35 amino acids in length;
(c) 7 amino acids to 30 amino acids in length;
(d) 7 amino acids to 25 amino acids in length;
(e) 7 amino acids to 20 amino acids in length.

36. The method of embodiment 32, wherein the linker is 5 amino acids to 45 amino acids in length.

37. The method of embodiment 32, wherein the linker is 7 amino acids to 30 amino acids in length.

38. The method of embodiment 32, wherein the linker is wherein the linker is 5 amino acids to 25 amino acids in length.

39. The method of embodiment 32, wherein the linker is 10 amino acids to 60 amino acids in length.

40. The method of embodiment 39, wherein the linker is 20 amino acids to 50 amino acids in length.

41. The method of embodiment 40, wherein the linker is 25 amino acids to 35 amino acids in length.

42. The method of any one of embodiments 31 to 41, wherein the linker is or comprises a multimer of GₙS (SEQ ID NO: 15) or SGₙ (SEQ ID NO: 16), where n is an integer from 1 to 7.

43. The method of embodiment 42, wherein the linker is or comprises a multimer of G45 (SEQ ID NO: 17).

44. The method of any one of embodiments 31 to 41, wherein the linker is or comprises a comprises two consecutive glycines (2Gly), three consecutive glycines (3Gly), four consecutive glycines (4Gly (SEQ ID NO: 18)), five consecutive glycines (5Gly (SEQ ID NO: 19)), six consecutive glycines (6Gly (SEQ ID NO: 20)), seven consecutive glycines (7Gly (SEQ ID NO: 21)), eight consecutive glycines (8Gly (SEQ ID NO: 22)) or nine consecutive glycines (9Gly (SEQ ID NO: 23)).

45. The method of any one of embodiments 23 to 25, wherein the MBM comprises:
(a) a first polypeptide chain comprising, in an N- to C-terminal orientation, (i) a first heavy chain region of a first Fab operably linked to (ii) a second heavy chain region of a second Fab operably linked to (iii) an Fc domain, optionally wherein optionally the first heavy chain region is linked to the second heavy chain regions via a linker, optionally wherein the linker is as defined in any one of embodiments 32 to 44;
(b) a second polypeptide chain comprising, in an N- to C-terminal orientation, (i) a third heavy chain region of a third Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab; and
(e) a fifth polypeptide chain comprising a third light chain that pairs with the third heavy chain region to form the third Fab.

46. The method of embodiment 45, in which the first, second and third Fabs are the only antigen binding modules.

47. The method of any one of embodiments 45 to 46, in which the first light chain and the second light chain are identical.

48. The method of any one of embodiments 45 to 47, in which ABM1 is the second Fab.

49. The method of embodiment 48, in which ABM2 is the first Fab and ABM3 is the third Fab.

50. The method of embodiment 48, in which ABM3 is the first Fab and ABM2 is the third Fab.

51. The method of any one of embodiments 1 to 50, in which ABM1 comprises CDR sequences set forth in Table 1B.

52. The method of any one of embodiments 1 to 51, in which ABM2 comprises CDR sequences set forth in Table 2B.

53. The method of any one of embodiments 1 to 52, in which ABM3 comprises CDR sequences set forth in Table 3B.

54. The method of any one of embodiments 1 to 53, wherein the MBM is a trivalent MBM.

55. The method of any one of embodiments 1 to 53, wherein the MBM is a tetravalent MBM.

56. The method of any one of embodiments 1 to 55, wherein the method is effective to reduce the weight of the subject.

57. The method of embodiment 1 to 56, wherein the method is effective to reduce circulating high-density lipoprotein cholesterol in the subject.

58. The method of embodiment 1 to 57, wherein the method is effective to increase circulating low-density lipoprotein cholesterol in the subject.

59. The method of embodiment 1 to 58, wherein the method is effective to reduce blood triglycerides in the subject.

60. The method of embodiment 1 to 59, wherein the method is effective to reduce blood glucose in the subject.

61. The method of embodiment 1 to 60, wherein the subject has a metabolic disorder.

62. The method of embodiment 61, wherein the metabolic disorder is metabolic syndrome.

63. The method of embodiment 61, wherein the metabolic disorder is obesity.

64. The method of embodiment 61, wherein the metabolic disorder is fatty liver.

65. The method of embodiment 61, wherein the metabolic disorder is hyperinsulinemia.

66. The method of embodiment 61, wherein the metabolic disorder is type 2 diabetes.

67. The method of embodiment 61, wherein the metabolic disorder is nonalcoholic steatohepatitis (“NASH”).

68. The method of embodiment 61, wherein the metabolic disorder is nonalcoholic fatty liver disease (“NAFLD”).

69. The method of embodiment 61, wherein the metabolic disorder is hypercholesterolemia.

70. The method of embodiment 61, wherein the metabolic disorder is hyperglycemia.

71. A method comprising administering a multispecific binding molecule (MBM) or a pharmaceutical composition comprising the MBM to a subject, wherein the MBM comprises:
(a) a first antigen-binding means for specifically binding to human fibroblast growth factor receptor 1c isoform (“FGFR1c”);
(b) a second antigen-binding means for specifically binding to the GH1 domain of human klotho beta (“KLB”); and
(c) a third antigen-binding means for specifically binding to the GH2 domain of human KLB.

72. The method of embodiment 71, wherein the MBM is administered to the subject in an amount effective to:
(a) treat a metabolic condition; and/or
(b) improve metabolism.

73. The method of embodiment 71 or embodiment 72, wherein the method is effective to agonize FGF21 receptor complexes in the subject.

74. The method of any one of embodiments 71 to 73, wherein each antigen-binding means is capable of binding its respective target at the same time as each of the other antigen-binding means is bound to its respective target.

75. The method of any one of embodiments 71 to 74, wherein the first antigen-binding means binds to loop D3 of FGFR1c.

76. The method of any one of embodiments 71 to 74, wherein the first antigen-binding means binds to loop D2 of FGFR1c.

77. The method of any one of embodiments 71 to 76, wherein the MBM is a trispecific binding molecule (“TBM”).

78. The method of any one of embodiments 71 to 77, wherein the first antigen-binding means is an antibody fragment, an scFv, a dsFv, a Fv, a Fab, an scFab, a (Fab′)2, a single domain antibody (SDAB), a VH or VL domain, or a camelid VHH domain.

79. The method of one any of embodiments 71 to 78, wherein the second antigen-binding means is an antibody fragment, an scFv, a dsFv, a Fv, a Fab, an scFab, a (Fab′)2, a single domain antibody (SDAB), a VH or VL domain, or a camelid VHH domain.

80. The method of any one of embodiments 71 to 79, wherein the third antigen-binding means is an antibody fragment, an scFv, a dsFv, a Fv, a Fab, an scFab, a (Fab′)2, a single domain antibody (SDAB), a VH or VL domain, or a camelid VHH domain.

81. The method of any one of embodiments 71 to 80, wherein the first antigen-binding means is an scFv.

82. The method of any one of embodiments 71 to 80, wherein the first antigen-binding means is a Fab.

83. The method of embodiment 82, in which a light chain of the first antigen-binding means is a universal light chain.

84. The method of embodiment 82, in which a light chain constant region and a first heavy chain constant region (CH1) of the first antigen-binding means are in a Crossmab arrangement.

85. The method of any one of embodiments 71 to 84, wherein the second antigen-binding means is an scFv.

86. The method of any one of embodiments 71 to 82, wherein the second antigen-binding means is a Fab.

87. The method of embodiment 86, in which a light chain of the second antigen-binding means is a universal light chain.

88. The method of embodiment 86, in which a light chain constant region and a first heavy chain constant region (CH1) of the second antigen-binding means are in a Crossmab arrangement.

89. The method of any one of embodiments 71 to 88, wherein the third antigen-binding means is an scFv.

90. The method of any one of embodiments 71 to 88, wherein the third antigen-binding means is a Fab.

91. The method of embodiment 90, in which a light chain of the third antigen-binding means is a universal light chain.

92. The method of embodiment 90, in which a light chain constant region and a first heavy chain constant region (CH1) of the third antigen-binding means are in a Crossmab arrangement.

93. The method of any one of embodiments 71 to 92, wherein the MBM comprises an Fc heterodimer.

94. The method of embodiment 93 or embodiment 94, wherein Fc domains in the Fc heterodimer comprise knob-in-hole mutations as compared to a wild type Fc domain.

95. The method of embodiment 93, wherein Fc domains in the Fc heterodimer comprise star mutations as compared to a wild type Fc domain.

96. The method of any one of embodiments 93 to 95, wherein the MBM comprises:
(a) a first polypeptide chain comprising, in an N- to C-terminal orientation, (i) an scFv operably linked to (ii) a first heavy chain region of a first Fab operably linked to (iii) an Fc domain;
(b) a second polypeptide chain comprising, in an N- to C-terminal orientation, (i) a second heavy chain region of a second Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.

97. The method of embodiment 96, in which the first light chain and the second light chain are identical.

98. The method of embodiment 96 or embodiment 97, in which the first antigen-binding means is the first Fab.

99. The method of embodiment 98, in which the second antigen-binding means is the scFv and the second antigen-binding means is the second Fab.

100. The method of embodiment 98, in which the second antigen-binding means is the second Fab and the third antigen-binding means is the scFv.

101. The method of any one of embodiments 96 to 100, in which the scFv is linked to the first heavy chain region via a linker.

102. The method of embodiment 101, wherein the linker is:
(a) at least 5 amino acids, at least 6 amino acids or at least 7 amino acids in length; and optionally
(b) up to 30 amino acids, up to 40 amino acids, up to 50 amino acids or up to 60 amino acids in length.

103. The method of embodiment 102, wherein the linker is:
(a) 5 amino acids to 50 amino acids in length;
(b) 5 amino acids to 45 amino acids in length;
(c) 5 amino acids to 40 amino acids in length;
(d) 5 amino acids to 35 amino acids in length;
(e) 5 amino acids to 30 amino acids in length;
(f) 5 amino acids to 25 amino acids in length; or
(g) 5 amino acids to 20 amino acids in length.

104. The method of embodiment 102, wherein the linker is:
(a) 6 amino acids to 50 amino acids in length;
(b) 6 amino acids to 45 amino acids in length;
(c) 6 amino acids to 40 amino acids in length;
(d) 6 amino acids to 35 amino acids in length;
(e) 6 amino acids to 30 amino acids in length;
(f) 6 amino acids to 25 amino acids in length; or
(g) 6 amino acids to 20 amino acids in length.

105. The method of embodiment 102, wherein the linker is:
(a) 7 amino acids to 40 amino acids in length;
(b) 7 amino acids to 35 amino acids in length;
(c) 7 amino acids to 30 amino acids in length;
(d) 7 amino acids to 25 amino acids in length;
(e) 7 amino acids to 20 amino acids in length.

106. The method of embodiment 102, wherein the linker is 5 amino acids to 45 amino acids in length.

107. The method of embodiment 102, wherein the linker is 7 amino acids to 30 amino acids in length.

108. The method of embodiment 102, wherein the linker is 5 amino acids to 25 amino acids in length.

109. The method of embodiment 102, wherein the linker is 10 amino acids to 60 amino acids in length.

110. The method of embodiment 109, wherein the linker is 20 amino acids to 50 amino acids in length.

111. The method of embodiment 110, wherein the linker is 25 amino acids to 35 amino acids in length.

112. The method of any one of embodiments 101 to 111, wherein the linker is or comprises a multimer of GₙS (SEQ ID NO: 15) or SGₙ (SEQ ID NO: 16), where n is an integer from 1 to 7.

113. The method of embodiment 109, wherein the linker is or comprises a multimer of G₄S (SEQ ID NO: 17).

114. The method of any one of embodiments 101 to 111, wherein the linker is or comprises a comprises two consecutive glycines (2Gly), three consecutive glycines (3Gly), four consecutive glycines (4Gly (SEQ ID NO: 18)), five consecutive glycines (5Gly (SEQ ID NO: 19)), six consecutive glycines (6Gly (SEQ ID NO: 20)), seven consecutive glycines (7Gly (SEQ ID NO: 21)), eight consecutive glycines (8Gly (SEQ ID NO: 22)) or nine consecutive glycines (9Gly (SEQ ID NO: 23)).

115. The method of any one of embodiments 93 to 95, wherein the MBM comprises:
(a) a first polypeptide chain comprising, in an N- to C-terminal orientation, (i) a first heavy chain region of a first Fab operably linked to (ii) a second heavy chain region of a second Fab operably linked to (iii) an Fc domain, optionally wherein optionally the first heavy chain region is linked to the second heavy chain regions via a linker, optionally wherein the linker is as defined in any one of embodiments 32 to 44;
(b) a second polypeptide chain comprising, in an N- to C-terminal orientation, (i) a third heavy chain region of a third Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab; and
(e) a fifth polypeptide chain comprising a third light chain that pairs with the third heavy chain region to form the third Fab.

116. The method of embodiment 115, in which the first, second and third Fabs are the only antigen binding modules.

117. The method of any one of embodiments 115 to 116, in which the first light chain and the second light chain are identical.

118. The method of any one of embodiments 115 to 117, in which the first antigen-binding means is the second Fab.

119. The method of embodiment 118, in which the second antigen-binding means is the first Fab and the third antigen-binding means is the third Fab.

120. The method of embodiment 118, in which the third antigen-binding means is the first Fab and the second antigen-binding means is the third Fab.

121. The method of any one of embodiments 71 to 120, in which the first antigen-binding means comprises CDR sequences set forth in Table 1B.

122. The method of any one of embodiments 71 to 121, in which the second antigen-binding means comprises CDR sequences set forth in Table 2B.

123. The method of any one of embodiments 71 to 122, in which the third antigen-binding means comprises CDR sequences set forth in Table 3B.

124. The method of any one of embodiments 71 to 123, wherein the MBM is a trivalent MBM.

125. The method of any one of embodiments 71 to 123, wherein the MBM is a tetravalent MBM.

126. The method of any one of embodiments 1 to 125, wherein the MBM comprises a heterodimeric pair of constant domains.

127. The method of embodiment 126, wherein each constant domain comprises one or more substitutions at S228, E233, L234, L235, D265, N297, P329 or P331 (all according to EU numbering).

128. The method of embodiment 127, wherein the constant domain comprises a S228P substitution.

129. The method of embodiment 127, wherein the constant domain comprises an E233A or E233P substitution.

130. The method of embodiment 127, wherein the constant domain comprises an L234A substitution.

131. The method of embodiment 127, wherein the constant domain comprises an L235A.

132. The method of embodiment 127, wherein the constant domain comprises a D265A substitution.

133. The method of embodiment 127, wherein the constant domain comprises an N297A or N297D substitution.

134. The method of embodiment 127, wherein the constant domain comprises a P329G or P329A substitution.

135. The method of embodiment 127, wherein the constant domain comprises a P331S.

136. The method of any one of embodiments 126 to 135, which comprises any combination of substitutions set forth in Section 6.2.7.1.

137. The method of any one of embodiments 126 to 136, wherein each constant domain comprises a hinge sequence with reduced effector function.

138. The method of embodiment 137, wherein the hinge sequence comprises or consists of the amino acid sequence of any one of SEQ ID NO:66, SEQ ID NO:67, SEQ ID NO:70 and SEQ ID NO:71.

139. The method of embodiment 137, wherein the hinge sequence comprises any hinge modification set forth in Section 6.2.6.2.

140. The method of any one of embodiments 126 to 139, wherein each constant domain comprises an amino acid sequence having at least 90% sequence identity to SEQ ID NO:46, wherein:
(a) both constant domains comprise the P-V-A-absent sequence at amino acid positions 233-236 (EU numbering);
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

141. The method of embodiment 140, wherein each constant domain comprises an amino acid sequence having at least 93% sequence identity to SEQ ID NO:46.

142. The method of embodiment 140, wherein each constant domain comprises an amino acid sequence having at least 95% sequence identity to SEQ ID NO:46.

143. The method of embodiment 140, wherein each constant domain comprises an amino acid sequence having at least 97% sequence identity to SEQ ID NO:46.

144. The method of any one of embodiments 126 to 139, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:58, provided that if the amino acid sequence has less than 100% identity to SEQ ID NO:58 the sequence retains the PVA modification (P-V-A-absent at amino acid positions 233-236 (EU numbering)) in the hinge and the knob mutation T366W; and
(b) a second constant domain comprising an amino acid sequence having at least 90%, at least 93%, at least 95%, at least 96%, at least 97%, at least 98%, at least 99% or 100% sequence identity to SEQ ID NO: 62, provided that if the amino acid sequence has less than 100% identity to SEQ ID NO:62 the sequence retains the PVA modification (P-V-A-absent at amino acid positions 233-236 (EU numbering)) in the hinge and the hole mutations T366S, L368A and Y407V.

145. The method of embodiment 144, wherein the first constant domain has at least 95% (or 100%) sequence identity to SEQ ID NO:58 and the second constant domain has at least 95% (or 100%) sequence identity to SEQ ID NO:62.

146. The method of any one of embodiments 126 to 139, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:58, provided that if the amino acid sequence has less than 100% identity to SEQ ID NO:58 the sequence retains the PVA modification (P-V-A-absent at amino acid positions 233-236 (EU numbering)) in the hinge and the knob mutation T366W; and
(b) a second constant domain comprising an amino acid sequence having at least 90%, at least 93%, at least 95%, at least 96%, at least 97%, at least 98%, at least 99% or 100% sequence identity to SEQ ID NO: 63, provided that if the amino acid sequence has less than 100% identity to SEQ ID NO:63 the sequence retains the PVA modification (P-V-A-absent at amino acid positions 233-236 (EU numbering)) in the hinge, the hole mutations T366S, L368A and Y407V, and the star mutations H435R and Y436F.

147. The method of embodiment 146, wherein the first constant domain has at least 95% (or 100%) sequence identity to SEQ ID NO:58 and the second constant domain has at least 95% (or 100%) sequence identity to SEQ ID NO:63.

148. The method of any one of embodiments 126 to 139, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:59, provided that if the amino acid sequence has less than 100% identity to SEQ ID NO:59 the sequence retains the PVA modification (P-V-A-absent at amino acid positions 233-236 (EU numbering)) in the hinge, the knob mutation T366W, and the star mutations H435R and Y436F; and
(b) a second constant domain comprising an amino acid sequence having at least 90%, at least 93%, at least 95%, at least 96%, at least 97%, at least 98%, at least 99% or 100% sequence identity to SEQ ID NO: 62, provided that if the amino acid sequence has less than 100% identity to SEQ ID NO:62 the sequence retains the PVA modification (P-V-A-absent at amino acid positions 233-236 (EU numbering)) in the hinge and the hole mutations T366S, L368A and Y407V.

149. The method of embodiment 148, wherein the first constant domain has at least 95% (or 100%) sequence identity to SEQ ID NO:59 and the second constant domain has at least 95% (or 100%) sequence identity to SEQ ID NO:62.

150. The method of any one of embodiments 126 to 139, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:59, provided that if the amino acid sequence has less than 100% identity to SEQ ID NO:59 the sequence retains the PVA modification (P-V-A-absent at amino acid positions 233-236 (EU numbering)) in the hinge, the knob mutation T366W, and the star mutations H435R and Y436F; and
(b) a second constant domain comprising an amino acid sequence having at least 90%, at least 93%, at least 95%, at least 96%, at least 97%, at least 98%, at least 99% or 100% sequence identity to SEQ ID NO: 63, provided that if the amino acid sequence has less than 100% identity to SEQ ID NO:63 the sequence retains the PVA modification (P-V-A-absent at amino acid positions 233-236 (EU numbering)) in the hinge, the hole mutations T366S, L368A and Y407V, and the star mutations H435R and Y436F.

151. The method of embodiment 150, wherein the first constant domain has at least 95% (or 100%) sequence identity to SEQ ID NO:59 and the second constant domain has at least 95% (or 100%) sequence identity to SEQ ID NO:62.

152. The method of any one of embodiments 126 to 139, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:60, provided that if the amino acid sequence has less than 100% identity to SEQ ID NO:60 the sequence retains the PVA modification (P-V-A-absent at amino acid positions 233-236 (EU numbering)) in the hinge, the disulfide architecture mutation S354C, and the knob mutation T366W; and
(b) a second constant domain comprising an amino acid sequence having at least 90%, at least 93%, at least 95%, at least 96%, at least 97%, at least 98%, at least 99% or 100% sequence identity to SEQ ID NO: 64, provided that if the amino acid sequence has less than 100% identity to SEQ ID NO:64 the sequence retains the PVA modification (P-V-A-absent at amino acid positions 233-236 (EU numbering)) in the hinge, the disulfide architecture mutation S354C, and the hole mutations T366S, L368A and Y407V.

153. The method of embodiment 152, wherein the first constant domain has at least 95% (or 100%) sequence identity to SEQ ID NO:60 and the second constant domain has at least 95% (or 100%) sequence identity to SEQ ID NO:64.

154. The method of any one of embodiments 126 to 139, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:60, provided that if the amino acid sequence has less than 100% identity to SEQ ID NO:60 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), and the knob mutation T366W; and
(b) a second constant domain comprising an amino acid sequence having at least 90%, at least 93%, at least 95%, at least 96%, at least 97%, at least 98%, at least 99% or 100% sequence identity to SEQ ID NO: 65, provided that if the amino acid sequence has less than 100% identity to SEQ ID NO:65 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the hole mutations T366S, L368A and Y407V, and the star mutations H435R and Y436F.

155. The method of embodiment 154, wherein the first constant domain has at least 95% (or 100%) sequence identity to SEQ ID NO:60 and the second constant domain has at least 95% (or 100%) sequence identity to SEQ ID NO:65.

156. The method of any one of embodiments 126 to 139, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:61, provided that if the amino acid sequence has less than 100% identity to SEQ ID NO:61 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the knob mutation T366W, and the star mutations H435R and Y436F; and
(b) a second constant domain comprising an amino acid sequence having at least 90%, at least 93%, at least 95%, at least 96%, at least 97%, at least 98%, at least 99% or 100% sequence identity to SEQ ID NO: 64, provided that if the amino acid sequence has less than 100% identity to SEQ ID NO:64 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), and the hole mutations T366S, L368A and Y407V.

157. The method of embodiment 156, wherein the first constant domain has at least 95% (or 100%) sequence identity to SEQ ID NO:61 and the second constant domain has at least 95% (or 100%) sequence identity to SEQ ID NO:64.

158. The method of any one of embodiments 126 to 139, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:61, provided that if the amino acid sequence has less than 100% identity to SEQ ID NO:61 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the knob mutation T366W, and the star mutations H435R and Y436F; and
(b) a second constant domain comprising an amino acid sequence having at least 90%, at least 93%, at least 95%, at least 96%, at least 97%, at least 98%, at least 99% or 100% sequence identity to SEQ ID NO: 65, provided that if the amino acid sequence has less than 100% identity to SEQ ID NO:65 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the hole mutations T366S, L368A and Y407V, and the star mutations H435R and Y436F.

159. The method of embodiment 158, wherein the first constant domain has at least 95% (or 100%) sequence identity to SEQ ID NO:61 and the second constant domain has at least 95% (or 100%) sequence identity to SEQ ID NO:65.

160. The method of any one of embodiments 126 to 139, wherein the constant domains each comprise an amino acid sequence having at least 90%, at least 93%, at least 95%, at least 96%, at least 97%, or at least 98% sequence identity to SEQ ID NO:49 (hIgG1 N180G, also referred to as hIgG1 N297G), wherein:
(a) both constant domains comprise the N180G/N297G amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

161. The method of embodiment 160, wherein each constant domain has at least 95% sequence identity to SEQ ID N0:49.

162. The method of any one of embodiments 126 to 139, wherein the constant domains each comprise an amino acid sequence having at least 90%, at least 93%, at least 95%, at least 96%, at least 97%, or at least 98% sequence identity to SEQ ID NO:53 (hIgG4 S108P, also referred to as hIgG4 S228P), wherein:
(a) both constant domains comprise the S108P/S228P amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

163. The method of embodiment 162, wherein each constant domain has at least 95% sequence identity to SEQ ID N0:49.

164. The method of any one of embodiments 126 to 139, wherein the constant domains each comprise an amino acid sequence having at least 90%, at least 93%, at least 95%, at least 96%, at least 97%, or at least 98% sequence identity to SEQ ID NO:54 (variant IgG4 with S108P, also referred to as hIgG4 S228P, substitution and IgG1 CH2 and CH3 domains), wherein:
(a) both constant domains comprise the S108P/S228P amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

165. The method of embodiment 164, wherein each constant domain has at least 95% sequence identity to SEQ ID N0:49.

166. The method of any one of embodiments 71 to 165, wherein the method is effective to reduce the weight of the subject.

167. The method of embodiment 71 to 166, wherein the method is effective to reduce circulating high-density lipoprotein cholesterol in the subject.

168. The method of embodiment 71 to 167, wherein the method is effective to increase circulating low-density lipoprotein cholesterol in the subject.

169. The method of embodiment 71 to 168, wherein the method is effective to reduce blood triglycerides in the subject.

170. The method of embodiment 71 to 169, wherein the method is effective to reduce blood glucose in the subject.

171. The method of embodiment 71 to 170, wherein the subject has a metabolic disorder.

172. The method of embodiment 171, wherein the metabolic disorder is metabolic syndrome.

173. The method of embodiment 171, wherein the metabolic disorder is obesity.

174. The method of embodiment 171, wherein the metabolic disorder is fatty liver.

175. The method of embodiment 171, wherein the metabolic disorder is hyperinsulinemia.

176. The method of embodiment 171, wherein the metabolic disorder is type 2 diabetes.

177. The method of embodiment 171, wherein the metabolic disorder is nonalcoholic steatohepatitis (“NASH”).

178. The method of embodiment 171, wherein the metabolic disorder is nonalcoholic fatty liver disease (“NAFLD”).

179. The method of embodiment 171, wherein the metabolic disorder is hypercholesterolemia.

180. The method of embodiment 171, wherein the metabolic disorder is hyperglycemia.

181. A multispecific binding molecule (MBM), comprising:
(a) an antigen-binding module 1 (ABM1) that specifically binds to human fibroblast growth factor receptor 1c isoform (“FGFR1c”);
(b) an antigen-binding module 2 (ABM2) that specifically binds to the GH1 domain of human klotho beta (“KLB”); and
(c) an antigen-binding module 3 (ABM3) that specifically binds to the GH2 domain of human KLB.

182. The MBM of embodiment 181, wherein each antigen-binding module is capable of binding its respective target at the same time as each of the other antigen-binding modules is bound to its respective target.

183. The MBM of embodiment 181 or embodiment 182, wherein ABM1 binds to loop D3 of FGFR1c.

184. The MBM of embodiment 181 or embodiment 182, wherein ABM1 binds to loop D2 of FGFR1c.

185. The MBM of any one of embodiments 181 to 184, which is a trispecific binding molecule (“TBM”).

186. The MBM of any of embodiments 181 to 185, wherein ABM1 is an antibody fragment, an scFv, a dsFv, a Fv, a Fab, an scFab, a (Fab′)2, a single domain antibody (SDAB), a VH or VL domain, or a camelid VHH domain.

187. The MBM of any of embodiments 181 to 186, wherein ABM2 is an antibody fragment, an scFv, a dsFv, a Fv, a Fab, an scFab, a (Fab′)2, a single domain antibody (SDAB), a VH or VL domain, or a camelid VHH domain.

188. The MBM of any of embodiments 181 to 187, wherein ABM3 is an antibody fragment, an scFv, a dsFv, a Fv, a Fab, an scFab, a (Fab′)2, a single domain antibody (SDAB), a VH or VL domain, or a camelid VHH domain.

189. The MBM of any one of embodiments 181 to 188, wherein ABM1 is an scFv.

190. The MBM of any one of embodiments 181 to 188, wherein ABM1 is a Fab.

191. The MBM of embodiment 190, in which a light chain of ABM1 is a universal light chain.

192. The MBM of embodiment 190, in which a light chain constant region and a first heavy chain constant region (CH1) of ABM1 are in a Crossmab arrangement.

193. The MBM of any one of embodiments 181 to 192, wherein ABM2 is an scFv.

194. The MBM of any one of embodiments 181 to 190, wherein ABM2 is a Fab.

195. The MBM of embodiment 194, in which a light chain of ABM2 is a universal light chain.

196. The MBM of embodiment 194, in which a light chain constant region and a first heavy chain constant region (CH1) of ABM2 are in a Crossmab arrangement.

197. The MBM of any one of embodiments 181 to 196, wherein ABM3 is an scFv.

198. The MBM of any one of embodiments 181 to 196, wherein ABM3 is a Fab.

199. The MBM of embodiment 198, in which a light chain of ABM3 is a universal light chain.

200. The MBM of embodiment 198, in which a light chain constant region and the first heavy chain constant region (CH1) of ABM3 are in a Crossmab arrangement.

201. The MBM of any one of embodiments 181 to 200, which comprises an Fc heterodimer.

202. The MBM of embodiment 201, wherein the Fc domains in the Fc heterodimer comprise knob-in-hole mutations as compared to a wild type Fc domain.

203. The MBM of embodiment 201, wherein the Fc domains in the Fc heterodimer comprise star mutations as compared to a wild type Fc domain.

204. The MBM of any one of embodiments 201 to 203, which comprises:
(a) a first polypeptide chain comprising, in an N- to C-terminal orientation, (i) an scFv operably linked to (ii) a first heavy chain region of a first Fab operably linked to (iii) an Fc domain;
(b) a second polypeptide chain comprising, in an N- to C-terminal orientation, (i) a second heavy chain region of a second Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.

205. The MBM of embodiment 204, in which the first light chain and the second light chain are identical.

206. The MBM of embodiment 204 or embodiment 205, in which ABM1 is the first Fab.

207. The MBM of embodiment 206, in which ABM2 is the scFv and ABM3 is the second Fab.

208. The MBM of embodiment 206, in which ABM2 is the second Fab and ABM3 is the scFv.

209. The MBM of any one of embodiments 204 to 208, in which the scFv is linked to the first heavy chain region via a linker.

210. The MBM of embodiment 209, wherein the linker is:
(a) at least 5 amino acids, at least 6 amino acids or at least 7 amino acids in length; and optionally

(b) up to 30 amino acids, up to 40 amino acids, up to 50 amino acids or up to 60 amino acids in length.

211. The MBM of embodiment 210, wherein the linker is:
(a) 5 amino acids to 50 amino acids in length;
(b) 5 amino acids to 45 amino acids in length;
(c) 5 amino acids to 40 amino acids in length;
(d) 5 amino acids to 35 amino acids in length;
(e) 5 amino acids to 30 amino acids in length;
(f) 5 amino acids to 25 amino acids in length; or
(g) 5 amino acids to 20 amino acids in length.

212. The MBM of embodiment 210, wherein the linker is:
(a) 6 amino acids to 50 amino acids in length;
(b) 6 amino acids to 45 amino acids in length;
(c) 6 amino acids to 40 amino acids in length;
(d) 6 amino acids to 35 amino acids in length;
(e) 6 amino acids to 30 amino acids in length;
(f) 6 amino acids to 25 amino acids in length; or
(g) 6 amino acids to 20 amino acids in length.

213. The MBM of embodiment 210, wherein the linker is:
(a) 7 amino acids to 40 amino acids in length;
(b) 7 amino acids to 35 amino acids in length;
(c) 7 amino acids to 30 amino acids in length;
(d) 7 amino acids to 25 amino acids in length;
(e) 7 amino acids to 20 amino acids in length.

214. The MBM of embodiment 210, wherein the linker is 5 amino acids to 45 amino acids in length.

215. The MBM of embodiment 210, wherein the linker is 7 amino acids to 30 amino acids in length.

216. The MBM of embodiment 210, wherein the linker is 5 amino acids to 25 amino acids in length.

217. The MBM of embodiment 210, wherein the linker is 10 amino acids to 60 amino acids in length.

218. The MBM of embodiment 217, wherein the linker is 20 amino acids to 50 amino acids in length.

219. The MBM of embodiment 218, wherein the linker is 25 amino acids to 35 amino acids in length.

220. The MBM of any one of embodiments 209 to 219, wherein the linker is or comprises a multimer of GₙS (SEQ ID NO: 15) or SGₙ (SEQ ID NO: 16), where n is an integer from 1 to 7.

221. The MBM of embodiment 220, wherein the linker is or comprises a multimer of G45 (SEQ ID NO: 17).

222. The MBM of any one of embodiments 204 to 219, wherein the linker is or comprises a comprises two consecutive glycines (2Gly), three consecutive glycines (3Gly), four consecutive glycines (4Gly (SEQ ID NO: 18)), five consecutive glycines (5Gly (SEQ ID NO: 19)), six consecutive glycines (6Gly (SEQ ID NO: 20)), seven consecutive glycines (7Gly (SEQ ID NO: 21)), eight consecutive glycines (8Gly (SEQ ID NO: 22)) or nine consecutive glycines (9Gly (SEQ ID NO: 23)).

223. The MBM of any one of embodiments 201 to 203, which comprises:
(a) a first polypeptide chain comprising, in an N- to C-terminal orientation, (i) a first heavy chain region of a first Fab operably linked to (ii) a second heavy chain region of a second Fab operably linked to (iii) an Fc domain, optionally wherein optionally the first heavy chain region is linked to the second heavy chain regions via a linker, optionally wherein the linker is as defined in any one of embodiments 32 to 44;
(b) a second polypeptide chain comprising, in an N- to C-terminal orientation, (i) a third heavy chain region of a third Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab; and
(e) a fifth polypeptide chain comprising a third light chain that pairs with the third heavy chain region to form the third Fab.

224. The MBM of embodiment 223, in which the first, second and third Fabs are the only antigen binding modules.

225. The MBM of any one of embodiments 223 to 224 in which the first light chain and the second light chain are identical.

226. The MBM of any one of embodiments 223 to 225, in which ABM1 is the second Fab.

227. The MBM of embodiment 226, in which ABM2 is the first Fab and ABM3 is the third Fab.

228. The MBM of embodiment 226, in which ABM3 is the first Fab and ABM2 is the third Fab.

229. The MBM of any one of embodiments 181 to 228, in which ABM1 comprises CDR sequences set forth in Table 1B.

230. The MBM of any one of embodiments 181 to 229, in which ABM2 comprises CDR sequences set forth in Table 2B.

231. The MBM of any one of embodiments 181 to 230, in which ABM3 comprises CDR sequences set forth in Table 3B.

232. The MBM of any one of embodiments 181 to 231, which is a trivalent MBM.

233. The MBM of any one of embodiments 181 to 231, which is a tetravalent MBM.

234. A multispecific binding molecule (MBM), comprising:
(a) a first antigen-binding means for specifically binding to human fibroblast growth factor receptor 1c isoform (“FGFR1c”);
(b) a second antigen-binding means for specifically binding to the GH1 domain of human klotho beta (“KLB”); and
(c) a third antigen-binding means for specifically binding to the GH2 domain of human KLB.

235. The MBM of embodiment 234, wherein each antigen-binding means is capable of binding its respective target at the same time as each of the other antigen-binding means is bound to its respective target.

236. The MBM of embodiment 234 or embodiment 235, wherein the first antigen-binding means binds to loop D3 of FGFR1c.

237. The MBM of embodiment 234 or embodiment 235, wherein the first antigen-binding means binds to loop D2 of FGFR1c.

238. The MBM of any one of embodiments 234 to 237, which is a trispecific binding molecule (“TBM”).

239. The MBM of any of embodiments 234 to 238, wherein the first antigen-binding means is an antibody fragment, an scFv, a dsFv, a Fv, a Fab, an scFab, a (Fab′)2, a single domain antibody (SDAB), a VH or VL domain, or a camelid VHH domain.

240. The MBM of any of embodiments 234 to 239, wherein the second antigen-binding means is an antibody fragment, an scFv, a dsFv, a Fv, a Fab, an scFab, a (Fab′)2, a single domain antibody (SDAB), a VH or VL domain, or a camelid VHH domain.

241. The MBM of any of embodiments 234 to 240, wherein the third antigen-binding means is an antibody fragment, an scFv, a dsFv, a Fv, a Fab, an scFab, a (Fab′)2, a single domain antibody (SDAB), a VH or VL domain, or a camelid VHH domain.

242. The MBM of any one of embodiments 234 to 241, wherein the first antigen-binding means is an scFv.

243. The MBM of any one of embodiments 234 to 241, wherein the first antigen-binding means is a Fab.

244. The MBM of embodiment 243, in which a light chain of the first antigen-binding means is a universal light chain.

245. The MBM of embodiment 243, in which a light chain constant region and a first heavy chain constant region (CH1) of the first antigen-binding means are in a Crossmab arrangement.

246. The MBM of any one of embodiments 234 to 245, wherein the second antigen-binding means is an scFv.

247. The MBM of any one of embodiments 234 to 243, wherein the second antigen-binding means is a Fab.

248. The MBM of embodiment 247, in which a light chain of the second antigen-binding means is a universal light chain.

249. The MBM of embodiment 247, in which a light chain constant region and the first heavy chain constant region (CH1) of the second antigen-binding means are in a Crossmab arrangement.

250. The MBM of any one of embodiments 234 to 249, wherein the third antigen-binding means is an scFv.

251. The MBM of any one of embodiments 234 to 249, wherein the third antigen-binding means is a Fab.

252. The MBM of embodiment 251, in which a light chain of the third antigen-binding means is a universal light chain.

253. The MBM of embodiment 251, in which a light chain constant region and the first heavy chain constant region (CH1) of the third antigen-binding means are in a Crossmab arrangement.

254. The MBM of any one of embodiments 234 to 253, which comprises an Fc heterodimer.

255. The MBM of embodiment 254, wherein the Fc domains in the Fc heterodimer comprise knob-in-hole mutations as compared to a wild type Fc domain.

256. The MBM of embodiment 254, wherein the Fc domains in the Fc heterodimer comprise star mutations as compared to a wild type Fc domain.

257. The MBM of any one of embodiments 254 to 256, which comprises:
(a) a first polypeptide chain comprising, in an N- to C-terminal orientation, (i) an scFv operably linked to (ii) a first heavy chain region of a first Fab operably linked to (iii) an Fc domain;
(b) a second polypeptide chain comprising, in an N- to C-terminal orientation, (i) a second heavy chain region of a second Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.

258. The MBM of embodiment 257, in which the first light chain and the second light chain are identical.

259. The MBM of embodiment 257 or embodiment 258, in which the first antigen-binding means is the first Fab.

260. The MBM of embodiment 259, in which the second antigen-binding means is the scFv and the third antigen-binding means is the second Fab.

261. The MBM of embodiment 259, in which the second antigen-binding means is the second Fab and the third antigen-binding means is the scFv.

262. The MBM of any one of embodiments 257 to 261, in which the scFv is linked to the first heavy chain region via a linker.

263. The MBM of embodiment 262, wherein the linker is:
(a) at least 5 amino acids, at least 6 amino acids or at least 7 amino acids in length; and optionally
(b) up to 30 amino acids, up to 40 amino acids, up to 50 amino acids or up to 60 amino acids in length.

264. The MBM of embodiment 263, wherein the linker is:
(a) 5 amino acids to 50 amino acids in length;
(b) 5 amino acids to 45 amino acids in length;
(c) 5 amino acids to 40 amino acids in length;
(d) 5 amino acids to 35 amino acids in length;
(e) 5 amino acids to 30 amino acids in length;
(f) 5 amino acids to 25 amino acids in length; or
(g) 5 amino acids to 20 amino acids in length.

265. The MBM of embodiment 263, wherein the linker is:
(a) 6 amino acids to 50 amino acids in length;
(b) 6 amino acids to 45 amino acids in length;
(c) 6 amino acids to 40 amino acids in length;
(d) 6 amino acids to 35 amino acids in length;
(e) 6 amino acids to 30 amino acids in length;
(f) 6 amino acids to 25 amino acids in length; or
(g) 6 amino acids to 20 amino acids in length.

266. The MBM of embodiment 263, wherein the linker is:
(a) 7 amino acids to 40 amino acids in length;
(b) 7 amino acids to 35 amino acids in length;
(c) 7 amino acids to 30 amino acids in length;
(d) 7 amino acids to 25 amino acids in length;
(e) 7 amino acids to 20 amino acids in length.

267. The MBM of embodiment 263, wherein the linker is 5 amino acids to 45 amino acids in length.

268. The MBM of embodiment 263, wherein the linker is 7 amino acids to 30 amino acids in length.

269. The MBM of embodiment 263, wherein the linker is 5 amino acids to 25 amino acids in length.

270. The MBM of embodiment 263, wherein the linker is 10 amino acids to 60 amino acids in length.

271. The MBM of embodiment 270, wherein the linker is 20 amino acids to 50 amino acids in length.

272. The MBM of embodiment 271, wherein the linker is 25 amino acids to 35 amino acids in length.

273. The MBM of any one of embodiments 257 to 272, wherein the linker is or comprises a multimer of GₙS (SEQ ID NO: 15) or SGₙ (SEQ ID NO: 16), where n is an integer from 1 to 7.

274. The MBM of embodiment 273, wherein the linker is or comprises a multimer of G45 (SEQ ID NO: 17).

275. The MBM of any one of embodiments 257 to 272, wherein the linker is or comprises a comprises two consecutive glycines (2Gly), three consecutive glycines (3Gly), four consecutive glycines (4Gly (SEQ ID NO: 18)), five consecutive glycines (5Gly (SEQ ID NO: 19)), six consecutive glycines (6Gly (SEQ ID NO: 20)), seven consecutive glycines (7Gly (SEQ ID NO: 21)), eight consecutive glycines (8Gly (SEQ ID NO: 22)) or nine consecutive glycines (9Gly (SEQ ID NO: 23)).

276. The MBM of any one of embodiments 254 to 256, which comprises:
(a) a first polypeptide chain comprising, in an N- to C-terminal orientation, (i) a first heavy chain region of a first Fab operably linked to (ii) a second heavy chain region of a second Fab operably linked to (iii) an Fc domain, optionally wherein optionally the first heavy chain region is linked to the second heavy chain regions via a linker, optionally wherein the linker is as defined in any one of embodiments 32 to 44;
(b) a second polypeptide chain comprising, in an N- to C-terminal orientation, (i) a third heavy chain region of a third Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab; and
(e) a fifth polypeptide chain comprising a third light chain that pairs with the third heavy chain region to form the third Fab.

277. The MBM of embodiment 276, in which the first, second and third Fabs are the only antigen binding modules.

278. The MBM of any one of embodiments 276 to 277 in which the first light chain and the second light chain are identical.

279. The MBM of any one of embodiments 276 to 278, in which the first antigen-binding means is the second Fab.

280. The MBM of embodiment 279, in which the second antigen-binding means is the first Fab and the third antigen-binding means is the third Fab.

281. The MBM of embodiment 279, in which the third antigen-binding means is the first Fab and the second antigen-binding means is the third Fab.

282. The MBM of any one of embodiments 234 to 281, in which the first antigen-binding means comprises CDR sequences set forth in Table 1B.

283. The MBM of any one of embodiments 234 to 282, in which the second antigen-binding means comprises CDR sequences set forth in Table 2B.

284. The MBM of any one of embodiments 234 to 283, in which the third antigen-binding means comprises CDR sequences set forth in Table 3B.

285. The MBM of any one of embodiments 234 to 284, which is a trivalent MBM.

286. The MBM of any one of embodiments 234 to 284, which is a tetravalent MBM.

287. The MBM of any one of embodiments 181 to 286, which comprises a heterodimeric pair of constant domains.

288. The MBM of embodiment 287, wherein each constant domain comprises one or more substitutions at S228, E233, L234, L235, D265, N297, P329 or P331 (all according to EU numbering).

289. The MBM of embodiment 288, wherein the constant domain comprises a S228P substitution.

290. The MBM of embodiment 288, wherein the constant domain comprises an E233A or E233P substitution.

291. The MBM of embodiment 288, wherein the constant domain comprises an L234A substitution.

292. The MBM of embodiment 288, wherein the constant domain comprises an L235A.

293. The MBM of embodiment 288, wherein the constant domain comprises a D265A substitution.

294. The MBM of embodiment 288, wherein the constant domain comprises an N297A or N297D substitution.

295. The MBM of embodiment 288, wherein the constant domain comprises a P329G or P329A substitution.

296. The MBM of embodiment 288, wherein the constant domain comprises a P331S.

297. The MBM of any one of embodiments 287 to 296, which comprises any combination of substitutions set forth in Section 6.2.7.1.

298. The MBM of any one of embodiments 287 to 297, wherein each constant domain comprises a hinge sequence with reduced effector function.

299. The MBM of embodiment 298, wherein the hinge sequence comprises or consists of the amino acid sequence of any one of SEQ ID NO:66, SEQ ID NO:67, SEQ ID NO:70 and SEQ ID NO:71.

300. The MBM of embodiment 298, wherein the hinge sequence comprises any hinge modification set forth in Section 6.2.6.2.

301. The MBM of any one of embodiments 287 to 300, wherein each constant domain comprises an amino acid sequence having at least 90% sequence identity to SEQ ID NO:46, wherein:
(a) both constant domains comprise the P-V-A-absent sequence at amino acid positions 233-236 (EU numbering);
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

302. The MBM of embodiment 301, wherein each constant domain comprises an amino acid sequence having at least 93% sequence identity to SEQ ID NO:46.

303. The MBM of embodiment 301, wherein each constant domain comprises an amino acid sequence having at least 95% sequence identity to SEQ ID NO:46.

304. The MBM of embodiment 301, wherein each constant domain comprises an amino acid sequence having at least 97% sequence identity to SEQ ID NO:46.

305. The MBM of any one of embodiments 287 to 300, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:58, provided that if the amino acid sequence has less than 100% identity to SEQ ID NO:58 the sequence retains the PVA modification (P-V-A-absent at amino acid positions 233-236 (EU numbering)) in the hinge and the knob mutation T366W; and
(b) a second constant domain comprising an amino acid sequence having at least 90%, at least 93%, at least 95%, at least 96%, at least 97%, at least 98%, at least 99% or 100% sequence identity to SEQ ID NO: 62, provided that if the amino acid sequence has less than 100% identity to SEQ ID NO:62 the sequence retains the PVA modification (P-V-A-absent at amino acid positions 233-236 (EU numbering)) in the hinge and the hole mutations T366S, L368A and Y407V.

306. The MBM of embodiment 305, wherein the first constant domain has at least 95% (or 100%) sequence identity to SEQ ID NO:58 and the second constant domain has at least 95% (or 100%) sequence identity to SEQ ID NO:62.

307. The MBM of any one of embodiments 287 to 300, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:58, provided that if the amino acid sequence has less than 100% identity to SEQ ID NO:58 the sequence retains the PVA modification (P-V-A-absent at amino acid positions 233-236 (EU numbering)) in the hinge and the knob mutation T366W; and
(b) a second constant domain comprising an amino acid sequence having at least 90%, at least 93%, at least 95%, at least 96%, at least 97%, at least 98%, at least 99% or 100% sequence identity to SEQ ID NO: 63, provided that if the amino acid sequence has less than 100% identity to SEQ ID NO:63 the sequence retains the PVA modification (P-V-A-absent at amino acid positions 233-236 (EU numbering)) in the hinge, the hole mutations T366S, L368A and Y407V, and the star mutations H435R and Y436F.

308. The MBM of embodiment 307, wherein the first constant domain has at least 95% (or 100%) sequence identity to SEQ ID NO:58 and the second constant domain has at least 95% (or 100%) sequence identity to SEQ ID NO:63.

309. The MBM of any one of embodiments 287 to 300, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:59, provided that if the amino acid sequence has less than 100% identity to SEQ ID NO:59 the sequence retains the PVA modification (P-V-A-absent at amino acid positions 233-236 (EU numbering)) in the hinge, the knob mutation T366W, and the star mutations H435R and Y436F; and
(b) a second constant domain comprising an amino acid sequence having at least 90%, at least 93%, at least 95%, at least 96%, at least 97%, at least 98%, at least 99% or 100% sequence identity to SEQ ID NO: 62, provided that if the amino acid sequence has less than 100% identity to SEQ ID NO:62 the sequence retains the PVA modification (P-V-A-absent at amino acid positions 233-236 (EU numbering)) in the hinge and the hole mutations T366S, L368A and Y407V.

310. The MBM of embodiment 309, wherein the first constant domain has at least 95% (or 100%) sequence identity to SEQ ID NO:59 and the second constant domain has at least 95% (or 100%) sequence identity to SEQ ID NO:62.

311. The MBM of any one of embodiments 287 to 300, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:59, provided that if the amino acid sequence has less than 100% identity to SEQ ID NO:59 the sequence retains the PVA modification (P-V-A-absent at amino acid positions 233-236 (EU numbering)) in the hinge, the knob mutation T366W, and the star mutations H435R and Y436F; and
(b) a second constant domain comprising an amino acid sequence having at least 90%, at least 93%, at least 95%, at least 96%, at least 97%, at least 98%, at least 99% or 100% sequence identity to SEQ ID NO: 63, provided that if the amino acid sequence has less than 100% identity to SEQ ID NO:63 the sequence retains the PVA modification (P-V-A-absent at amino acid positions 233-236 (EU numbering)) in the hinge, the hole mutations T366S, L368A and Y407V, and the star mutations H435R and Y436F.

312. The MBM of embodiment 311, wherein the first constant domain has at least 95% (or 100%) sequence identity to SEQ ID NO:59 and the second constant domain has at least 95% (or 100%) sequence identity to SEQ ID NO:62.

313. The MBM of any one of embodiments 287 to 300, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:60, provided that if the amino acid sequence has less than 100% identity to SEQ ID NO:60 the sequence retains the PVA modification (P-V-A-absent at amino acid positions 233-236 (EU numbering)) in the hinge, the disulfide architecture mutation S354C, and the knob mutation T366W; and
(b) a second constant domain comprising an amino acid sequence having at least 90%, at least 93%, at least 95%, at least 96%, at least 97%, at least 98%, at least 99% or 100% sequence identity to SEQ ID NO: 64, provided that if the amino acid sequence has less than 100% identity to SEQ ID NO:64 the sequence retains the PVA modification (P-V-A-absent at amino acid positions 233-236 (EU numbering)) in the hinge, the disulfide architecture mutation S354C, and the hole mutations T366S, L368A and Y407V.

314. The MBM of embodiment 313, wherein the first constant domain has at least 95% (or 100%) sequence identity to SEQ ID NO:60 and the second constant domain has at least 95% (or 100%) sequence identity to SEQ ID NO:64.

315. The MBM of any one of embodiments 287 to 300, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:60, provided that if the amino acid sequence has less than 100% identity to SEQ ID NO:60 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), and the knob mutation T366W; and
(b) a second constant domain comprising an amino acid sequence having at least 90%, at least 93%, at least 95%, at least 96%, at least 97%, at least 98%, at least 99% or 100% sequence identity to SEQ ID NO: 65, provided that if the amino acid sequence has less than 100% identity to SEQ ID NO:65 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the hole mutations T366S, L368A and Y407V, and the star mutations H435R and Y436F.

316. The MBM of embodiment 315, wherein the first constant domain has at least 95% (or 100%) sequence identity to SEQ ID NO:60 and the second constant domain has at least 95% (or 100%) sequence identity to SEQ ID NO:65.

317. The MBM of any one of embodiments 287 to 300, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:61, provided that if the amino acid sequence has less than 100% identity to SEQ ID NO:61 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the knob mutation T366W, and the star mutations H435R and Y436F; and
(b) a second constant domain comprising an amino acid sequence having at least 90%, at least 93%, at least 95%, at least 96%, at least 97%, at least 98%, at least 99% or 100% sequence identity to SEQ ID NO: 64, provided that if the amino acid sequence has less than 100% identity to SEQ ID NO:64 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), and the hole mutations T366S, L368A and Y407V.

318. The MBM of embodiment 317, wherein the first constant domain has at least 95% (or 100%) sequence identity to SEQ ID NO:61 and the second constant domain has at least 95% (or 100%) sequence identity to SEQ ID NO:64.

319. The MBM of any one of embodiments 287 to 300, wherein the constant domains comprise:
(a) a first constant domain comprising an amino acid sequence having at least 90%, at least 93%, at least 95%, at least 96%, at least 97%, at least 98%, at least 99% or 100% sequence identity to SEQ ID NO:61, provided that if the amino acid sequence has less than 100% identity to SEQ ID NO:61 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the knob mutation T366W, and the star mutations H435R and Y436F; and
(b) a second constant domain comprising an amino acid sequence having at least 90%, at least 93%, at least 95%, at least 96%, at least 97%, at least 98%, at least 99% or 100% sequence identity to SEQ ID NO: 65, provided that if the amino acid sequence has less than 100% identity to SEQ ID NO:65 the sequence retains the PVA modification (P-V-A-absent at amino acid positions 233-236 (EU numbering)) in the hinge, the disulfide architecture mutation S354C (or alternatively architecture mutation S354C is substituted with disulfide architecture mutation E356C), the hole mutations T366S, L368A and Y407V, and the star mutations H435R and Y436F.

320. The MBM of embodiment 319, wherein the first constant domain has at least 95% (or 100%) sequence identity to SEQ ID NO:61 and the second constant domain has at least 95% (or 100%) sequence identity to SEQ ID NO:65.

321. The MBM of any one of embodiments 287 to 300, wherein the constant domains each comprise an amino acid sequence having at least 90%, at least 93%, at least 95%, at least 96%, at least 97%, or at least 98% sequence identity to SEQ ID NO:49 (hIgG1 N180G, also referred to as hIgG1 N297G), wherein:
(a) both constant domains comprise the N180G/N297G amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

322. The MBM of embodiment 321, wherein each constant domain has at least 95% sequence identity to SEQ ID N0:49.

323. The MBM of any one of embodiments 287 to 300, wherein the constant domains each comprise an amino acid sequence having at least 90%, at least 93%, at least 95%, at least 96%, at least 97%, or at least 98% sequence identity to SEQ ID NO:53 (hIgG4 S108P, also referred to as hIgG4 S228P), wherein:
(a) both constant domains comprise the S108P/S228P amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

324. The MBM of embodiment 323, wherein each constant domain has at least 95% sequence identity to SEQ ID N0:49.

325. The MBM of any one of embodiments 287 to 300, wherein the constant domains each comprise an amino acid sequence having at least 90%, at least 93%, at least 95%, at least 96%, at least 97%, or at least 98% sequence identity to SEQ ID NO:54 (variant IgG4 with S108P, also referred to as hIgG4 S228P, substitution and IgG1 CH2 and CH3 domains), wherein:
(a) both constant domains comprise the S108P/S228P amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

326. The MBM of embodiment 325, wherein each constant domain has at least 95% sequence identity to SEQ ID N0:49.

327. A pharmaceutical composition comprising the MBM of any one of embodiments 181 to 326.

328. A method comprising administering the MBM of any one of embodiments 181 to 326, or the pharmaceutical composition of embodiment 327, to a subject.

329. The method of embodiment 328, wherein the MBM or the pharmaceutical composition is administered to the subject in an amount effective to:
(a) treat a metabolic condition; and/or
(b) improve metabolism.

330. The method of embodiment 328 or embodiment 329, wherein the method is effective to agonize FGF21 receptor complexes in the subject.

331. The method of any one of embodiments 328 to 330, wherein the subject has a metabolic disorder.

332. The method of embodiment 331, wherein the metabolic disorder is metabolic syndrome.

333. The method of embodiment 331, wherein the metabolic disorder is obesity.

334. The method of embodiment 331, wherein the metabolic disorder is fatty liver.

335. The method of embodiment 331, wherein the metabolic disorder is hyperinsulinemia.

336. The method of embodiment 331, wherein the metabolic disorder is type 2 diabetes.

337. The method of embodiment 331, wherein the metabolic disorder is nonalcoholic steatohepatitis (“NASH”).

338. The method of embodiment 331, wherein the metabolic disorder is hypercholesterolemia.

339. The method of embodiment 331, wherein the metabolic disorder is hyperglycemia.

340. A method of reducing weight, comprising administering to an overweight subject an effective amount of the MBM of any one of embodiments 181 to 326, or the pharmaceutical composition of embodiment 327.

341. The method of embodiment 340, wherein the subject is obese.

342. A method of treating nonalcoholic steatohepatitis (“NASH”), comprising administering to a subject suffering from NASH an effective amount of the MBM of any one of embodiments 181 to 286, or the pharmaceutical composition of embodiment 327.

343. A method of treating nonalcoholic fatty liver disease (NAFLD), comprising administering to a subject suffering from NAFLD an effective amount of the MBM of any one of embodiments 181 to 326, or the pharmaceutical composition of embodiment 327.

344. A method of reducing circulating HDL cholesterol, comprising administering to a subject suffering from elevated HDL levels an effective amount of the MBM of any one of embodiments 181 to 326, or the pharmaceutical composition of embodiment 327.

345. A method of increasing circulating LDL cholesterol, comprising administering to a subject suffering from low LDL levels an effective amount of the MBM of any one of embodiments 181 to 326, or the pharmaceutical composition of embodiment 327.

346. A method of reducing blood triglycerides, comprising administering to a subject suffering from elevated triglyceride levels an effective amount of the MBM of any one of embodiments 181 to 326, or the pharmaceutical composition of embodiment 327.

347. A method of reducing blood glucose, comprising administering to a subject suffering from elevated glucose levels effective amount of the MBM of any one of embodiments 181 to 326, or the pharmaceutical composition of embodiment 327.

348. A nucleic acid or plurality of nucleic acids encoding the MBM of any one of embodiments of any one of embodiments 181 to 326.

349. A cell engineered to express the MBM of any one of embodiments 181 to 326.

350. A cell transfected with one or more expression vectors comprising one or more nucleic acid sequences encoding the MBM of any one of embodiments 181 to 326 under the control of one or more promoters.

351. A method of producing a MBM, comprising:
(a) culturing the cell of embodiment 349 or 350 in conditions under which the MBM is expressed; and
(b) recovering the MBM from the cell culture 352. The method of embodiment 351, which further comprises enriching for the MBM.

353. The method of embodiment 351 or embodiment 352, which further comprises purifying the MBM.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. A method comprising administering a multispecific binding molecule (MBM) or a pharmaceutical composition comprising the MBM to a subject, wherein the MBM comprises:
(a) an antigen-binding module 1 (ABM1) that specifically binds to human fibroblast growth factor receptor 1c isoform (“FGFR1c”);
(b) an antigen-binding module 2 (ABM2) that specifically binds to the GH1 domain of human klotho beta (“KLB”); and
(c) an antigen-binding module 3 (ABM3) that specifically binds to the GH2 domain of human KLB.

2. The method of claim 1, wherein the MBM is a trispecific binding molecule (“TBM”).

3-5. (canceled)

6. The method of claim 1 wherein the MBM comprises an Fc heterodimer.

7. The method of claim 6, wherein the MBM comprises:
(a) a first polypeptide chain comprising, in an N- to C-terminal orientation, (i) an scFv operably linked to (ii) a first heavy chain region of a first Fab operably linked to (iii) an Fc domain;
(b) a second polypeptide chain comprising, in an N- to C-terminal orientation, (i) a second heavy chain region of a second Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.

8-10. (canceled)

11. The method of claim 7, in which the scFv is linked to the first heavy chain region via a linker.

12. The method of claim 11, wherein the linker is 5 amino acids to 45 amino acids in length.

13. (canceled)

14. The method of claim 6, wherein the MBM comprises:
(a) a first polypeptide chain comprising, in an N- to C-terminal orientation, (i) a first heavy chain region of a first Fab operably linked to (ii) a second heavy chain region of a second Fab operably linked to (iii) an Fc domain;
(b) a second polypeptide chain comprising, in an N- to C-terminal orientation, (i) a third heavy chain region of a third Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab; and
(e) a fifth polypeptide chain comprising a third light chain that pairs with the third heavy chain region to form the third Fab.

15-16. (canceled)

17. The method of claim 14, in which the first heavy chain region is linked to the second heavy chain region via a linker.

18. The method of claim 15, wherein the linker is 5 amino acids to 45 amino acids in length.

19. The method of claim 1, wherein the subject has a metabolic disorder.

20. (canceled)

21. The method of claim 1, wherein the MBM comprises a heterodimeric pair of constant domains.

22. The method of claim 21, wherein each constant domain comprises a hinge sequence with reduced effector function.

23. (canceled)

24. The method of claim 22, wherein each constant domain comprises an amino acid sequence having at least 90% sequence identity to SEQ ID NO:46, wherein:
(a) both constant domains comprise the P-V-A-absent sequence at amino acid positions 233-236 (EU numbering);
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

25. The method of claim 22, wherein the constant domains each comprise an amino acid sequence having at least 90% sequence identity to SEQ ID NO:49 (hIgG1 N180G, also referred to as hIgG1 N297G), wherein:
(a) both constant domains comprise the N180G/N297G amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

26. The method of claim 22, wherein the constant domains each comprise an amino acid sequence having at least 90% sequence identity to SEQ ID NO:53 (hIgG4 S108P, also referred to as hIgG4 S228P), wherein:
(a) both constant domains comprise the S108P/S228P amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

27. The method of claim 22, wherein the constant domains each comprise an amino acid sequence having at least 90% sequence identity to SEQ ID NO:54 (variant IgG4 with S108P, also referred to as hIgG4 S228P, substitution and IgG1 CH2 and CH3 domains), wherein:
(a) both constant domains comprise the S108P/S228P amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

28. A multispecific binding molecule (MBM), comprising:
(a) an antigen-binding module 1 (ABM1) that specifically binds to human fibroblast growth factor receptor 1c isoform (“FGFR1c”);
(b) an antigen-binding module 2 (ABM2) that specifically binds to the GH1 domain of human klotho beta (“KLB”); and
(c) an antigen-binding module 3 (ABM3) that specifically binds to the GH2 domain of human KLB.

29. The MBM of claim 28, which is a trispecific binding molecule (“TBM”).

30-32. (canceled)

33. The MBM of claim 28, which comprises an Fc heterodimer.

34. The MBM of claim 33, which comprises:
(a) a first polypeptide chain comprising, in an N- to C-terminal orientation, (i) an scFv operably linked to (ii) a first heavy chain region of a first Fab operably linked to (iii) an Fc domain;
(b) a second polypeptide chain comprising, in an N- to C-terminal orientation, (i) a second heavy chain region of a second Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab.

35-37. (canceled)

38. The MBM of claim 34, in which the scFv is linked to the first heavy chain region via a linker.

39. The MBM of claim 38, wherein the linker is 5 amino acids to 45 amino acids in length.

40. (canceled)

41. The MBM of claim 33, which comprises:
(a) a first polypeptide chain comprising, in an N- to C-terminal orientation, (i) a first heavy chain region of a first Fab operably linked to (ii) a second heavy chain region of a second Fab operably linked to (iii) an Fc domain;
(b) a second polypeptide chain comprising, in an N- to C-terminal orientation, (i) a third heavy chain region of a third Fab operably linked to (ii) an Fc domain;
(c) a third polypeptide chain comprising a first light chain that pairs with the first heavy chain region to form the first Fab;
(d) a fourth polypeptide chain comprising a second light chain that pairs with the second heavy chain region to form the second Fab; and
(e) a fifth polypeptide chain comprising a third light chain that pairs with the third heavy chain region to form the third Fab.

42-46. (canceled)

47. The MBM of claim 28, which comprises a heterodimeric pair of constant domains.

48. The MBM of claim 47, wherein each constant domain comprises a hinge sequence with reduced effector function.

49. (canceled)

50. The MBM of claim 47, wherein each constant domain comprises an amino acid sequence having at least 90% sequence identity to SEQ ID NO:46, wherein:
(a) both constant domains comprise the P-V-A-absent sequence at amino acid positions 233-236 (EU numbering);
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

51. The MBM of claim 47, wherein the constant domains each comprise an amino acid sequence having at least 90% sequence identity to SEQ ID NO:49 (hIgG1 N180G, also referred to as hIgG1 N297G), wherein:
(a) both constant domains comprise the N180G/N297G amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

52. The MBM of claim 47, wherein the constant domains each comprise an amino acid sequence having at least 90% sequence identity to SEQ ID NO:53 (hIgG4 S108P, also referred to as hIgG4 S228P), wherein:
(a) both constant domains comprise the S108P/S228P amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

53. The MBM of claim 47, wherein the constant domains each comprise an amino acid sequence having at least 90% sequence identity to SEQ ID NO:54 (variant IgG4 with S108P, also referred to as hIgG4 S228P, substitution and IgG1 CH2 and CH3 domains), wherein:
(a) both constant domains comprise the S108P/S228P amino acid substitution;
(b) one constant domain comprises knob mutation T366W and the other constant domain comprises hole mutations T366S, L368A and Y407V;
(c) optionally, one or both constant domains comprise star mutations H435R and Y436F; and
(d) both or neither constant domains comprise disulfide architecture mutation S354C or E356C.

54. A pharmaceutical composition comprising the MBM of claim 28.

55. A method comprising administering the MBM of claim 28 to a subject.

56. The method of claim 55, wherein the MBM is administered to the subject in an amount effective to:
(a) treat a metabolic condition; and/or
(b) improve metabolism.

57. The method of claim 55, wherein the subject has a metabolic disorder.

58. A nucleic acid or plurality of nucleic acids encoding the MBM of claim 28.

59. A cell engineered to express the MBM of claim 28.

60. (canceled)

61. A method of producing a MBM, comprising:
(a) culturing the cell of claim 59 in conditions under which the MBM is expressed; and
(b) recovering the MBM from the cell culture

62-63. (canceled)
